SEQUENCE LISTING (6 sequences)

SEQ ID NO: 1 - DNA, 20 bp, Unknown
  source 1..20: /mol_type=“unassigned DNA” /note=“L32 forward primer” /organism=“Unknown”
aagttcatccggcaccagtc

SEQ ID NO: 2 - DNA, 21 bp, Unknown
  source 1..21: /mol_type=“unassigned DNA” /note=“L32 revers primer” /organism=“Unknown”
tggcccttgaatcttctacga

SEQ ID NO: 3 - DNA, 22 bp, Unknown
  source 1..22: /mol_type=“unassigned DNA” /note=“L32 probe” /organism=“Unknown”
cccagaggcattgacaacaggg

SEQ ID NO: 4 - DNA, 20 bp, Unknown
  source 1..20: /mol_type=“unassigned DNA” /note=“AXIN2 forward primer” /organism=“Unknown”
aggccagtgagttggttgtc

SEQ ID NO: 5 - DNA, 20 bp, Unknown
  source 1..20: /mol_type=“unassigned DNA” /note=“AXIN2 revers primer” /organism=“Unknown”
agctctgagccttcagcatc

SEQ ID NO: 6 - DNA, 25 bp, Unknown
  source 1..25: /mol_type=“unassigned DNA” /note=“AXIN2 probe” /organism=“Unknown”
tctgtggggaagaaattccataccg

(19) United States
(12) Patent Application Publication
(10) Pub. No.: US 2018/0044306 A1
(43) Pub. Date: Feb. 15, 2018

(54) 1,3,4-THIADIAZOL-2-YL-BENZAMIDE DERIVATIVES AS INHIBITORS OF THE WNT SIGNALLING PATHWAY

(71) Applicant: Bayer Pharma Aktiengesellschaft, Berlin (DE)

(72) Inventors: Kai THEDE, Berlin (DE); Eckhard BENDER, Langenfeld (DE); William J. SCOTT, Guilford, CT (US); Anja GIESE, Berlin (DE); Ludwig ZORN, Berlin (DE); Ningshu LIU, Berlin (DE); Ursula MÖNNING, Woltersdorf (DE); Franziska SIEGEL, Berlin (DE); Stefan GOLZ, Mülheim an der Ruhr (DE); Andrea HÄGEBARTH, Berlin (DE); Philip LIENAU, Berlin (DE); Florian PÜHLER, Cambridge, MA (US); Daniel BASTING, Köln (DE); Dirk SCHNEIDER, Wuppertal (DE); Manfred MÖWES, Berlin (DE); Jens GEISLER, Berlin (DE)

(73) Assignee: Bayer Pharma Aktiengesellschaft, Berlin (DE)

(21) Appl. No.: 15/552,249

(22) PCT Filed: Feb. 16, 2016
(86) PCT No.: PCT/EP2016/053231

§ 371 Date: Aug. 18, 2017

(30) Foreign Application Priority Data
Feb. 20, 2015 (EP) 15155886.3

(51) Int. Cl. C07D 285/135; C07D 417/04; A61K 31/5377; A61K 45/06; A61K 31/496

(52) U.S. Cl. CPC C07D 285/135; A61K 45/06; A61K 31/496; A61K 31/5377; C07D 417/04

(57) ABSTRACT

The present invention relates to inhibitors of the Wnt signalling pathways of general formula (I) as described and defined herein, to methods of preparing said compounds, to intermediate compounds useful for preparing said compounds, to pharmaceutical compositions and combinations comprising said compounds and to the use of said compounds for manufacturing a pharmaceutical composition for the treatment or prophylaxis of a disease, in particular of a hyper-proliferative disorder, as a sole agent or in combination with other active ingredients.

The present invention relates to inhibitors of the Wnt signalling pathways of general formula (I) as described and defined herein, to methods of preparing said compounds, to intermediate compounds useful for preparing said compounds, to pharmaceutical compositions and combinations comprising said compounds and to the use of said compounds for manufacturing a pharmaceutical composition for the treatment or prophylaxis of a disease, in particular of a hyper-proliferative disorder, as a sole agent or in combination with other active ingredients.

### BACKGROUND

The Wnt signaling pathways are a group of signal transduction pathways made of proteins that pass signals from outside of a cell through cell surface receptors to the inside of the cell.

Wnt proteins are secreted glycoproteins with a molecular weight in the range of 39-46 kD, whereby in total 19 different members of the Wnt protein family are known (McMahon et al., Trends Genet. 8, 1992, 236-242). They are the ligands of so-called Frizzled receptors, which form a family of seven-transmembrane spanning receptors comprising 10 distinct subtypes. A certain Wnt ligand can thereby activate several different Frizzled receptor subtypes and vice versa a particular Frizzled receptor can be activated by different Wnt protein subtypes (Huang et al., Genome Biol. 5, 2004, 234.1-234.8).

Binding of a Wnt to its receptor can activate two different signaling cascades, one is called the non-canonical pathway, which involves CamK II and PKC (Kuhl et al., Trends Genet. 16 (7), 2000, 279-283). The other, the so-called canonical pathway (Tamai et al., Mol. Cell 13, 2004, 149-156) regulates the concentration of the transcription factor β-catenin.

In the case of non-stimulated canonical Wnt signaling, β-catenin is captured by a destruction complex consisting of adenomatous polyposis coli (APC), glycogen synthase kinase 3-β (GSK-3β), Axin-1 or -2 and Casein Kinase 1α. Captured β-catenin is then phosphorylated, ubiquitinated and subsequently degraded by the proteasome.

However, when a canonical Wnt activates the membrane complex of a Frizzled receptor and its Lipoprotein 5 or 6 (LRP 5/6) co-receptor, this leads to the recruitment of dishevelled (Dvl) by the receptors and subsequent phosphorylation of LRP 5/6, followed by binding of Axin-1 or Axin-2 to the membrane complex as well. The deprivation of Axin from the β-catenin destruction complex leads to the disassembly of the latter and β-catenin can reach the nucleus, where it together with TCF and LEF transcription factors and other transcriptional coregulators like Pygopus, BCL9/Legless, CDK8 module of Mediator and TRRAP initiates transcription of genes with promoters containing TCF elements (Najdi, J. Carcinogenesis 2011; 10:5).

The Wnt signaling cascade can be constitutively activated by mutations in genes involved in this pathway. This is especially well documented for mutations of the APC and axin genes, and also for mutations of the β-catenin phosphorylation sites, all of which are important for the development of colorectal and hepatocellular carcinomas (Polakis, EMBO J., 31, 2012, 2737-2746).

The Wnt signaling cascade has important physiological roles in embryonal development and tissue homeostasis the latter especially for hair follicles, bones and the gastrointestinal tract. Deregulation of the Wnt pathway can activate in a cell and tissue specific manner a number of genes known to be important in carcinogenesis. Among them are c-myc, cyclin D1, Axin-2 and metalloproteases (He et al., Science 281, 1998, 1509-1512).

Deregulated Wnt activity can drive cancer formation, increased Wnt signaling can thereby be caused through autocrine Wnt signaling, as shown for different breast, ovarian, prostate and lung carcinomas as well as for various cancer cell lines (Bafico, Cancer Cell 6, 2004, 497-506; Yee, Mol. Cancer 9, 2010, 162-176; Nguyen, Cell 138, 2009, 51-62).

For cancer stem cells (CSCs) it was shown that they have increased Wnt signaling activity and that its inhibition can reduce the formation of metastases (Vermeulen et al., Nature Cell Biol. 12 (5), 2010, 468-476; Polakis, EMBO J. 31, 2012, 2737-2746; Reya, Nature, 434, 2005, 843-850).

Furthermore, there is a lot of evidence supporting an important role of Wnt signaling in cardiovascular diseases. One aspect thereby is heart failure and cardiac hypertrophy where deletion of Dapper-1, an activator of the canonical β-catenin Wnt pathway has been shown to reduce functional impairement and hypertrophy (Hagenmueller, M. et al.: Dapper-1 induces myocardial remodeling through activation of canonical wnt signaling in cardiomyocytes; Hypertension, 61 (6), 2013, 1177-1183).

Additional support for a role of Wnt signaling in heart failure comes from animal experimental models and clinical studies with patients, in which it was shown, that the level of secreted frizzled related protein 3 (sFRP3) is associated with the progression of heart failure (Askevold, E. T. et al.: The cardiokine secreted Frizzled-related protein 3, a modulator of Wnt signaling in clinical and experimental heart failure; J. Intern Med., 2014 (doi:10.1111/joim.12175)). For cardiac remodeling and infarct healing the expression of Fzd2 receptors on myofibroblasts migrating into the infarct area has been demonstrated (Blankesteijn, W. M. et al.: A homologue of Drosophila tissue polarity gene frizzled is expressed in migrating myofibroblasts in the infarcted rat heart; Nat. Med. 3, 1997, 541-544). The manifold effects of Wnt signaling in heart failure, fibrosis and arrhythmias have been recently reviewed by Dawson et al. (Dawson, K. et al.: Role of the Wnt-Frizzled system in cardiac pathophysiology: a rapidly developing, poorly understood area with enormous potential; J. Physiol. 591 (6), 2013, 1409-1432).

For the vasculature, effects of Wnt signaling could be shown as well, mainly in respect to restenosis via enhancement of vascular smooth muscle cell proliferation (Tsaousi, A. et al.: Wnt4/b-catenin signaling induces VSMC proliferation and is associated with initmal thickening; Circ. Res. 108, 2011, 427-436).

Besides the effects on heart and vasculature, dysregulated Wnt signaling is also an important component in chronic kidney disease as could be shown for upregulated Wnt activity in immune cells from corresponding patients (Al-Chaqmaqchi, H. A. et al.: Activation of Wnt/b-catenin pathway in monocytes derived from chronic kidney disease patients; PLoS One, 8 (7), 2013, doi: 10.1371) and altered levels of secreted Wnt inhibitor in patient sera (de Oliveira, R. B. et al.: Disturbances of Wnt/b-catenin pathway and energy metabolism in early CKD: effect of phosphate binders; Nephrol. Dial. Transplant. (2013) 28 (10): 2510-2517).

In adults, mis-regulation of the Wnt pathway also leads to a variety of abnormalities and degenerative diseases. An LRP mutation has been identified that causes increased bone density at defined locations such as the jaw and palate (Boyden L M et al.: High bone density due to a mutation in LDL-receptor-related protein 5; N Engl J Med. 2002 May 16; 346(20):1513-21, Gong Y, et al.: LDL receptor-related protein 5 (LRPS) affects bone accrual and eye development; Cell 2001; 107:513-23). The mutation is a single amino-acid substitution that makes LRPS insensitive to Dkk-mediated Wnt pathway inhibition, indicating that the phenotype results from overactive Wnt signaling in the bone. Recent reports have suggested that Wnt signaling is an important regulator for adipogenesis or insulin secretion and might be involved in the pathogenesis of type 2 diabetes. It has been shown that expression of the Wnt5B gene was detectable in several tissues, including adipose, pancreas, and liver. Subsequent in vitro experiments identified the fact that expression of the Wnt5b gene was increased at an early phase of adipocyte differentiation in mouse 3T3-L1 cells. Furthermore, overexpression of the Wnt5b gene in preadipocytes resulted in the promotion of adipogenesis and the enhancement of adipocytokine-gene expression. These results indicate that the Wnt5B gene may contribute to conferring susceptibility to type 2 diabetes and may be involved in the pathogenesis of this disease through the regulation of adipocyte function (Kanazawa A, et al.: Association of the gene encoding wingless-type mammary tumor virus integration-site family member 58 (Wnt5B) with type 2 diabetes; Am J Hum Genet. 2004 November; 75(5):832-43)

Accordingly, identification of methods and compounds that modulate the Wnt—dependent cellular responses may offer an avenue for regulating physiological functions and therapeutic treatment of diseases associated with aberrant activity of the pathways.

Inhibitors of the Wnt signalling pathways are disclosed e.g. in US2008-0075714(A1), US2011-0189097(A1), US2012-0322717(A9), WO2010/014948(A1), WO2012/088712(A1), WO2012/140274(A2,A3) and WO2013/093508(A2).

WO 2005/084368(A2) discloses heteroalkyl-substituted biphenyl-4-carboxylic acid arylamide analogues and the use of such compounds for treating conditions related to capsaicin receptor activation, for identifying other agents that bind to capsaicin receptor, and as probes for the detection and localization of capsaicin receptors. The structural scope of the compounds claimed in claim 1 is huge, whereas the structural space spanned by the few examples is much smaller. There is no specific example which is covered by the formula (I) as described and defined herein.

WO 2000/55120(A1) and WO 2000/07991 (A1) disclose amide derivatives and their use for the treatment of cytokine mediated diseases. The few specific examples disclosed in WO 2000/55120(A1) and WO 2000/07991 (A1) are not covered by the formula (I) as described and defined herein.

WO 1998/28282 (A2) discloses oxygen or sulfur containing heteroaromatics as factor Xa inhibitors. The specific examples disclosed in WO 1998/28282 (A2) are not covered by the formula (I) as described and defined herein.

WO 2011/035321 (A1) discloses methods of treating Wnt/Frizzled-related diseases, comprising administering niclosamide compounds. According to the specification of WO 2011/035321 (A1) libraries of FDA-approved drugs were examined for their utility as Frizzled internalization modulators, employing a primary imaged-based GFP-fluorescence assay that used Frizzled 1 endocytosis as the readout. It was discovered that the antihelminthic niclosamide, a drug used for the treatment of tapeworms, promotes Frizzled 1 internalization (endocytosis), down regulates Dishevelled-2 protein, and inhibits Wnt3A-stimulated β-catenin stabilization and LEF/TCF reporter activity. The specific examples disclosed in WO 2011/035321 (A1) are not covered by the formula (I) as described and defined herein. Additionally, WO 2011/035321 (A1) does neither teach nor suggest the compounds of formula (I) as described and defined herein. The same is true for the related publication WO 2004/006906 (A2) which discloses a method for treating a patient having a cancer or other neoplasm by administering to the patient a niclosamide.

JP 2010-138079 (A) relates to amide derivatives exhibiting insecticidal effects. The specific examples disclosed in JP 2010-138079 (A) are not covered by the formula (I) as described and defined herein.

WO 2004/022536 (A1) relates to heterocyclic compounds that inhibit phosphodiesterase type 4 (PDE 4) and their use for treating inflammatory conditions, diseases of the central nervous system and insulin resistant diabetes. The specific examples disclosed in WO 2004/022536 (A1) are not covered by the formula (I) as described and defined herein.

### SUMMARY

The present invention relates to compounds of general formula (I):

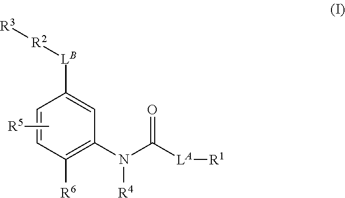

in which :
L^{A} represents
   *CH₂**;
   wherein * indicates the point of attachment to the carbonyl group, and ** indicates the point of attachment to R¹;
L^{B} represents *N(H)−C(═O)**;
   wherein * indicates the point of attachment to R², and ** indicates the point of attachment to the phenyl group;
R¹ represents a group selected from:

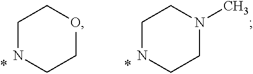

wherein * indicates the point of attachment to L^{A},
R² represents

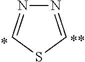

wherein * indicates the point of attachment to R³, and ** indicates the point of attachment to L^{B};
R³ represents a group selected from:

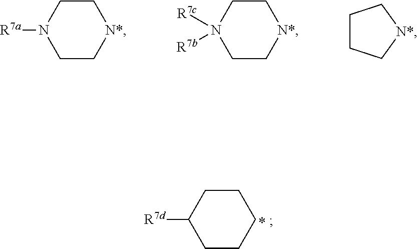

wherein * indicates the point of attachment to R²;
R⁴ represents a hydrogen atom;
R⁵ represents a hydrogen atom;
R⁶ represents a —O—CF₃ group;
R^{7a} represents a group selected from:
   —C(═O)—R⁸, —C(═O)—O—R⁸, —C(═O)—N(R⁸)(R⁹), —S(═O)₂—N(R⁸)(R⁹),
R^{7b} represents a hydrogen atom or a methyl-group;
R^{7c}represents a hydrogen atom or a group selected from:
   methyl-, —OH, HO—(C₁—C₃-alkyl)-, methoxy-, —C(═O)—O—R⁸;
R^{7d} represents a hydrogen atom;
R⁸ represents a group selected from:
   —CH₃, —CH₂-CH₃, —C(H)(CH₃)₂, —C(CH₃)₃, -cyclopropyl;
R⁹ represents a —CH₃ group;

or a tautomer, an N-oxide, a hydrate, a solvate, or a salt thereof, or a mixture of same.

The present invention further relates to a pharmaceutical composition comprising a compound of formula (I), supra.

The present invention further relates to the use of a compound of formula (I), supra, for the prophylaxis or treatment of a disease.

The present invention further relates to the use of a compound of formula (I), supra, for the preparation of a medicament for the prophylaxis or treatment of a disease.

The present invention further relates to methods of preparing a compound of formula (I), supra.

The present invention further relates to intermediate compounds useful for preparing a compound of formula (I), supra.

### DETAILED DESCRIPTION

The terms as mentioned in the present text have preferably the following meanings :

The term “halogen atom” or “halo-” is to be understood as meaning a fluorine, chlorine, bromine or iodine atom.

The term “C₁-C₆-alkyl” is to be understood as preferably meaning a linear or branched, saturated, monovalent hydrocarbon group having 1, 2, 3, 4, 5 or 6 carbon atoms, e.g. a methyl, ethyl, propyl, butyl, pentyl, hexyl, iso-propyl, iso-butyl, sec-butyl, tert-butyl, iso-pentyl, 2-methylbutyl, 1-methyl butyl, 1-ethyl propyl, 1,2-dimethylpropyl, neo-pentyl, 1,1-dimethylpropyl, 4-methyl pentyl, 3-methyl pentyl, 2-methylpentyl, 1-methylpentyl, 2-ethyl butyl, 1-ethyl butyl, 3,3-dimethyl butyl, 2,2-dimethylbutyl, 1,1-dimethylbutyl, 2,3-dimethylbutyl, 1,3-dimethylbutyl, or 1,2-dimethylbutyl group, or an isomer thereof. Particularly, said group has 1, 2, 3 or 4 carbon atoms (“C₁-C₄-alkyl”), e.g. a methyl, ethyl, propyl, butyl, iso-propyl, iso-butyl, sec-butyl, tert-butyl group, more particularly 1, 2 or 3 carbon atoms (“C₁-C₃-alkyl”), e.g. a methyl, ethyl, n-propyl- or iso-propyl group.

The term “halo-C₁-C₆-alkyl” is to be understood as preferably meaning a linear or branched, saturated, monovalent hydrocarbon group in which the term “C₁-C₆-alkyl” is defined supra, and in which one or more of the hydrogen atoms is replaced, identically or differently, by a halogen atom.

Particularly, said halogen atom is F. Said halo-C₁-C₆-alkyl group is, for example, —CF₃, —CHF₂, —CH₂F, —CF₂CF₃, or —CH₂CF₃.

The term “C₁-C₆-alkoxy” is to be understood as preferably meaning a linear or branched, saturated, monovalent group of formula —O—(C₁-C₆-alkyl), in which the term “C₁-C₆-alkyl” is defined supra, e.g. a methoxy, ethoxy, n-propoxy, iso-propoxy, n-butoxy, iso-butoxy, tert-butoxy, sec-butoxy, pentoxy, iso-pentoxy, or n-hexoxy group, or an isomer thereof.

The term “halo-C₁-C₆-alkoxy” is to be understood as preferably meaning a linear or branched, saturated, monovalent C₁-C₆-alkoxy group, as defined supra, in which one or more of the hydrogen atoms is replaced, identically or differently, by a halogen atom. Particularly, said halogen atom is F. Said halo-C₁-C₆-alkoxy group is, for example, —OCF₃, —OCHF₂, —OCH₂F, —OCF₂CF₃, or —OCH₂CF₃.

The term “C₁-C₆-alkoxy-C₁-C₆-alkyl” is to be understood as preferably meaning a linear or branched, saturated, monovalent C₁-C₆-alkyl group, as defined supra, in which one or more of the hydrogen atoms is replaced, identically or differently, by a C₁-C₆-alkoxy group, as defined supra, e.g. methoxyalkyl, ethoxyalkyl, propyloxyalkyl, iso-propoxyalkyl, butoxyalkyl, iso-butoxyalkyl, tert-butoxyalkyl, sec-butoxyalkyl, pentyloxyalkyl, iso-pentyloxyalkyl, hexyloxyalkyl group, or an isomer thereof.

The term “halo-C₁-C₆-alkoxy-C₁-C₆-alkyl” is to be understood as preferably meaning a linear or branched, saturated, monovalent C₁-C₆-alkoxy-C₁-C₆-alkyl group, as defined supra, in which one or more of the hydrogen atoms is replaced, identically or differently, by a halogen atom. Particularly, said halogen atom is F. Said halo-C₁-C₆-alkoxy-C₁-C₆-alkyl group is, for example, —CH₂CH₂OCF₃, —CH₂CH₂OCHF₂, —CH₂CH₂OCH₂F, —CH₂CH₂OCF₂CF₃, or -CH₂CH₂OCH₂CF₃.

The term “C1-C6-alkoxy-C2-C6-alkoxy” is to be understood as preferably meaning a saturated, monovalent C₂-C₆-alkoxy group, as defined supra, in which one of the hydrogen atoms is replaced by a C₁-C₆-alkoxy group, as defined supra, e.g. methoxyalkoxy, ethoxyalkoxy, pentoxyalkoxy, hexoxyalkoxy group or methoxyethoxy, ethoxyethoxy, iso-propoxyhexoxy group, in which the term “alkoxy” is defined supra, or an isomer thereof.

The term “C₂-C₆-alkenyl” is to be understood as preferably meaning a linear or branched, monovalent hydrocarbon group, which contains one or more double bonds, and which has 2, 3, 4, 5 or 6 carbon atoms, particularly 2 or 3 carbon atoms (“C₂-C₃-alkenyl”), it being understood that in the case in which said alkenyl group contains more than one double bond, then said double bonds may be isolated from, or conjugated with, each other. Said alkenyl group is, for example, a vinyl, allyl, (E)-2-methylvinyl, (Z)-2-methylvinyl, homoallyl, (E)-but-2-enyl, (Z)-but-2-enyl, (E)-but-1-enyl, (Z)-but-1-enyl, pent-4-enyl, (E)-pent-3-enyl, (Z)-pent-3-enyl, (E)-pent-2-enyl, (Z)-pent-2-enyl, (E)-pent-1-enyl, (Z)-pent-1-enyl, hex-5-enyl, (E)-hex-4-enyl, (Z)-hex-4-enyl, (E)-hex-3-enyl, (Z)-hex-3-enyl, (E)-hex-2-enyl, (Z)-hex-2-enyl, (E)-hex-1-enyl, (Z)-hex-1-enyl, iso-propenyl, 2-methyl prop-2-enyl, 1-methyl prop-2-enyl, 2-methyl prop-1-enyl, (E)-1-methyl prop-1-enyl, (Z)-1-methyl prop-1-enyl, 3-methyl but-3-enyl, 2-methyl but-3-enyl, 1-methyl but-3-enyl, 3-methyl but-2-enyl, (E)-2-methyl but-2-enyl, (Z)-2-methyl but-2-enyl, (E)-1-methyl but-2-enyl, (Z)-1-methyl but-2-enyl, (E)-3-methyl but-1-enyl, (Z)-3-methyl but-1-enyl, (E)-2-methyl but-1-enyl, (Z)-2-methyl but-1-enyl, (E)-1-methyl but-1-enyl, (Z)-1-methyl but-1-enyl, 1,1-dimethylprop-2-enyl, 1-ethyl prop-1-enyl, 1-propylvinyl, 1-isopropylvinyl, 4-methyl pent-4-enyl, 3-methyl pent-4-enyl, 2-methyl pent-4-enyl, 1-methyl pent-4-enyl, 4-methyl pent-3-enyl, (E)-3-methyl pent-3-enyl, (Z)-3-methyl pent-3-enyl, (E)-2-methyl pent-3-enyl, (Z)-2-methyl pent-3-enyl, (E)-1-methyl pent-3-enyl, (Z)-1-methyl pent-3-enyl, (E)-4-methyl pent-2-enyl, (Z)-4-methyl pent-2-enyl, (E)-3-methyl pent-2-enyl, (Z)-3-methyl pent-2-enyl, (E)-2-methyl pent-2-enyl, (Z)-2-methyl pent-2-enyl, (E)-1-methyl pent-2-enyl, (Z)-1-methyl pent-2-enyl, (E)-4-methyl pent-1-enyl, (Z)-4-methyl pent-1-enyl, (E)-3-methyl pent-1-enyl, (Z)-3-methyl pent-1-enyl, (E)-2-methyl pent-1-enyl, (Z)-2-methyl pent-1-enyl, (E)-1-methyl pent-1-enyl, (Z)-1-methyl pent-1-enyl, 3-ethyl but-3-enyl, 2-ethyl but-3-enyl, 1-ethyl but-3-enyl, (E)-3-ethyl but-2-enyl, (Z)-3-ethyl but-2-enyl, (E)-2-ethyl but-2-enyl, (Z)-2-ethyl but-2-enyl, (E)-1-ethyl but-2-enyl, (Z)-1-ethyl but-2-enyl, (E)-3-ethyl but-1-enyl, (Z)-3-ethyl but-1-enyl, 2-ethyl but-1-enyl, (E)-1-ethyl but-1-enyl, (Z)-1-ethyl but-1-enyl, 2-propyl prop-2-enyl, 1-propyl prop-2-enyl, 2-isopropyl prop-2-enyl, 1-isopropyl prop-2-enyl, (E)-2-propyl prop-1-enyl, (Z)-2-propyl prop-1-enyl, (E)-1-propyl prop-1-enyl, (Z)-1-propyl prop-1-enyl, (E)-2-isopropyl prop-1-enyl, (Z)-2-isopropyl prop-1-enyl, (E)-1-isopropylprop-1-enyl, (Z)-1-isopropyl prop-1-enyl, (E)-3,3-dimethylprop-1-enyl, (Z)-3,3-dimethyl prop-1-enyl, 1-(1,1-dimethylethyl)ethenyl, buta-1,3-dienyl, penta-1,4-dienyl, hexa-1,5-dienyl, or methylhexadienyl group. Particularly, said group is vinyl or allyl.

The term “C₂-C₆-alkynyl” is to be understood as preferably meaning a linear or branched, monovalent hydrocarbon group which contains one or more triple bonds, and which contains 2, 3, 4, 5 or 6 carbon atoms, particularly 2 or 3 carbon atoms (“C₂-C₃-alkynyl”). Said C₂-C₆-alkynyl group is, for example, ethynyl, prop-1-ynyl, prop-2-ynyl, but-1-ynyl, but-2-ynyl, but-3-ynyl, pent-1-ynyl, pent-2-ynyl, pent-3-ynyl, pent-4-ynyl, hex-1-ynyl, hex-2-ynyl, hex-3-ynyl, hex-4-ynyl, hex-5-ynyl, 1-methyl prop-2-ynyl, 2-methyl but-3-ynyl, 1-methyl but-3-ynyl, 1-methyl but-2-ynyl, 3-methyl but-l-ynyl, 1-ethylprop-2-ynyl, 3-methylpent-4-ynyl, 2-methylpent-4-ynyl, 1-methyl-pent-4-ynyl, 2-methylpent-3-ynyl, 1-methylpent-3-ynyl, 4-methylpent-2-ynyl, 1-methylpent-2-ynyl, 4-methyl pent-1-ynyl, 3-methyl pent-1-ynyl, 2-ethyl but-3-ynyl, 1-ethyl but-3-ynyl, 1-ethyl but-2-ynyl, 1-propylprop-2-ynyl, 1-isopropyl prop-2-ynyl, 2,2-dimethyl but-3-ynyl, 1,1-dimethyl but-3-ynyl, 1,1-dimethylbut-2-ynyl, or 3,3-dimethylbut-1-ynyl group. Particularly, said alkynyl group is ethynyl, prop-1-ynyl, or prop-2-ynyl.

The term “C₃-C₇-cycloalkyl” is to be understood as meaning a saturated, monovalent, monocyclic hydrocarbon ring which contains 3, 4, 5, 6 or 7 carbon atoms. Said C₃-C₇-cycloalkyl group is for example a cyclopropyl, cyclobutyl, cyclopentyl, cyclohexyl or cycloheptyl ring. Particularly, said ring contains 3, 4, 5 or 6 carbon atoms (“C₃-C₆-cycloalkyl”).

The term “C₄-C₈-cycloalkenyl” is to be understood as preferably meaning a monovalent, monocyclic hydrocarbon ring which contains 4, 5, 6, 7 or 8 carbon atoms and one or two double bonds, in conjugation or not, as the size of said cycloalkenyl ring allows. Particularly, said ring contains 4, 5 or 6 carbon atoms (“C₄-C₆-cycloalkenyl”). Said C₄-C₈-cycloalkenyl group is for example a cyclobutenyl, cyclopentenyl, or cyclohexenyl group.

The term “C₃-C₆-cycloalkoxy” is to be understood as meaning a saturated, monovalent, monocyclic group of formula —O—(C₃-C₆-cycloalkyl), in which the term “C₃-C₆-cycloalkyl” is defined supra, e.g. a cyclopropyloxy, cyclobutyloxy, cyclopentyloxy or cyclohexyloxy group.

The term “3- to 10-membered heterocycloalkyl”, is to be understood as meaning a saturated, monovalent, mono- or bicyclic hydrocarbon ring which contains 2, 3, 4, 5, 6, 7, 8 or 9 carbon atoms, and one or more heteroatom-containing groups selected from C(═O), O, S, S(═O), S(═O)₂, NH ; it being possible for said heterocycloalkyl group to be attached to the rest of the molecule via any one of the carbon atoms or, if present, a nitrogen atom.

Particularly, said 3- to 10-membered heterocycloalkyl can contain 2, 3, 4, 5 or 6 carbon atoms, and one or more of the above-mentioned heteroatom-containing groups (a “3- to 7-membered heterocycloalkyl”), more particularly said heterocycloalkyl can contain 4, 5 or 6 carbon atoms, and one or more of the above-mentioned heteroatom-containing groups (a “4- to 6-membered heterocycloalkyl”).

Particularly, without being limited thereto, said heterocycloalkyl can be a 4-membered ring, such as an azetidinyl, oxetanyl, or a 5-membered ring, such as tetrahydrofuranyl, dioxolinyl, pyrrolidinyl, imidazolidinyl, pyrazolidinyl, pyrrolinyl, or a 6-membered ring, such as tetrahydropyranyl, piperidinyl, morpholinyl, dithianyl, thiomorpholinyl, piperazinyl, or trithianyl, or a 7-membered ring, such as a diazepanyl ring, for example.

The term “4- to 10-membered heterocycloalkenyl”, is to be understood as meaning an unsaturated, monovalent, mono- or bicyclic hydrocarbon ring which contains 3, 4, 5, 6, 7, 8 or 9 carbon atoms, and one or more heteroatom-containing groups selected from C(═O), O, S, S(═O), S(═O)₂, NH ; it being possible for said heterocycloalkenyl group to be attached to the rest of the molecule via any one of the carbon atoms or, if present, a nitrogen atom. Examples of said heterocycloalkenyl may contain one or more double bonds, e.g. 4H-pyranyl, 2H-pyranyl, 2,5-dihydro-1H-pyrrolyl, [1,3]dioxolyl, 4H-[1,3,4]thiadiazinyl, 2,5-dihydrofuranyl, 2,3-dihydrofuranyl, 2,5-dihydrothiophenyl, 2,3-dihydrothiophenyl, 4,5-dihydrooxazolyl, or 4H-[1,4]thiazinyl group.

The term “aryl” is to be understood as preferably meaning a monovalent, aromatic or partially aromatic, mono-, or bi- or tricyclic hydrocarbon ring having 6, 7, 8, 9, 10, 11, 12, 13 or 14 carbon atoms (a “C₆-C₁₄-aryl” group), particularly a ring having 6 carbon atoms (a “C₆-aryl” group), e.g. a phenyl group; or a ring having 9 carbon atoms (a “C₉-aryl” group), e.g. an indanyl or indenyl group, or a ring having 10 carbon atoms (a “C₁₀-aryl” group), e.g. a tetralinyl, dihydronaphthyl, or naphthyl group, or a biphenyl group (a “C₁₂-aryl” group), or a ring having 13 carbon atoms, (a “C₁₃-aryl” group), e.g. a fluorenyl group, or a ring having 14 carbon atoms, (a “C₁₄-aryl” group), e.g. an anthracenyl group. Preferably, the aryl group is a phenyl group.

The term “heteroaryl” is understood as preferably meaning a monovalent, monocyclic- , bicyclic- or tricyclic aromatic ring system having 5, 6, 7, 8, 9, 10, 11, 12, 13 or 14 ring atoms (a “5- to 14-membered heteroaryl” group), particularly 5 or 6 or 9 or 10 atoms, and which contains at least one heteroatom which may be identical or different, said heteroatom being such as oxygen, nitrogen or sulfur, and in addition in each case can be benzocondensed. Particularly, heteroaryl is selected from thienyl, furanyl, pyrrolyl, oxazolyl, thiazolyl, imidazolyl, pyrazolyl, isoxazolyl, isothiazolyl, oxadiazolyl, triazolyl, thiadiazolyl, thia-4H-pyrazolyl etc., and benzo derivatives thereof, such as, for example, benzofuranyl, benzothienyl, benzoxazolyl, benzisoxazolyl, benzimidazolyl, benzotriazolyl, indazolyl, indolyl, isoindolyl, etc.; or pyridinyl, pyridazinyl, pyrimidinyl, pyrazinyl, triazinyl, etc., and benzo derivatives thereof, such as, for example, quinolinyl, quinazolinyl, isoquinolinyl, etc.; or azocinyl, indolizinyl, purinyl, etc., and benzo derivatives thereof; or cinnolinyl, phthalazinyl, quinazolinyl, quinoxalinyl, naphthpyridinyl, pteridinyl, carbazolyl, acridinyl, phenazinyl, phenothiazinyl, phenoxazinyl, xanthenyl, or oxepinyl, etc..

In general, and unless otherwise mentioned, the heteroarylic or heteroarylenic radicals include all the possible isomeric forms thereof, e.g. the positional isomers thereof. Thus, for some illustrative non-restricting example, the term pyridyl includes pyridin-2-yl, pyridin-3-yl, and pyridin-4-yl; or the term thienyl includes thien-2-yl and thien-3-yl. Preferably, the heteroaryl group is a pyridinyl group.

The term “C₁-C₆”, as used throughout this text, e.g. in the context of the definition of “C₁-C₆-alkyl”, “C₁-C₆-haloalkyl”, “C₁-C₆-alkoxy”, or “C₁-C₆-haloalkoxy” is to be understood as meaning an alkyl group having a finite number of carbon atoms of 1 to 6, i.e. 1, 2, 3, 4, 5, or 6 carbon atoms. It is to be understood further that said term “C₁-C₆” is to be interpreted as any sub-range comprised therein, e.g. C₁-C₆, C₂-C₅, C₃-C₄, C₁-C₂, C₁-C₃, C₁-C₄, C₁-C₅, C₁-C₆, particularly C₁-C₂,C₁-C₃, C₁-C₄, C₁-C₅, C₁-C₆, more particularly C₁-C₄; in the case of “C₁-C₆-haloalkyl” or “C₁-C₆-haloalkoxy” even more particularly C₁-C₂.

Similarly, as used herein, the term “C₂-C₆”, as used throughout this text, e.g. in the context of the definitions of “C₂-C₆-alkenyl” and “C₂-C₆-alkynyl”, is to be understood as meaning an alkenyl group or an alkynyl group having a finite number of carbon atoms of 2 to 6, i.e. 2, 3, 4, 5, or 6 carbon atoms. It is to be understood further that said term “C₂-C₆” is to be interpreted as any sub-range comprised therein, e.g. C₂-C₆, C₃-C₅, C₃-C₄, C₂-C₃, C₂-C₄, C₂-C₅; particularly C₂-C₃.

Further, as used herein, the term “C₃-C₇”, as used throughout this text, e.g. in the context of the definition of “C₃-C₇-cycloalkyl”, is to be understood as meaning a cycloalkyl group having a finite number of carbon atoms of 3 to 7, i.e. 3, 4, 5, 6 or 7 carbon atoms. It is to be understood further that said term “C₃-C₇” is to be interpreted as any sub-range comprised therein, e.g. C₃-C₆, C₄-C₅, C₃-C₅, C₃-C₄, C₄-C₆, C₅-C₇; particularly C₃-C₆.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds.

The term “optionally substituted” means that the number of substituents can be zero. Unless otherwise indicated, optionally substituted groups may be substituted with as many optional substituents as can be accommodated by replacing a hydrogen atom with a non-hydrogen substituent on any available carbon or nitrogen atom. Commonly, the number of optional substituents (when present) ranges from 1 to 3.

Ring system substituent means a substituent attached to an aromatic or nonaromatic ring system which, for example, replaces an available hydrogen on the ring system.

As used herein, the term “one or more times”, e.g. in the definition of the substituents of the compounds of the general formulae of the present invention, is understood as meaning “one, two, three, four or five times, particularly one, two, three or four times, more particularly one, two or three times, even more particularly one or two times”.

As used herein, the term “leaving group” refers to an atom or a group of atoms that is displaced in a chemical reaction as stable species taking with it the bonding electrons. Preferably, a leaving group is selected from the group comprising: halo, in particular chloro, bromo or iodo, methanesulfonyloxy, p-toluenesulfonyloxy, trifluoromethanesulfonyloxy, nonafluorobutanesulfonyloxy, (4-bromo-benzene)sulfonyloxy, (4-nitro-benzene)sulfonyloxy, (2-nitro-benzene)-sulfonyloxy, (4-isopropyl-benzene)sulfonyloxy, (2,4,6-tri-isopropyl-benzene)-sulfonyloxy, (2,4,6-trimethyl-benzene)sulfonyloxy, (4-tertbutyl-benzene)sulfonyloxy, benzenesulfonyloxy, and (4-methoxy-benzene)sulfonyloxy.

Where the plural form of the word compounds, salts, polymorphs, hydrates, solvates and the like, is used herein, this is taken to mean also a single compound, salt, polymorph, isomer, hydrate, solvate or the like.

The compounds of this invention contain one or more asymmetric centres, depending upon the location and nature of the various substituents desired. Asymmetric carbon atoms may be present in the (R) or (S) configuration. In certain instances, asymmetry may also be present due to restricted rotation about a given bond, for example, the central bond adjoining two substituted aromatic rings of the specified compounds.

Substituents on a ring may also be present in either cis or trans form. It is intended that all such configurations are included within the scope of the present invention.

Preferred compounds are those which produce the more desirable biological activity. Separated, pure or partially purified isomers and stereoisomers or racemic or diastereomeric mixtures of the compounds of this invention are also included within the scope of the present invention. The purification and the separation of such materials can be accomplished by standard techniques known in the art.

The optical isomers can be obtained by resolution of the racemic mixtures according to conventional processes, for example, by the formation of diastereoisomeric salts using an optically active acid or base or formation of covalent diastereomers. Examples of appropriate acids are tartaric, diacetyltartaric, ditoluoyltartaric and camphorsulfonic acid. Mixtures of diastereoisomers can be separated into their individual diastereomers on the basis of their physical and/or chemical differences by methods known in the art, for example, by chromatography or fractional crystallisation. The optically active bases or acids are then liberated from the separated diastereomeric salts. A different process for separation of optical isomers involves the use of chiral chromatography (e.g., chiral HPLC columns), with or without conventional derivatisation, optimally chosen to maximise the separation of the enantiomers. Suitable chiral HPLC columns are manufactured by Diacel, e.g., Chiracel OD and Chiracel OJ among many others, all routinely selectable. Enzymatic separations, with or without derivatisation, are also useful. The optically active compounds of this invention can likewise be obtained by chiral syntheses utilizing optically active starting materials.

In order to limit different types of isomers from each other reference is made to IUPAC Rules Section E (Pure Appl Chem 45, 11-30, 1976).

The invention also includes all suitable isotopic variations of a compound of the invention. An isotopic variation of a compound of the invention is defined as one in which at least one atom is replaced by an atom having the same atomic number but an atomic mass different from the atomic mass usually or predominantly found in nature. Examples of isotopes that can be incorporated into a compound of the invention include isotopes of hydrogen, carbon, nitrogen, oxygen, phosphorus, sulphur, fluorine, chlorine, bromine and iodine, such as ²H (deuterium), ³H (tritium), ¹¹C, ¹³C, ¹⁴C, ¹⁵N, ¹⁷O, ¹⁸O, ³²P, ³³P, ³³S, ³⁴S, ³⁵S , ³⁶S, ¹⁸F, ³⁶Cl, ₈₂Br, ¹²³I, ¹²⁴I, ¹²⁹I, and ¹³¹I, respectively. Certain isotopic variations of a compound of the invention, for example, those in which one or more radioactive isotopes such as ³H or ¹⁴C are incorporated, are useful in drug and/or substrate tissue distribution studies. Tritiated and carbon-14, i.e., ¹⁴C, isotopes are particularly preferred for their ease of preparation and detectability. Further, substitution with isotopes such as deuterium may afford certain therapeutic advantages resulting from greater metabolic stability, for example, increased in vivo half-life or reduced dosage requirements and hence may be preferred in some circumstances. Isotopic variations of a compound of the invention can generally be prepared by conventional procedures known by a person skilled in the art such as by the illustrative methods or by the preparations described in the examples hereafter using appropriate isotopic variations of suitable reagents.

The present invention includes all possible stereoisomers of the compounds of the present invention as single stereoisomers, or as any mixture of said stereoisomers, in any ratio. Isolation of a single stereoisomer, e.g. a single enantiomer or a single diastereomer, of a compound of the present invention may be achieved by any suitable state of the art method, such as chromatography, especially chiral chromatography, for example.

Further, the compounds of the present invention may exist as tautomers. For example, any compound of the present invention which contains a pyrazole moiety as a heteroaryl group for example can exist as a 1H tautomer, or a 2H tautomer, or even a mixture in any amount of the two tautomers, or a triazole moiety for example can exist as a 1H tautomer, a 2H tautomer, or a 4H tautomer, or even a mixture in any amount of said 1H, 2H and 4H tautomers, viz.:

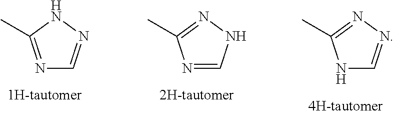

The present invention includes all possible tautomers of the compounds of the present invention as single tautomers, or as any mixture of said tautomers, in any ratio.

Further, the compounds of the present invention can exist as N-oxides, which are defined in that at least one nitrogen of the compounds of the present invention is oxidised. The present invention includes all such possible N-oxides.

The present invention also relates to useful forms of the compounds as disclosed herein, such as metabolites, hydrates, solvates, prodrugs, salts, in particular pharmaceutically acceptable salts, and co-precipitates.

The compounds of the present invention can exist as a hydrate, or as a solvate, wherein the compounds of the present invention contain polar solvents, in particular water, methanol or ethanol for example as structural element of the crystal lattice of the compounds. The amount of polar solvents, in particular water, may exist in a stoichiometric or non-stoichiometric ratio. In the case of stoichiometric solvates, e.g. a hydrate, hemi-, (semi-), mono-, sesqui-, di-, tri-, tetra-, penta- etc. solvates or hydrates, respectively, are possible. The present invention includes all such hydrates or solvates.

Further, the compounds of the present invention can exist in free form, e.g. as a free base, or as a free acid, or as a zwitterion, or can exist in the form of a salt. Said salt may be any salt, either an organic or inorganic addition salt, particularly any pharmaceutically acceptable organic or inorganic addition salt, customarily used in pharmacy.

The present invention includes all possible salts of the compounds of the present invention as single salts, or as any mixture of said salts, in any ratio.

Furthermore, the present invention includes all possible crystalline forms, or polymorphs, of the compounds of the present invention, either as single polymorphs, or as a mixture of more than one polymorphs, in any ratio.

In accordance with a first aspect, the present invention covers compounds of general formula (I):

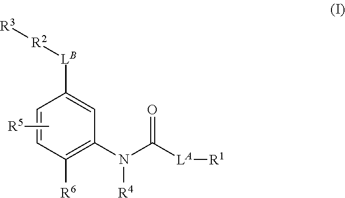

in which:
L^{A} represents
   *CH₂**;
   wherein * indicates the point of attachment to the carbonyl group, and ** indicates the point of attachment to R¹;
L^{B} represents *N(H)—C(═O)**;
   wherein * indicates the point of attachment to R², and ** indicates the point of attachment to the phenyl group;
R¹ represents a group selected from:

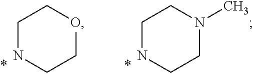

wherein * indicates the point of attachment to L^{A},
R² represents

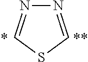

wherein * indicates the point of attachment to R³, and ** indicates the point of attachment to L^{B};
R³ represents a group selected from:

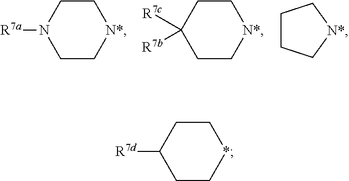

wherein * indicates the point of attachment to R²;
R⁴ represents a hydrogen atom;
R⁵ represents a hydrogen atom;
R⁶ represents a —O—CF₃ group;
R^{7a} represents a group selected from:
   —C(═O)—R⁸, —C(═O)—O—R⁸, —C(═O)—N(R⁸)(R⁹), —S(═O)₂—(R⁸)(R⁹),
R^{7b} represents a hydrogen atom or a methyl-group;
R^{7c} represents a hydrogen atom or a group selected from:
   methyl-, —OH, HO—(C₁-C₃-alkyl)-, methoxy-, —C(═O)—O—R⁸;
R^{7d} represents a hydrogen atom;
R⁸ represents a group selected from:
   —CH₃, —CH₂-CH₃, —C(H)(CH₃)₂, —C(CH₃)₃, -cyclopropyl;
R⁹ represents a —CH₃ group;

or a tautomer, an N-oxide, a hydrate, a solvate, or a salt thereof, or a mixture of same.

In a preferred embodiment, the present invention relates to compounds of the general formula (I), supra, in which R¹ represents

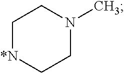

wherein * indicates the point of attachment to L^{A}.

In another preferred embodiment, the present invention relates to compounds of the general formula (I), supra, in which R³ represents

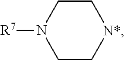

wherein * indicates the point of attachment to R².

In another preferred embodiment, the present invention relates to compounds of the general formula (I), supra, in which R³ is selected from:

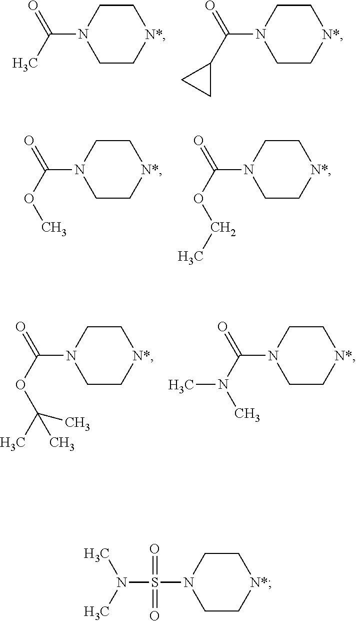

wherein * indicates the point of attachment to R².

In another preferred embodiment, the present invention relates to compounds of the general formula (I), supra, in which R³ represents

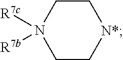

wherein * indicates the point of attachment to R².

In another preferred embodiment, the present invention relates to compounds of the general formula (I), supra, in which R³ is selected from:

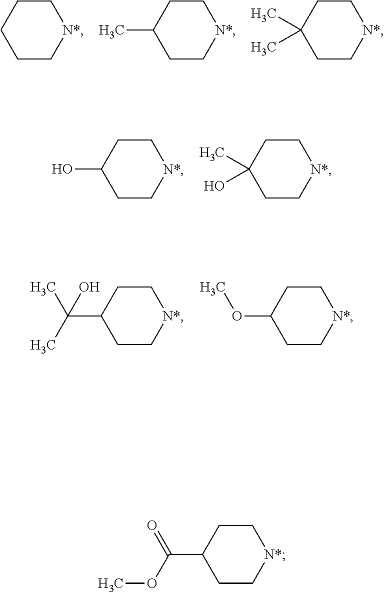

; wherein * indicates the point of attachment to R².

In another preferred embodiment, the present invention relates to compounds of the general formula (I), supra, in which R³ represents

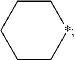

wherein * indicates the point of attachment to R².

In another preferred embodiment, the present invention relates to compounds of the general formula (I), supra, in which R^{7a} represents —C(═O)—R⁸.

In another preferred embodiment, the present invention relates to compounds of the general formula (I), supra, in which R^{7a} represents —C(═O)—O—R⁸.

In another preferred embodiment, the present invention relates to compounds of the general formula (I), supra, in which R^{7a} represents —C(═O)—N(R⁸)(R⁹).

In another preferred embodiment, the present invention relates to compounds of the general formula (I), supra, in which R^{7a} represents —S(═O)₂—N(R⁸)(R⁹).

In another preferred embodiment, the present invention relates to compounds of the general formula (I), supra, in which R^{7b} represents a hydrogen atom.

In another preferred embodiment, the present invention relates to compounds of the general formula (I), supra, in which R^{7b} represents a methyl-group.

In another preferred embodiment, the present invention relates to compounds of the general formula (I), supra, in which R^{7c} represents a hydrogen atom.

In another preferred embodiment, the present invention relates to compounds of the general formula (I), supra, in which R^{7c} represents a methyl-group.

In another preferred embodiment, the present invention relates to compounds of the general formula (I), supra, in which R^{7c} represents a methoxy-group.

In another preferred embodiment, the present invention relates to compounds of the general formula (I), supra, in which R^{7c} represents a —OH group.

In another preferred embodiment, the present invention relates to compounds of the general formula (I), supra, in which R^{7c} represents a HO—(C₁-C₃-alkyl)-group.

In another preferred embodiment, the present invention relates to compounds of the general formula (I), supra, in which R^{7c} represents a —C(═O)—O—R⁸ group.

In another preferred embodiment, the present invention relates to compounds of the general formula (I), supra, in which R⁸ represents —CH₃.

In another preferred embodiment, the present invention relates to compounds of the general formula (I), supra, in which R⁸ represents —CH₂-CH₃.

In another preferred embodiment, the present invention relates to compounds of the general formula (I), supra, in which R⁸ represents —C(CH₃)₃.

In another preferred embodiment, the present invention relates to compounds of the general formula (I), supra, in which R⁸ represents cyclopropyl.

It is to be understood that the present invention relates also to any combination of the preferred embodiments described above.

Some examples of combinations are given hereinafter. However, the invention is not limited to these combinations.

In a preferred embodiment, the present invention relates to compounds of the general formula (I),

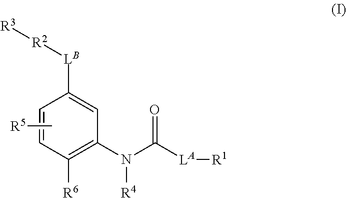

in which:
L^{A} represents

*CH₂**;

wherein * indicates the point of attachment to the carbonyl group, and ** indicates the point of attachment to R¹;
L^{B} represents *N(H)—C(═O)**;

wherein * indicates the point of attachment to R², and ** indicates the point of attachment to the phenyl group;
R¹ represents

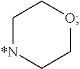

wherein * indicates the point of attachment to L^{A},
R² represents

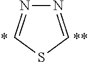

wherein * indicates the point of attachment to R³, and ** indicates the point of attachment to L^{B};
R³ is selected from:

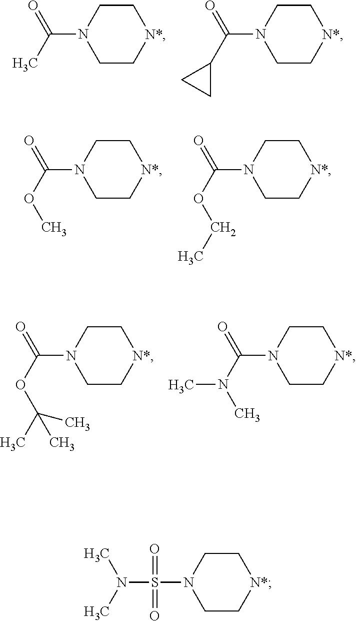

wherein * indicates the point of attachment to R²;
R⁴ represents a hydrogen atom;
R⁵ represents a hydrogen atom;
R⁶ represents a —O—CF₃ group;

or a tautomer, an N-oxide, a hydrate, a solvate, or a salt thereof, or a mixture of same.

In another preferred embodiment, the present invention relates to compounds of the general formula (I),

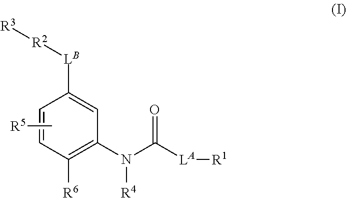

in which :
L^{A} represents

*CH₂**;

wherein * indicates the point of attachment to the carbonyl group, and ** indicates the point of attachment to R¹;
L^{B} represents *N(H)13 C(═O)**;

wherein * indicates the point of attachment to R², and ** indicates the point of attachment to the phenyl group;
R¹ represents

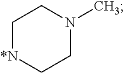

wherein * indicates the point of attachment to L^{A} ;
R² represents

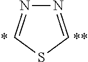

wherein * indicates the point of attachment to R³, and ** indicates the point of attachment to L^{B};
R³ is selected from:

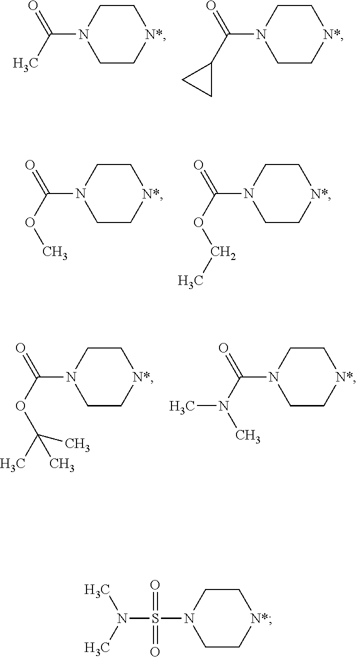

wherein * indicates the point of attachment to R²;
R⁴ represents a hydrogen atom;
R⁵ represents a hydrogen atom;
R⁶ represents a —O—CF₃ group;

or a tautomer, an N-oxide, a hydrate, a solvate, or a salt thereof, or a mixture of same.

In another preferred embodiment, the present invention relates to compounds of the general formula (I),

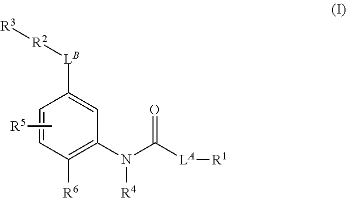

in which :
L^{A} represents

*CH₂**;

wherein * indicates the point of attachment to the carbonyl group, and ** indicates the point of attachment to R¹;
L^{B} represents *N(H)—C(═O)**;

wherein * indicates the point of attachment to R², and ** indicates the point of attachment to the phenyl group;

R¹ represents

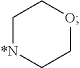

wherein * indicates the point of attachment to L^{A},
R² represents

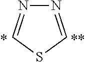

wherein * indicates the point of attachment to R³, and ** indicates the point of attachment to L^{B};
R³ is selected from:

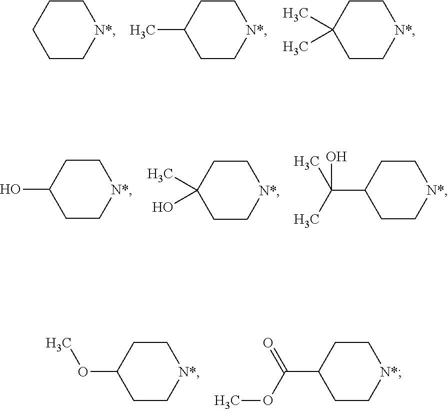

wherein * indicates the point of attachment to R²;
R⁴ represents a hydrogen atom;
R⁵ represents a hydrogen atom;
R⁶ represents a —O—CF₃ group;

or a tautomer, an N-oxide, a hydrate, a solvate, or a salt thereof, or a mixture of same.

In another preferred embodiment, the present invention relates to compounds of the general formula (I),

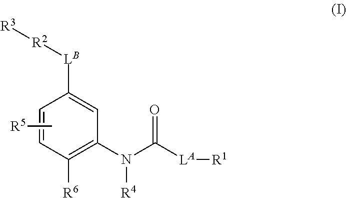

in which :
L^{A} represents
   *CH₂**;
      wherein * indicates the point of attachment to the carbonyl group, and ** indicates the point of attachment to R¹;
L^{B} represents *N(H)—C(═O)**;

wherein * indicates the point of attachment to R², and ** indicates the point of attachment to the phenyl group;
R¹ represents

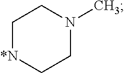

wherein * indicates the point of attachment to L^{A} ;
R² represents

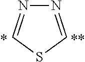

wherein * indicates the point of attachment to R³, and ** indicates the point of attachment to L^{B};
R³ is selected from:

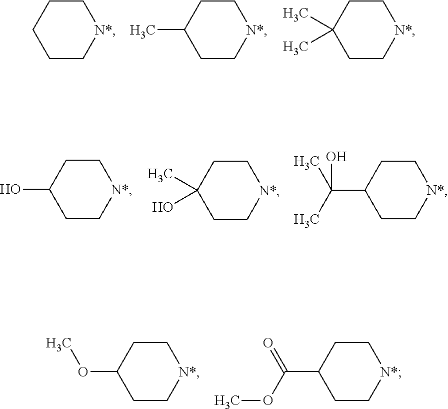

wherein * indicates the point of attachment to R²;
R⁴ represents a hydrogen atom;
R⁵ represents a hydrogen atom;
R⁶ represents a —O—CF₃ group;

or a tautomer, an N-oxide, a hydrate, a solvate, or a salt thereof, or a mixture of same.

In another preferred embodiment, the present invention relates to compounds of the general formula (I),

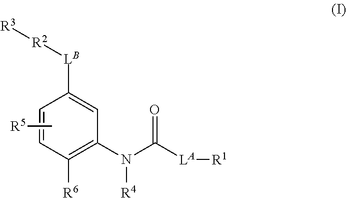

in which :
L^{A} represents

*CH₂**;

wherein * indicates the point of attachment to the carbonyl group, and ** indicates the point of attachment to R¹;
L^{B} represents *N(H)—C(═O)**;

wherein * indicates the point of attachment to R², and ** indicates the point of attachment to the phenyl group;
R¹ represents

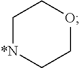

wherein * indicates the point of attachment to L^{A},
R² represents

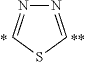

wherein * indicates the point of attachment to R³, and ** indicates the point of attachment to L^{B};
R³ is selected from:

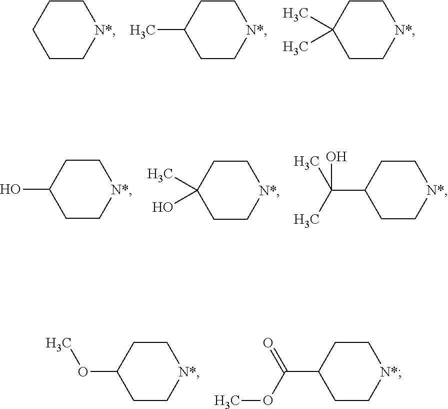

wherein * indicates the point of attachment to R²;
R⁴ represents a hydrogen atom;
R⁵ represents a hydrogen atom;
R⁶ represents a —O—CF₃ group;

or a tautomer, an N-oxide, a hydrate, a solvate, or a salt thereof, or a mixture of same.

In another preferred embodiment, the present invention relates to compounds of the general formula (I),

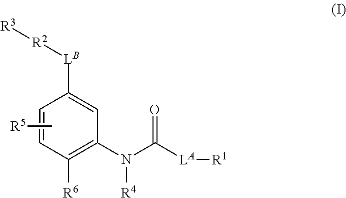

in which:
L^{A} represents

*CH₂**;

wherein * indicates the point of attachment to the carbonyl group, and ** indicates the point of attachment to R¹;
L^{B} represents *N(H)—C(═O)**;

wherein * indicates the point of attachment to R², and ** indicates the point of attachment to the phenyl group;
R¹ represents

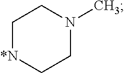

wherein * indicates the point of attachment to L^{A} ;
R² represents

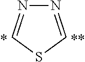

wherein * indicates the point of attachment to R³, and ** indicates the point of attachment to L^{B};
R³ is selected from:

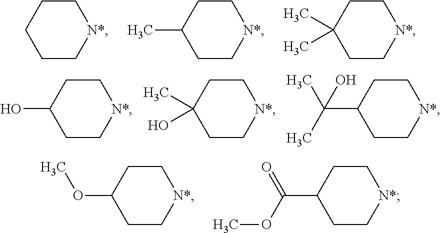

wherein * indicates the point of attachment to R²;
R⁴ represents a hydrogen atom;
R⁵ represents a hydrogen atom;
R⁶ represents a —O—CF₃ group;

or a tautomer, an N-oxide, a hydrate, a solvate, or a salt thereof, or a mixture of same.

In another preferred embodiment, the present invention relates to compounds of the general formula (I),

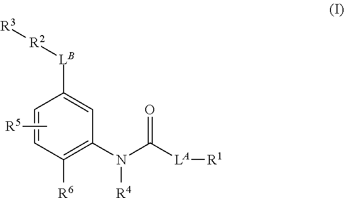

in which :
L^{A} represents

*CH₂**;

wherein * indicates the point of attachment to the carbonyl group, and ** indicates the point of attachment to R¹;
L^{B} represents *N(H)—C(═O)**;

wherein * indicates the point of attachment to R², and ** indicates the point of attachment to the phenyl group;
R¹ represents

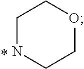

wherein * indicates the point of attachment to L^{A},
R² represents

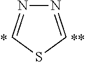

wherein * indicates the point of attachment to R³, and ** indicates the point of attachment to L^{B};
R³ represents

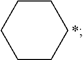

wherein * indicates the point of attachment to R²;
R⁴ represents a hydrogen atom;
R⁵ represents a hydrogen atom;
R⁶ represents a —O—CF₃ group;

or a tautomer, an N-oxide, a hydrate, a solvate, or a salt thereof, or a mixture of same.

In another preferred embodiment, the present invention relates to compounds of the general formula (I),

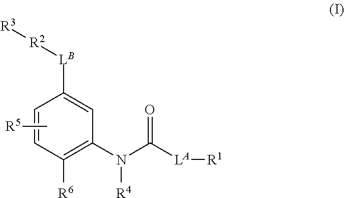

in which:
L^{A} represents

*CH₂**;

wherein * indicates the point of attachment to the carbonyl group, and ** indicates the point of attachment to R¹;
L^{B} represents *N(H)—C(═O)**;

wherein * indicates the point of attachment to R², and ** indicates the point of attachment to the phenyl group;
R¹ represents

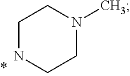

wherein * indicates the point of attachment to L^{A};
R² represents

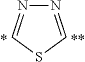

wherein * indicates the point of attachment to R³, and ** indicates the point of attachment to L^{B};
R³ represents

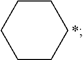

wherein * indicates the point of attachment to R²;
R⁴ represents a hydrogen atom;
R⁵ represents a hydrogen atom;
R⁶ represents a —O—CF₃ group;

or a tautomer, an N-oxide, a hydrate, a solvate, or a salt thereof, or a mixture of same.

It is to be understood that the present invention relates also to any combination of the preferred embodiments described above.

More particularly still, the present invention covers compounds of general formula (I) which are disclosed in the Examples section of this text, infra.

In accordance with another aspect, the present invention covers methods of preparing compounds of the present invention, said methods comprising the steps as described in the Experimental Section herein.

In a preferred embodiment, the present invention relates to a method of preparing a compound of general formula (I), supra, said method comprising the step of allowing an intermediate compound of general formula (VI):

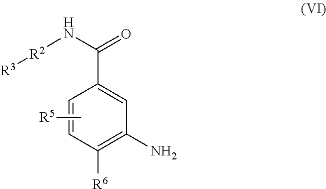

in which R², R³, R⁵, and R⁶ are as defined for general formula (I), supra; to react with a carboxylic acid HO₂C-L^{A}-R¹ or the corresponding acyl chloride CI—C(═O)-L^{A}-R¹, wherein

L^{A} and R¹ are as defined for the compounds of general formula (I), supra; or alternatively to react with suitable reagents, such as Cl—C(═O) -L^{A}-LG, in which L^{A} is as defined for the compounds of general formula (I), and LG stands for a leaving group, preferably chloro or bromo, and subsequently with agents suitable for the introduction of R¹, exemplified by but not limited to cyclic secondary amines;

thereby giving, upon optional deprotection, a compound of general formula (la):

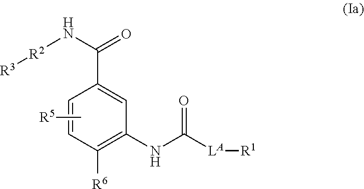

in which L^{A}, R¹, R², R³, R⁵, and R⁶ are as defined for the compounds of general formula (I), supra.

In accordance with another embodiment, the present invention also relates to a method of preparing a compound of general formula (I), supra, said method comprising the step of allowing an intermediate compound of general formula (XI):

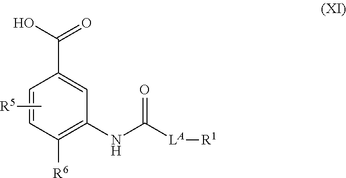

in which L^{A}, R¹, R⁵, and R⁶ are as defined for general formula (I), supra; to react with a compound of general formula R³R²NH₂, in which R² and R³ are as defined for the compounds of general formula (I), supra;

thereby giving, upon optional deprotection, a compound of general formula (la):

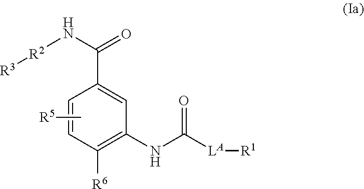

in which L^{A}, R¹, R², R³, R⁵, and R⁶ are as defined for the compounds of general formula (I), supra.

In accordance with another embodiment, the present invention also relates to a method of preparing a compound of general formula (I), supra, said method comprising the step of allowing an intermediate compound of general formula (Xla):

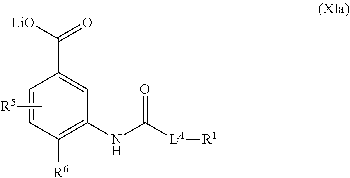

in which L^{A}, R¹, R⁵, and R⁶ are as defined for general formula (I), supra;

to react with a compound of general formula R³R²NH₂, in which R² and R³ are as defined for the compounds of general formula (I), supra;

thereby giving, upon optional deprotection, a compound of general formula (la):

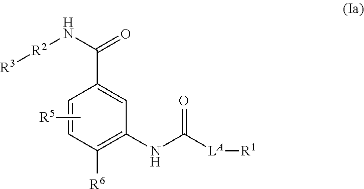

in which L^{A}, R¹, R², R³, R⁵, and R⁶ are as defined for the compounds of general formula (I), supra.

In accordance with another embodiment, the present invention also relates to a method of preparing a compound of general formula (I), supra, said method comprising the step of allowing an intermediate compound of general formula (XVII):

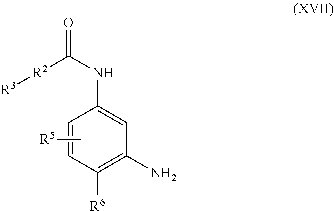

in which R², R³, R⁵, and R⁶ are as defined for general formula (I), supra;

to react with a carboxylic acid HO₂C-L^{A}-R¹ or the corresponding acyl chloride CI—C(═O)-L^{A}-R¹, wherein L^{A} and R¹ are as defined for the compounds of general formula (I), supra; or alternatively to react with suitable reagents, such as Cl—C(═O)-L^{A}-LG, in which L^{A} is as defined for the compounds of general formula (I), and LG stands for a leaving group, preferably chloro or bromo, and subsequently with agents suitable for the introduction of R¹, exemplified by but not limited to cyclic secondary amines;

thereby giving, upon optional deprotection, a compound of general formula (Ib):

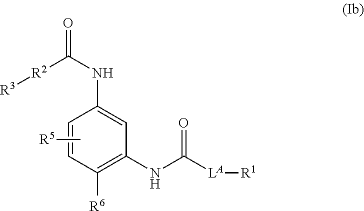

in which L^{A}, R¹, R², R³, R⁵, and R⁶ are as defined for the compounds of general formula (I), supra.

In accordance with another embodiment, the present invention also relates to a method of preparing a compound of general formula (I), supra, said method comprising the step of allowing an intermediate compound of general formula (XXII):

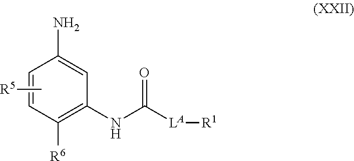

in which L^{A}, R¹, R⁵ and R⁶ are as defined for general formula (I), supra;

to react with a carboxylic acid HO₂C—R²—R³, wherein R² and R³ are as defined for the compounds of general formula (I), supra; or alternatively

to react with a carboxylic acid X—R²—CO₂H, in which R² is as defined for the compounds of general formula (I), supra, and subsequently subjected to a palladium catalysed coupling reaction, such as a Suzuki coupling, with R³—X′, in which R³ is as defined for the compounds of general formula (I), supra. In X—R²—CO₂H and R³—X′, both X and X′ represent groups enabling palladium catalysed coupling reactions, such as chloro, bromo, iodo, trifluoromethylsulfonyloxy, nonafluorobutylsulfonyloxy or a boronic acid or an ester thereof, with the proviso that if X represents a boronic acid or an ester thereof, X′ stands for chloro, bromo, iodo, trifluoromethylsulfonyloxy or nonafluorobutylsulfonyloxy and the like, or vice versa;

thereby giving, upon optional deprotection, a compound of general formula (Ib):

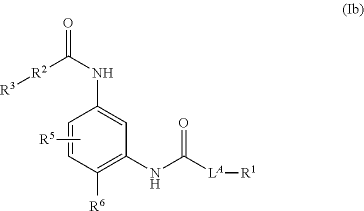

in which L^{A}, R¹, R², R³, R⁵, and R⁶ are as defined for the compounds of general formula (I), supra.

In accordance with another embodiment, the present invention also relates to a method of preparing a compound of general formula (I), supra, said method comprising the step of allowing an intermediate compound of general formula (XXIV):

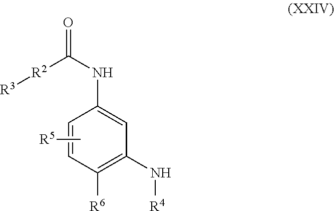

in which R², R³, R⁴, R⁵ and R⁶ are as defined for general formula (I), supra; to react with a carboxylic acid HO₂C-L^{A}-R¹ or the corresponding acyl chloride Cl—C(═O)-L^{A}-R¹, wherein L^{A} and R¹ are as defined for the compounds of general formula (I), supra;

thereby giving, upon optional deprotection, a compound of general formula (Ic):

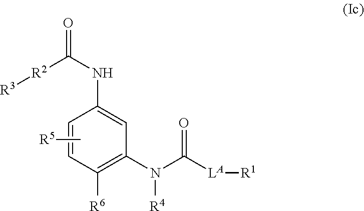

in which L^{A}, R¹, R², R³, R⁴, R⁵ and R⁶ are as defined for the compounds of general formula (I), supra.

In accordance with another embodiment, the present invention also relates to a method of preparing a compound of general formula (I), supra, said method comprising the step of allowing an intermediate compound of general formula (XXV):

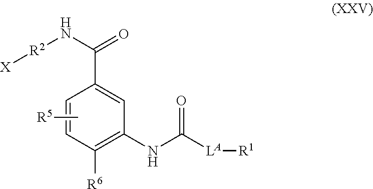

in which L^{A}, R¹, R², R⁵ and R⁶ are as defined for general formula (I), supra;

to react with a compound of general formula R³—X′, wherein R³ is as defined for the compounds of general formula (I), supra;

wherein both, X and X′ represent groups enabling palladium catalysed coupling reactions, such as chloro, bromo, iodo, trifluoromethylsulfonyloxy, nonafluorobutylsulfonyloxy or a boronic acid or an ester thereof, with the proviso that if X represents a boronic acid or an ester thereof, X′ stands for chloro, bromo, iodo, trifluoromethylsulfonyloxy or nonafluorobutylsulfonyloxy and the like, or vice versa.

thereby giving, upon optional deprotection, a compound of general formula (la):

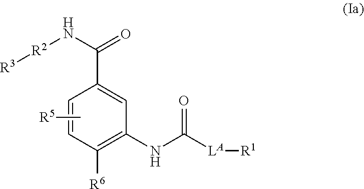

in which L^{A}, R¹, R², R³, R⁴, R⁵ and R⁶ are as defined for the compounds of general formula (I), supra.

In accordance with a further aspect, the present invention covers intermediate compounds which are useful in the preparation of compounds of the present invention of general formula (I), particularly in the method described herein. In particular, the present invention covers intermediate compounds of general formula (VI):

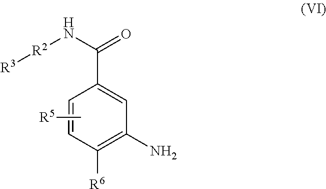

in which R², R³, R⁵, and R⁶ are as defined for general formula (I), supra.

The present invention also covers intermediate compounds of general formula (XI):

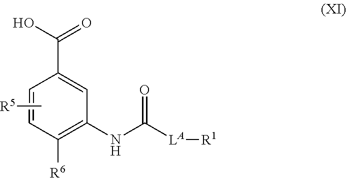

in which L^{A}, R¹, R⁵, and R⁶ are as defined for the compounds of general formula (I), supra.

The present invention also covers intermediate compounds of general formula (Xla):

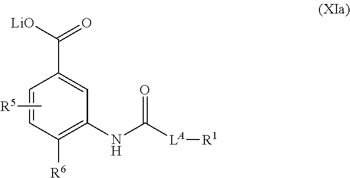

in which L^{A}, R¹, R⁵, and R⁶ are as defined for general formula (I), supra.

The present invention also covers intermediate compounds of general formula (XVII):

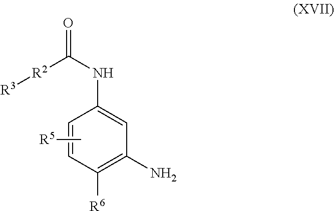

in which R², R³, R⁵, and R⁶ are as defined for general formula (I), supra.

The present invention also covers intermediate compounds of general formula (XXII):

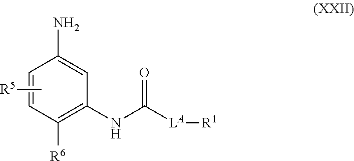

in which L^{A}, R¹, R⁵ and R⁶ are as defined for general formula (I), supra.

The present invention also covers intermediate compounds of general formula (XXIV):

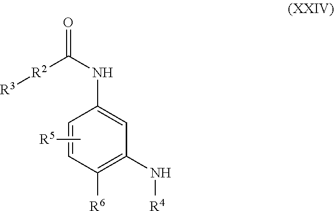

in which R², R³, R⁴, R⁵ and R⁶ are as defined for general formula (I), supra.

The present invention also covers intermediate compounds of general formula (XXV):

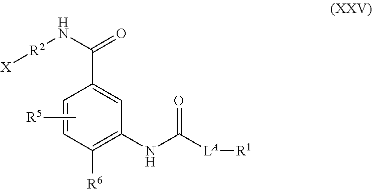

in which L^{A}, R¹, R², R⁵ and R⁶ are as defined for general formula (I), supra, and X represents a group enabling palladium catalysed coupling reactions, such as chloro, bromo, iodo, trifluoromethylsulfonyloxy, nonafluorobutylsulfonyloxy or a boronic acid or an ester thereof.

In accordance with yet another aspect, the present invention covers the use of the intermediate compounds of general formula (VI):

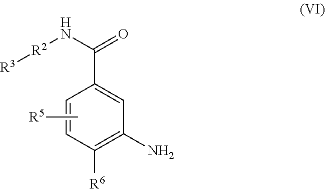

in which R², R³, R⁵, and R⁶ are as defined for general formula (I) supra, for the preparation of a compound of general formula (I) as defined supra.

In accordance with yet another aspect, the present invention covers the use of the intermediate compounds of general formula (XI):

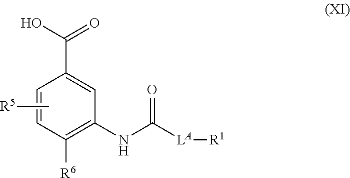

in which L^{A}, R¹, R⁵, and R⁶ are as defined for the compounds of general formula (I) supra, for the preparation of a compound of general formula (I) as defined supra.

In accordance with yet another aspect, the present invention covers the use of the intermediate compounds of general formula (Xla):

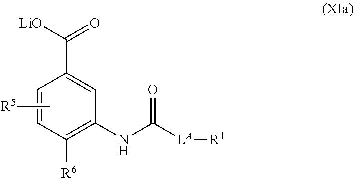

in which L^{A}, R¹, R⁵, and R⁶ are as defined for general formula (I) supra, for the preparation of a compound of general formula (I) as defined supra.

In accordance with yet another aspect, the present invention covers the use of the intermediate compounds of general formula (XVII):

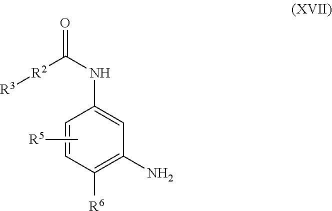

in which R², R³, R⁵, and R⁶ are as defined for general formula (I) supra, for the preparation of a compound of general formula (I) as defined supra.

In accordance with yet another aspect, the present invention covers the use of the intermediate compounds of general formula (XXII):

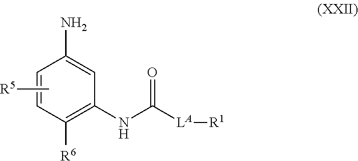

in which L^{A}, R¹, R⁵ and R⁶ are as defined for general formula (I) supra, for the preparation of a compound of general formula (I) as defined supra.

In accordance with yet another aspect, the present invention covers the use of the intermediate compounds of general formula (XXIV):

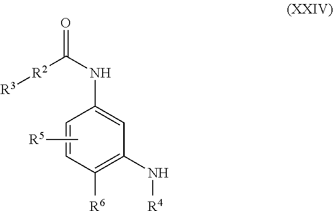

in which R², R³, R⁴, R⁵ and R⁶ are as defined for general formula (I) supra, for the preparation of a compound of general formula (I) as defined supra.

In accordance with yet another aspect, the present invention covers the use of the intermediate compounds of general formula (XXV):

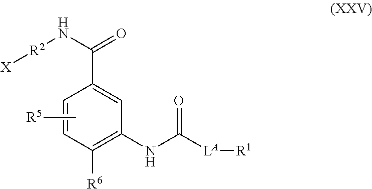

in which L^{A}, R¹, R², R⁵ and Rb are as defined for general formula (I), supra, and X represents a group enabling palladium catalysed coupling reactions, such as chloro, bromo, iodo, trifluoromethylsulfonyloxy, nonafluorobutylsulfonyloxy or a boronic acid or an ester thereof; for the preparation of a compound of general formula (I) as defined supra.

### General Synthesis of the Compounds of the Invention

The following paragraphs outline a variety of synthetic approaches suitable to prepare compounds of formulae formulae (la), (Ib), (Ic) and (Id), in which L^{A}, R¹, R², R³, R⁵ and R⁶ are as defined for the compounds of general formula (I), supra. Formulae (la) and (Ib), in which R⁴ represents hydrogen, both constitute subsets of formula (I) in that they feature different orientations of the amide linker L^{B}, which stands for —NH—C(═O)— in formula (la) whilst representing —C(═O)—NH— in formula (Ib), as shown in Scheme A. In formula (Ic), L^{B} represents —C(═O)—NH—, alike formula (Ib), and R⁴ is as defined for the compounds of general formula (I), supra, but different from hydrogen. In formula (Id), L^{B} represents —NH—C(═O)—, alike formula (la), and R⁴ is as defined for the compounds of general formula (I), supra, but different from hydrogen.

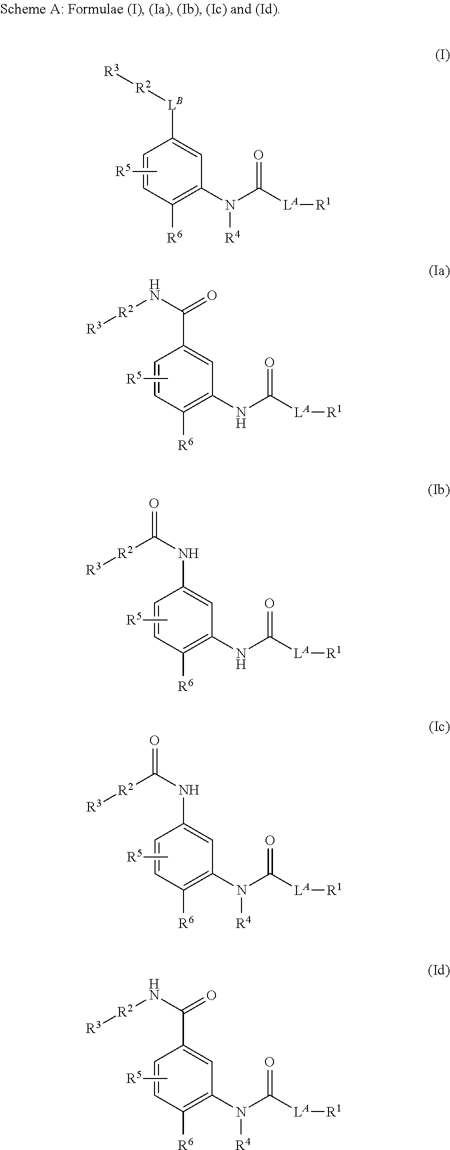

In addition to the routes described below, also other routes may be used to synthesise the target compounds, in accordance with common general knowledge of a person skilled in the art of organic synthesis. The order of transformations exemplified in the following Schemes is therefore not intended to be limiting, and suitable synthesis steps from various schemes can be combined to form additional synthesis sequences. In addition, interconversion of any of the substituents R¹, R², R³, R⁴, R⁵ and/or R⁶, can be achieved before and/or after the exemplified transformations. These modifications can be such as the introduction of protective groups, cleavage of protective groups, reduction or oxidation of functional groups, halogenation, metallation, metal catalysed coupling reactions, substitution or other reactions known to a person skilled in the art. These transformations include those which introduce a functionality allowing for further interconversion of substituents. Appropriate protective groups and their introduction and cleavage are well-known to a person skilled in the art (see for example T. W. Greene and P. G. M. Wuts in Protective Groups in Organic Synthesis, 3^{rd} edition, Wiley 1999). Specific examples are described in the subsequent paragraphs. Further, it is possible that two or more successive steps may be performed without work-up being performed between said steps, e.g. in a “one-pot” reaction, as it is well-known to a person skilled in the art.

Scheme B outlines the preparation of compounds of the formula (la), in which L^{A}, R¹, R², R³, R⁵, and R⁶ are as defined for the compounds of general formula (I), supra, starting from meta-nitrobenzoic acid derivatives (II), in which R⁵ and R⁶ are as defined for the compounds of general formula (I), supra, which can be converted into the corresponding benzoyl chlorides (Ill), by treatment with a suitable chlorinating agent, such as oxalyl chloride. Benzoic acid derivatives of the formula (II) are well known to a person skilled in the art, and are often commercially available. Said benzoyl chlorides of the formula (Ill) can be subsequently converted into amides of the general formula (V), e.g. directly by aminolysis with amines R³—R²—NH₂, in which R² and R³ are as defined for the compounds of general formula (I), supra. Alternatively, amides of the formula (V) can be accomplished in two steps by aminolysis of (Ill) or amide coupling reaction of (II) using an amine X—R²-NH₂, in which R² is as defined for the compounds of general formula (I), supra, and X stands for chloro, bromo, iodo, trifluoromethylsulfonyloxy, —O—S(═O)₂C₄F₉ (nonafluorobutylsulfonyloxy) and the like, preferably bromo, giving rise to amides of the formula (IV). Said amides can be subsequently coupled with R³-X′, in which R³ is as defined for the compounds of general formula (I), supra, and X′ stands for a hydrogen atom connected to a nitrogen atom in an aminolysis reaction to furnish amides of general formula (V). This said reaction can be performed with a base, e.g. potassium carbonate, in a solvent, e.g. N,N-dimethylformamide. Alternatively, the direct amide coupling of compounds of the formula (II) with an amino compound of the formula R³—R²—NH₂, can be accomplished for example in the presence of a tertiary aliphatic amine, such as N,N-diisopropylethylamine, and 2,4,6-tripropyl-1,3,5,2,4,6-trioxaphosphinane 2,4,6-trioxide (also known as T3P), in a suitable solvent such as N,N-dimethylformamide. The nitro group present in said amides (V) is then reduced by treatment with a suitable reducing agent, such as titanium(III)chloride, or hydrogenation in the presence of a suitable catalyst, e.g. palladium on charcoal, to give anilines of the formula (VI). Said anilines of the formula (VI) are then elaborated into compounds of the formula (la). This can be accomplished directly by reacting a compound of the formula (VI) with a carboxylic acid HO₂C-L^{A}-R¹, wherein L^{A} and R′ are as defined for the compounds of general formula (I), supra, in an amide coupling reaction, for example in the presence of a tertiary aliphatic amine, such as N,N-diisopropylethylamine, and 2,4,6-tripropyl-1,3,5,2,4,6-trioxaphosphinane 2,4,6-trioxide (also known as T3P), in a suitable solvent such as N,N-dimethylformamide. Alternatively, the transformation of anilines (VI) into compounds of the formula (la) can be performed by reaction of anilines (VI) with suitable reagents such as Cl—C(═O)-L^{A}-R¹, wherein L^{A} and R′ are as defined for compounds of the general formula (I), supra, or, in a two step synthesis firstly with Cl—C(═O)-L^{A}-LG, in which L^{A} is as defined for the compounds of general formula (I), supra, and LG stands for a leaving group, preferably chloro or bromo, to give the corresponding compounds of formula (VII), which are subsequently reacted with agents suitable for the introduction of R¹, exemplified by but not limited to cyclic secondary amines, to give compounds of the formula (la).

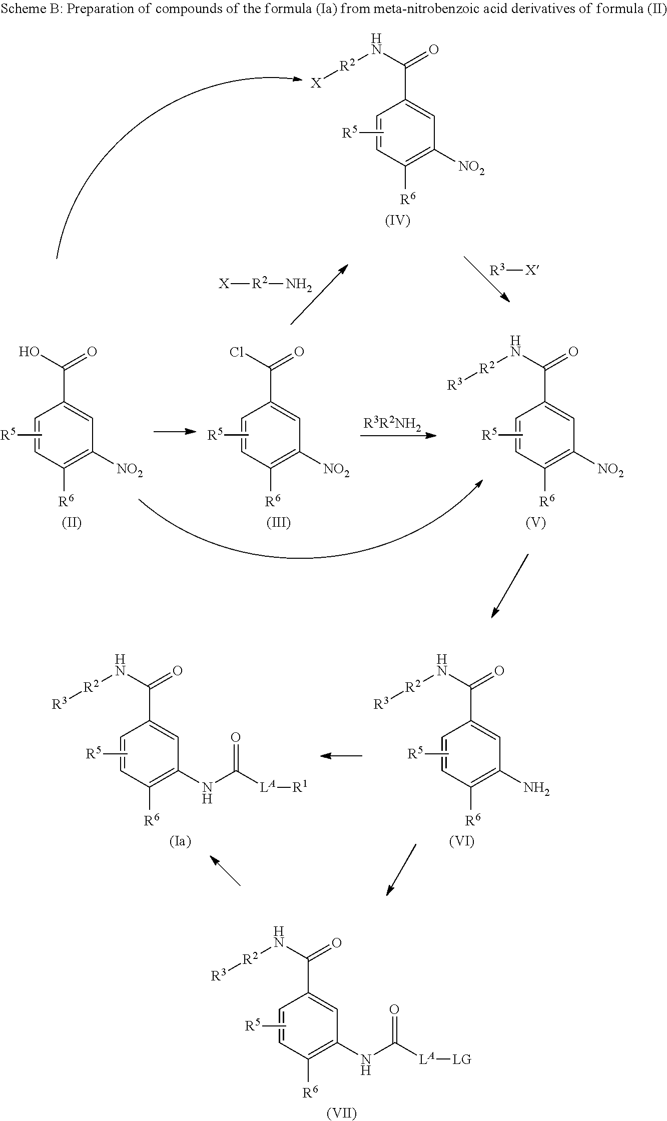

Alternatively, compounds of the formula (la) can be prepared starting from meta-aminobenzoic acid derivatives of formula (VIII), in which R⁵ and R⁶ are as defined for the compounds of general formula (I), supra, as outlined in Scheme C. Said meta-aminobenzoic acid derivatives of formula (VIII) are well known to a person skilled in the art and are commercially available in many cases. Compounds of formula (VIII) can be reacted with an amine R³R²NH₂, in which R² and R³ are as defined for the compounds of general formula (I), supra, in a standard amide coupling reaction, to give amide derivatives of formula (VI). Said compounds of formula (VI) can also be obtained by coupling the aformentioned acids of formula (VIII) with an amine X—R²—NH₂, in which R² is as defined for the compounds of general formula (I), supra, and X stands for chloro, bromo, iodo, trifluoromethylsulfonyloxy or nonafluorobutylsulfonyloxy and the like, preferably bromo, giving rise to amides of the formula (IX). These compounds (IX) are subsequently subjected to an aminolysis reaction with R³—X′, in which R³ is as defined for the compounds of general formula (I), supra, and X′ stands for a hydrogen atom connected to a nitrogen atom in order to furnish amides of general formula (VI), This said reaction can be performed with a base, e.g. potassium carbonate, in a solvent, e.g. N,N-dimethylformamide. Amides of the formula (VI) are subsequently converted into compounds of formula (la) as described supra in context with Scheme B. The compounds of formula (VI) are reacted in a standard amide coupling reaction, supra, with carboxylic acids R¹-L^{A}-CO₂H, wherein L^{A} and R¹ are as defined for compounds of the general formula (I), supra, or the corresponding carboxylic acid chlorides R¹-L^{A}-C(═O) CI, wherein L^{A} and R¹ are as defined for the compounds of formula (I), supra. Alternatively, this can be performed in a two step sequence reacting compounds of formula (VI) in a amide coupling reaction, supra, with LG-L^{A}-CO₂H or the corresponding carboxylic acid chloride LG-L^{A}-C(═O) CI, wherein L^{A} is as defined for compounds of the general formula (I), supra, and LG stands for a leaving group, preferably chloro or bromo, followed e.g. by aminolysis using reagents suitable for the introduction of R¹, wherein R¹ is as defined for compounds of the general formula (I), supra, exemplified by but not limited to suitable cyclic secondary amines, to give compounds of the formula (la).

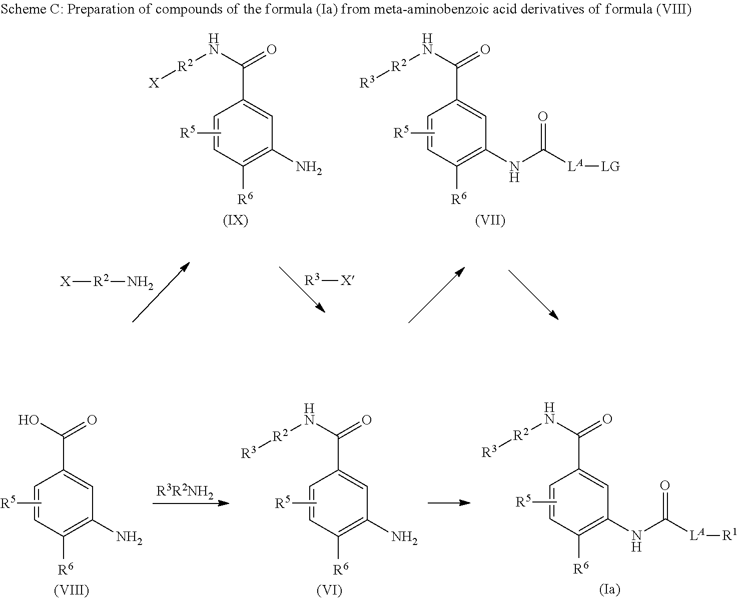

The sequence of synthetic steps can be varied as outlined in Scheme D, in order to convert meta-aminobenzoic acid derivatives of formula (VIII), in which R⁵ and R⁶ are as defined for the compounds of general formula (I), supra, into compounds of the formula (la). Said benzoic acid derivatives of the formula (VIII) can be converted into compounds of the formula (X), in which LG stands for a leaving group, preferably chloro or bromo, followed e.g. by aminolysis of compounds of the formula (X) using reagents suitable for the introduction of R¹, exemplified by but not limited to suitable cyclic secondary amines, to give compounds of the formula (XI). Additionally, compounds of the formula (XI) can be directly synthesised from amino derivatives of the formula (VIII) and carboxylic acids R¹-L^{A}-CO₂H or the corresponding carboxylic acid chloride R¹-L^{A}-C(═O) CI, wherein L^{A} and R¹ are as defined for the compounds of general formula (I), supra. Subsequently, the carboxy group present in compounds of the formula (XI) can be coupled with an amine R³R²NH₂, in which R² and R³ are as defined for the compounds of general formula (I), supra, in an amide coupling reaction, for example in the presence of a tertiary aliphatic amine, such as N,N-diisopropylethylamine, and 2,4,6-tripropyl-1,3,5,2,4,6-trioxaphosphinane 2,4,6-trioxide (also known as T3P), in a suitable solvent such as N,N-dimethylformamide, to afford compounds of the formula (la). Compounds of the formula (la) can be also synthesised from compounds of the formula (XI) in a two step sequence, reacting compounds of the formula (XI) and amines of the formula X—R²—NH₂, wherein R² is as defined for the compounds of general formual (I), supra, and X stands for chloro, bromo, iodo, trifluoromethylsulfonyloxy, nonafluorobutylsulfonyloxy and the like, preferably bromo, in an amide coupling reaction, as described supra, followed by an aminolysis with R³—X′, wherein R³ is as defined for compounds of the general formula (I), supra, and X′ stands for an hydrogen atom connected to a nitrogen atom affording compounds of the formula (la). This said reaction can be performed with a base, e.g. potassium carbonate, in a solvent, e.g. N,N-dimethylformamide.

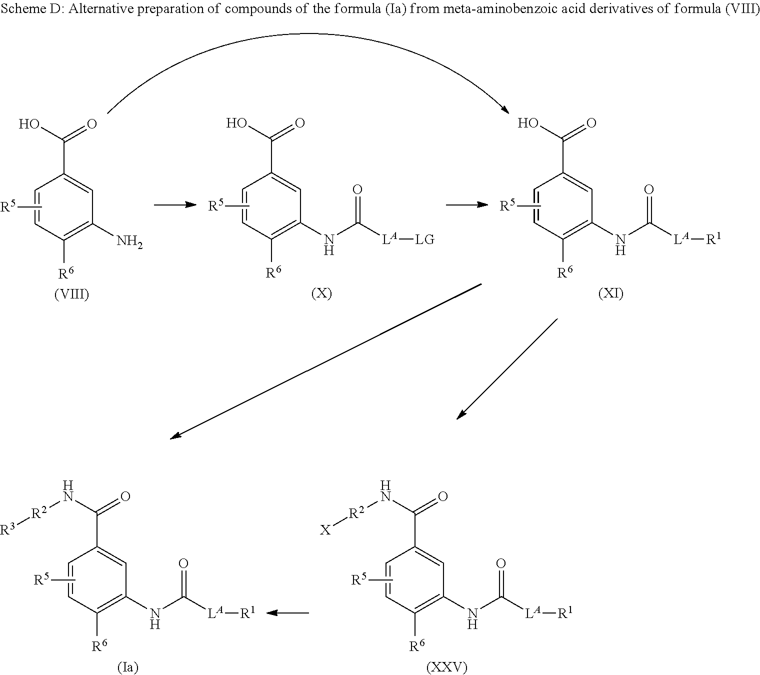

Instead of said benzoic acid derivatives of formula (VIII), also the corresponding ester analogues of formula (XII), in which R⁵ and R⁶ are as defined for the compounds of general formula (I), supra, and in which R^{E} stands for a C₁-C₆-alkyl group, preferably methyl or ethyl, can be employed in a similar fashion in order to prepare compounds of the formula (la), as outlined in Scheme E. Esters of the formula (XII) are well known to a person skilled in the art, and are commercially available in many cases. Such aminoesters of the formula (XII) can be synthesised from meta-nitrocarboxylic acids of the formula (Xlla), wherein R⁵ and R⁶ are as defined for the compounds of the general formula (I), supra, in an esterification reaction under acidic catalysis, e.g. sulphuric acid, and elevated temperature, e.g. reflux temperature of the solvent, with alcohols of the formula R^{E}—OH, in which R^{E} stands for a C₁-C₆-alkyl group, preferably methyl or ethyl, giving compounds of the formula (Xllb). The nitro group present in said esters (Xllb) is then reduced by treatment with a suitable reducing agent, such as titanium(III)chloride, or hydrogenation in the presence of a suitable catalyst, e.g. palladium on charcoal, to give anilines of the formula (XII). Elaboration of said benzoic acid esters of formula (XII) into compounds of formula (XIV), in which L^{A} and R¹ are as defined for the compounds of general formula (I), supra, can proceed via compounds of formula (XIII), in which LG stands for a leaving group, preferably chloro or bromo, and can be performed analogously as described in context with Scheme D. Subsequently, the ester group present in compounds of formula (XIV) can be saponified by reaction with e.g. lithium hydroxide to yield the lithium salt of the formula (Xla) or after acidification with acid, e.g. hydrochloric acid, the carboxylic acid of the the formula (XI). Said lithium salt of formula (Xla) or the corresponding carboxylic acid of the formula (XI) is then converted into compounds of formula (Ia) by an amide coupling reaction with R³R²NH₂, wherein R² and R³ are as defined for compounds of the general formula (I), supra, giving rise to compounds of the formula (Ia). Said compounds of formula (Ia) can be synthesised alternatively in a two step sequence via an amide coupling reaction of compounds of the formula (XI) or (Xla) with X—R²—NH₂, wherein R² is as defined for compounds of the general formula (I), supra, and X stands for chloro, bromo, iodo, trifluoromethylsulfonyloxy or nonafluorobutylsulfonyloxy and the like, preferably bromo, following an aminolysis with R³—X′, wherein R³ is as defined for compounds of the general formula (I), supra, and X′ stands for a hydrogen atom connected with a nitrogen atom, said reaction can be performed with a base, e.g. potassium carbonate, in a solvent, e.g. N,N-dimethylformamide yielding compounds of the formula (Ia).

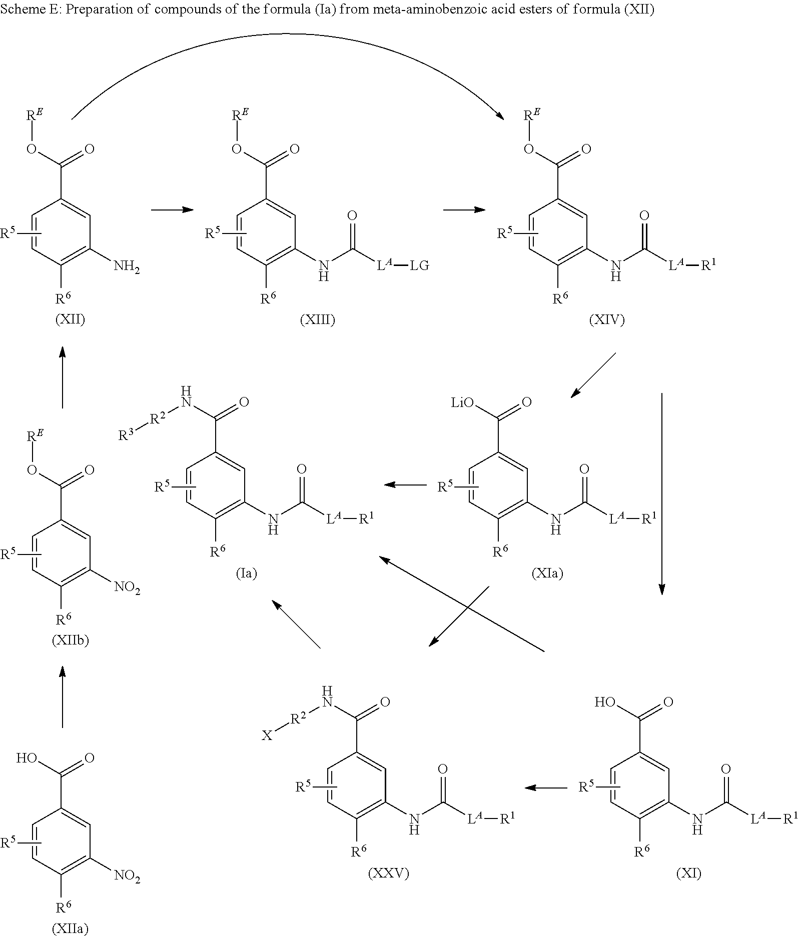

A first approach to compounds of the formula (Ib) from meta-nitroaniline derivatives of formula (XV), in which R⁵ and R⁶ are as defined for the compounds of general formula (I), supra, is outlined in Scheme F. Said meta-nitroaniline derivatives of formula (XV) are well known to a person skilled in the art, and are often commercially available. They can be converted into amide derivatives of formula (XVI) e.g. by reacting with a carboxylic acid chloride R³—R²—C(═O)Cl , in which R² and R³ are as defined for the compounds of general formula (I), supra, in the presence of a suitable base, such as potassium carbonate, and in a suitable solvent, such as acetonitrile. Basic solvents, such as pyridine, can take over both the role of a base and of a solvent, respectively. In another method the corresponding ester R³—R²—CO₂R^{E}, wherein R² and R³ are as defined for the compounds of general formual (I), supra, and R^{E} stands for a C₁-C₆-alkyl group, preferably methyl or ethyl, can be coupled with the compounds of formula (XV) under trimethylaluminium catalysis (e.g. Tetrahedron Letters 2008, 49, 5687) affording compounds of formula (XVI). Alternatively, conversion of (XV) into (XVI) can be performed via standard amide coupling reactions. In addition, nitro compounds of formula (XV) can be converted into compounds of the formula (XVI) in a two step sequence. This can be performed via amide coupling reactions, methods are described in the context with Scheme B, supra, of (XV) with X—R²—CO₂H or the corresponding ester X—R²—CO₂R^{E} , R² is as defined for the compounds of general formula (I), supra, X stands for chloro, bromo, iodo, trifluoromethylsulfonyloxy, nonafluorobutylsulfonyloxy and the like, preferably bromo, and R^{E} stands for a C₁-C₆-alkyl group, preferably methyl or ethyl, obtaining compounds of the formula (XVIa). Said compounds (XVIa) can be transformed into compounds of the formula (XVI) via aminolysis with R³—X′, wherein R³ is as defined for the compounds of general formula (I), supra, and X′ stands for a hydrogen atom connected with a nitrogen atom, yielding the compounds of the formula (XVI). Said reaction can be performed with a base, e.g. potassium carbonate, in a solvent, e.g. N,N-dimethylformamide. The nitro group present in amides of the formula (XVI) can be subsequently reduced e.g. by hydrogenation in the presence of a suitable catalyst, e.g. palladium on charcoal, to give the corresponding aniline derivatives of formula (XVII). Said anilines of the formula (XVII) can then be elaborated into compounds of the formula (Ib). This can be accomplished directly by reacting a compound of the formula (XVII) with a carboxylic acid HO₂C-L^{A}-R¹, wherein L^{A} and R¹ are as defined for the compounds of general formula (I), supra, in an amide coupling reaction, for example in the presence of a tertiary aliphatic amine, such as N,N-diisopropylethylamine, and 2,4,6-tripropyl-1,3,5,2,4,6-trioxaphosphinane 2,4,6-trioxide (also known as T3P), in a suitable solvent such as N,N-dimethylformamide. Alternatively, the transformation of anilines (XVII) into compounds of the formula (Ib) can be performed by reaction of anilines (XVII) with suitable reagents, such as Cl—C(═O)-L^{A}-LG, in which L^{A} is as defined for the compounds of general formula (I), and LG stands for a leaving group, preferably chloro or bromo, to give the corresponding compounds of formula (XVIII), which are subsequently reacted with agents suitable for the introduction of R¹, wherein R¹ is as defined for compounds of the formula (I), supra, exemplified by but not limited to cyclic secondary amines, to give compounds of the formula (Ib).

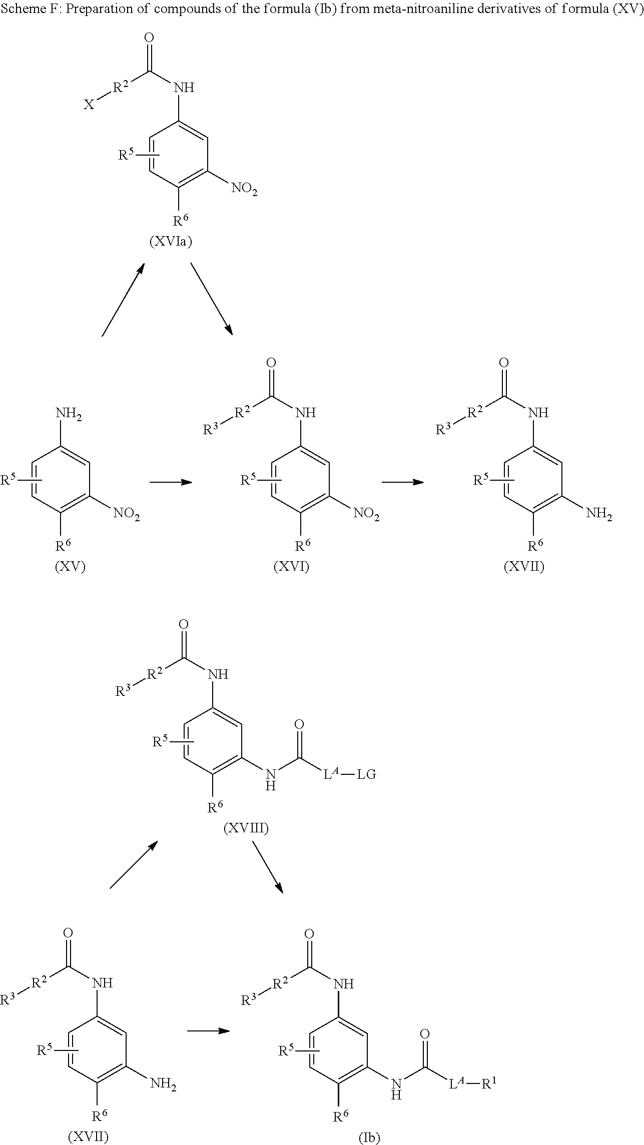

Scheme G outlines an approach complimentary to Scheme F as an alternative synthesis route for compounds of the formula (Ib), from meta-nitroaniline derivatives of formula (XIX), in which R⁵ and R⁶ are as defined for the compounds of general formula (I), supra, and which differ from the compounds of formula (XV) by the inverse arrangement of their nitro and amino groups, respectively. Said meta-nitroaniline derivatives of formula (XIX) are well known to a person skilled in the art, and are often commercially available. They can be converted into amide derivatives of formula (XX), in which L^{A} is as defined for the compounds of general formula (I), supra, and in which LG stands for a leaving group, preferably chloro or bromo, by reacting with a carboxylic acid LG-L^{A}-CO₂H or the corresponding carboxylic acid chloride LG-L^{A}-C(═O)CI, in which L^{A} is as defined for compounds of the general formula (I), supra, in a standard amide coupling reaction. Said amides of the formula (XX) can be subsequently converted into compounds of the formula (XXI), in which R¹ is as defined for the compounds of general formula (I), supra, using reagents suitable for the introduction of R¹, exemplified by but not limited to cyclic secondary amines. Alternatively, converting compounds (XIX) into compounds of formula (XXI) can be accomplished directly by reacting compounds of the formula R¹-L^{A}-COOH or the corresponding carboxylic acid chloride R¹-L^{A}-C(═O)Cl, wherein R¹ and L^{A} are as defined for the compounds of general formula (I), supra, in an amide coupling reaction, supra. The nitro group present in amides of the formula (XXI) is then reduced e.g. by hydrogenation in the presence of a suitable catalyst, e.g. palladium on charcoal, to give the corresponding aniline derivatives of formula (XXII). Compounds of formula (XXII) can be reacted with a carboxylic acid R³R²CO₂H, wherein R² and R³ are as defined for the compounds of general formula (I), supra, in an amide coupling reaction, for example in the presence of a tertiary aliphatic amine, such as N,N-diisopropylethylamine, and 2,4,6-tripropyl-1,3,5,2,4,6-trioxaphosphinane 2,4,6-trioxide (also known as T3P), in a suitable solvent such as N,N-dimethylformamide, to give compounds of the formula (Ib). The corresponding ester R³R²OR²CO₂R^{E}, wherein R² and R³ are as defined for the compounds of general formula (I), supra, and R^{E} stands for a C₁-C₆-alkyl group, preferably methyl or ethyl, can be coupled with the compounds of formula (XXII) under trimethylaluminium catalysis as described in the context with Scheme F. The compounds of formula (Ib) can also be obtained by coupling the aformentioned anilines of formula (XXII) with a carboxylic acid X—R²-CO₂H or the corresponding ester X—R²-CO₂R^{E}, in which R² is as defined for the compounds of general formula (I), supra, X stands for chloro, bromo, iodo, trifluoromethylsulfonyloxy or nonafluorobutylsulfonyloxy and the like, preferably bromo, and R^{E} stands for a C₁-C₆-alkyl group, preferably methyl or ethyl, giving rise to amides of the formula (XXIII). These can be subsequently subjected to an aminolysis with R³—X′, wherein R³ is as defined for the compounds of general formula (I), supra, and X′ stands for a hydrogen atom connected with a nitrogen atom. Said reaction can be performed with a base, e.g. potassium carbonate, in a solvent, e.g. N,N-dimethylformamide.

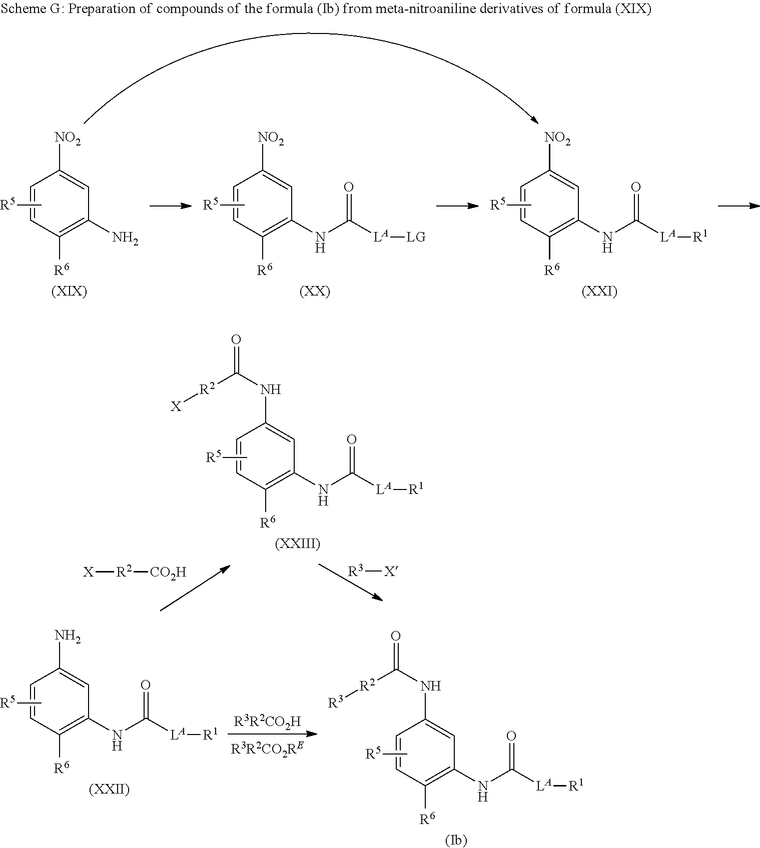

Instead of benzoic acid ester derivatives of formula (XII), as depicted in Scheme E, also the corresponding meta-substituted analogues of formula (XXVI), in which R⁵ and R⁶ are as defined for the compounds of general formula (I), and in which A stands for a chloro, bromo, iodo, trifluoromethylsulfonyloxy, nonafluorobutylsulfonyloxy and the like, preferably bromo, can be employed in a similar fashion in order to prepare compounds of the formula (Xla), as outlined in Scheme H. Compounds of the formula (XXVI) are well known to a person skilled in the art, and are commercially available in many cases. Elaboration of said compounds of formula (XXVI) into compounds of formula (XXVIII), in which L^{A} and R¹ are as defined for the compounds of general formula (I), supra, can proceed via compounds of formula (XXVII), in which LG stands for a leaving group, preferably chloro or bromo, and can be performed analogously as described in context with Scheme D. Alternatively, conversion of (XXVI) into (XXVIII) can be performed via standard amide coupling reactions, as described supra, of carboxylic acids of the formula R¹-L^{A}-COOH, R¹ and L^{A} are as defined for the general formula (I), supra. The compounds of formula (XXVIII) are transformed into the corresponding esters of the formula (XIV), wherein R^{E} stands for a C₁-C₆-alkyl, preferably methyl or ethyl. This kind of reaction can be performed under palladium catalysis, for example dichloropalladium-propane-1,3-diylbis(diphenylphosphine), in an alcohol R^{E}-OH, R^{E} is as defined as supra, e.g. ethanol, with an aliphatic amine, e.g. triethylamine, at elevated temperatures ranging from 50-150° C., e.g. 100° C., and with pressurised carbon monoxide, e.g. 10-20 bar, affording compounds of the formula (XIV). Subsequently, the ester group present in compounds of formula (XIV) can be saponified by reaction with e.g. lithium hydroxide to yield the lithium salt of the formula (Xla).

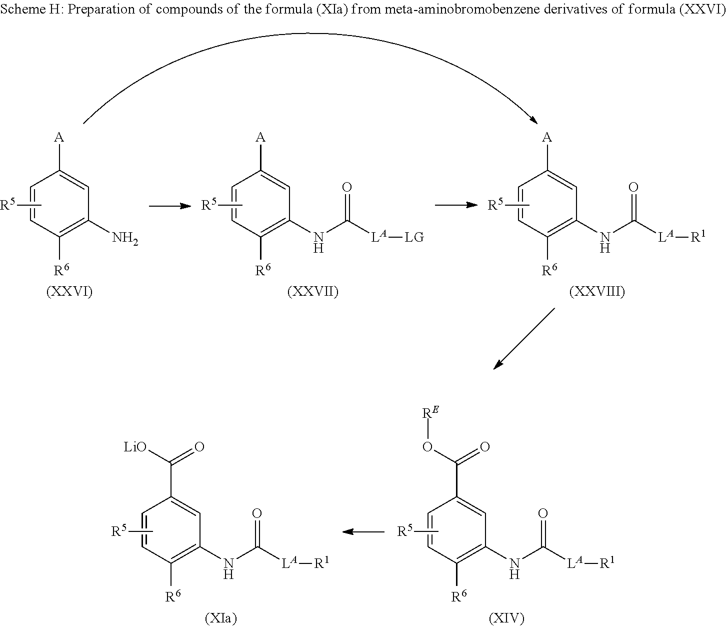

Scheme I illustrates the introduction of R⁴ groups different from hydrogen. In order to do so, primary anilines of the formula (XVII), in which R², R³, R⁵, and R⁶ are as defined for the compounds of general formula (I), supra, and which can be prepared for example according to Scheme F, can be converted into secondary anilines of the formula (XXIX), in which R⁴ is as defined for the compounds of general formula (I), supra, but different from hydrogen. This can be accomplished by various methods known to a person skilled in the art, such as a reductive amination with an aldehyde suitable to confer R⁴, e.g. benzaldehyde for R⁴=benzyl, in the presence of a suitable borohydride reagent, such as sodium triacetoxyborohydride, and in the presence of a suitable acid, such as acetic acid, in a suitable solvent, such as a chlorinated hydrocarbon, preferably dichloromethane. The resulting compounds of the formula (XXIX) are subsequently elaborated into compounds of the formula (Ic), in which L^{A}, R¹, R², R³, R⁴, R⁵ and R⁶ are as defined for the compounds of general formula (I), supra, with the proviso that R⁴ is different from hydrogen.

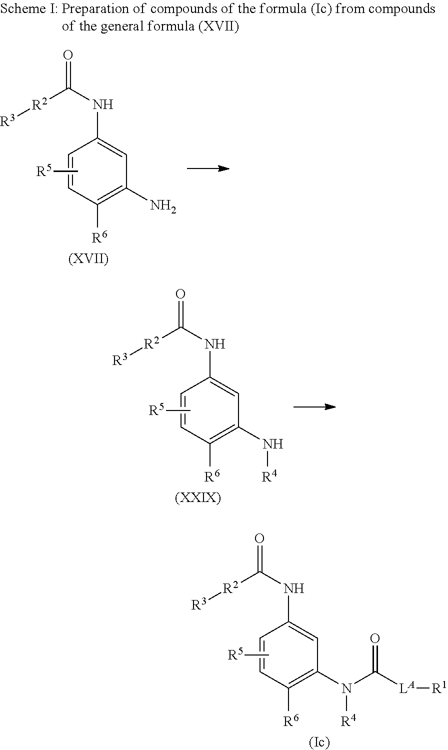

Scheme J illustrates the introduction of R⁴ groups different from hydrogen. In order to do so, primary anilines of the formula (VI), in which R², R³, R⁵, and R⁶ are as defined for the compounds of general formula (I), supra, and which can be prepared for example according to Scheme C, can be converted into secondary anilines of the formula (XXX), in which R⁴ is as defined for the compounds of general formula (I), supra, but different from hydrogen. This can be accomplished by various methods known to a person skilled in the art, such as a reductive amination with an aldehyde suitable to confer R⁴, e.g. benzaldehyde for R⁴=benzyl, in the presence of a suitable borohydride reagent, such as sodium triacetoxyborohydride, and in the presence of a suitable acid, such as acetic acid, in a suitable solvent, such as a chlorinated hydrocarbon, preferably dichloromethane. The resulting compounds of the formula (XXX) are subsequently elaborated into compounds of the formula (Id), in which L^{A}, Fe, R², R³, R⁴, R⁵ and R⁶ are as defined for the compounds of general formula (I), supra, with the proviso that R⁴ is different from hydrogen.

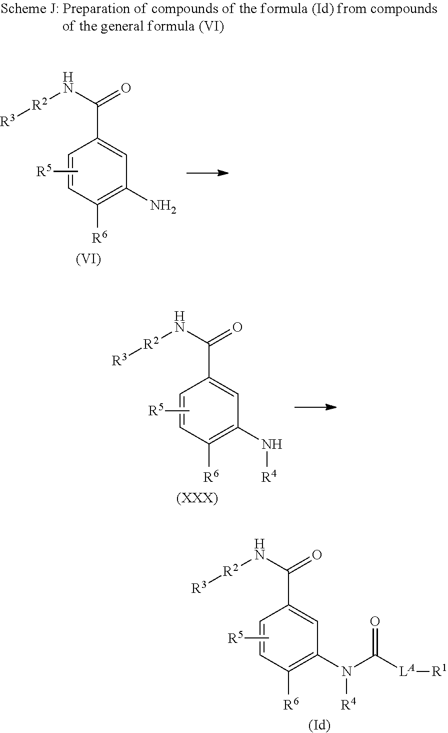

Compunds of the formula (XXXII), wherein R³ is as defined for the compounds of general formual (I), supra, can be prepared by reacting them with compounds of the formula R³-X′, wherein R³ is as defined for the compounds of general formula (I), supra, and X′ stands for a hydrogen atom connected with a nitrogen atom, under basic catalysis, e.g. potassium carbonate, in a solvent, e.g. N,N-dimethylformamide, yielding compounds of the formula (XXXII).

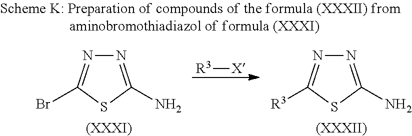

Compunds of the formula (XXXIV), wherein R³ is as defined for the compounds of general formual (I), supra, can be prepared by reacting them with compounds of the formula R³-X′, wherein R³ is as defined for the compounds of general formula (I), supra, and X′ stands for a hydrogen atom connected with a nitrogen atom, under basic catalysis, e.g. potassium carbonate, in a solvent, e.g. N,N-dimethylformamide yielding compounds of the formula (XXXIV).

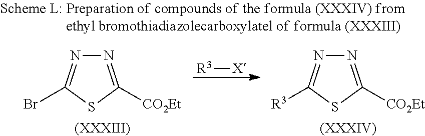

Further details (reaction conditions, suitable solvents etc.) can be obtained from the experimental section below.

In the present text, in particular in the Experimental Section, for the synthesis of intermediates and of examples of the present invention, when a compound is mentioned as a salt form with the corresponding base or acid, the exact stoichiometric composition of said salt form, as obtained by the respective preparation and/or purification process, is, in most cases, unknown.

Unless specified otherwise, suffixes to chemical names or structural formulae such as “hydrochloride”, “trifluoroacetate”, “sodium salt”, or “x HCI”, “x CF₃COOH”, “x Na⁺”, for example, are to be understood as not a stoichiometric specification, but solely as a salt form.

This applies analogously to cases in which synthesis intermediates or example compounds or salts thereof have been obtained, by the preparation and/or purification processes described, as solvates, such as hydrates with (if defined) unknown stoichiometric composition.

Experimental Section

The following table lists the abbreviations used in this paragraph, and in the examples section.

| Abbreviation | Meaning |
|---|---|
| anh | anhydrous |
| br. | broad signal (in NMR data) |
| d | day(s) |
| DAD | Diode Array Detector |
| DCM | dichloromethane |
| DME | 1,2-dimethoxyethane |
| DMF | N,N-dimethylformamide |
| DMSO | dimethyl sulfoxide |
| ELSD | Evaporative Light Scattering Detector |
| ESI | electrospray ionisation |
| EtOAc | ethyl acetate |
| h | hour |
| HPLC, LC | high performance liquid chromatography |
| m/z | mass-to-charge ratio (in mass spectrum) |
| mc | multiplet centred |
| MeOH | methanol |
| min | Minute |
| MPLC | medium pressure liquid chromatography |
| MS | mass spectroscopy |
| neg | negative |
| NMR | nuclear magnetic resonance |
| PE | petroleum ether |
| pos | positive |
| ppm | Chemical shift δ in parts per million |
| PYBOP | (1H-benzotriazol-1-yloxy)(tripyrrolidin-1-yl)phosphonium |
| | hexafluorophosphate |
| Rₜ | retention time |
| rt | room temperature |
| THF | tetrahydrofuran |
| TLC | thin layer chromatography |

Methods

Method 1:

Instrument: Waters Acquity UPLC-MS SQD; column: Acquity UPLC BEH C18 1.7 50×2.1 mm; Eluent A: water+0.05% vol. formic acid (98%), Eluent B: acetonitrile+0.05% vol. formic acid (98%); gradient: 0-1.6 min 1-99% B, 1.6-2.0 min 99% B; rate 0.8 mL/min; temperature: 60° C.; DAD scan: 210-400 nm; ELSD.

Method 2:

Instrument: aters Autopurificationsystem SOD; column: Waters XBrigde C18 5μ 100×30 mm; water+0.1% vol. formic acid (99%)/acetonitrile gradient; temperature: room temperature; injection: 2500 μL; DAD scan: 210-400 nm.

Method 3:

Instrument: Waters Acquity UPLC-MS SQD; column: Acquity UPLC BEH C18 1.7 50×2.1 mm; Eluent A: water+0.2% vol. ammonia (32%), Eluent B: acetonitrile; gradient: 0-1.6 min 1-99% B, 1.6-2.0 min 99% B; rate 0.8 mL/min; temperature: 60° C.; DAD scan: 210-400 nm; ELSD.

Method 4:

Instrument: Waters Acquity UPLC-MS SQD; column: Acquity UPLC BEH C18 1.7 50×2.1 mm; Eluent A: water+0.1% vol. formic acid (99%), Eluent B: acetonitrile; gradient: 0-1.6 min 1-99% B, 1.6-2.0 min 99% B; rate 0.8 mL/min; temperature: 60° C.; DAD scan: 210-400 nm; ELSD.

Method 5:

Instrument: Waters Autopurificationsystem SOD; column: Waters XBrigde C18 5μ 100×30 mm; water+0.2% vol. ammonia (32%)/acetonitrile gradient; temperature: room temperature; injection: 2500 μL; DAD scan: 210-400 nm.

Method 6:

Instrument: JASCO P2000 Polarimeter; wavelength 589 nm; temperature: 20° C.; integration time 10 s; path length 100 mm.

Method 7:

Instrument: Acquity UPLC from Waters; mass detector: LCT from Micromass (now Waters); column: Kinetex C18 from Phenomenex, 50×2.1 mm, 2.6 μm particle, 60° C.; solvent: A: water+0.05% formic acid; B: acetonitrile+0.05% formic acid; injection: 0.5 μL; rate: 1.3 mL/min; gradient 99% A, 1% B until 1.9 min linear to 1% A, 99% B; 1.9-2.10 min unchanged; until 2.20 min back to 99% A, 1% B.

The ¹H-NMR data of selected examples are listed in the form of ¹H-NMR peaklists. For each signal peak the δ value in ppm is given, followed by the signal intensity, reported in round brackets. The δ value-signal intensity pairs from different peaks are separated by commas. Therefore, a peaklist is described by the general form: δ₁ (intensity,), δ₁ (intensity2), . . . , δᵢ (intensityᵢ), . . . , δₙ (intensityₙ.

The intensity of a sharp signal correlates with the height (in cm) of the signal in a printed NMR spectrum. When compared with other signals, this data can be correlated to the real ratios of the signal intensities. In the case of broad signals, more than one peak, or the center of the signal along with their relative intensity, compared to the most intense signal displayed in the spectrum, are shown. A ¹H-NMR peaklist is similar to a classical ¹H-NMR readout, and thus usually contains all the peaks listed in a classical NMR interpretation. Moreover, similar to classical ¹H-NMR printouts, peaklists can show solvent signals, signals derived from stereoisomers of target compounds (also the subject of the invention), and/or peaks of impurities. The peaks of stereoisomers, and/or peaks of impurities are typically displayed with a lower intensity compared to the peaks of the target compounds (e.g., with a purity of >90%). Such stereoisomers and/or impurities may be typical for the particular manufacturing process, and therefore their peaks may help to identify the reproduction of our manufacturing process on the basis of “by-product fingerprints”. An expert who calculates the peaks of the target compounds by known methods (MestReC, ACD simulation, or by use of empirically evaluated expectation values), can isolate the peaks of target compounds as required, optionally using additional intensity filters. Such an operation would be similar to peak-picking in classical ¹H-NMR interpretation. A detailed description of the reporting of NMR data in the form of peaklists can be found in the publication “Citation of NMR Peaklist Data within Patent Applications” (cf. Research Disclosure Database Number 605005, 2014, 1 Aug. 2014, or http://www.researchdisclosure.com/searching-disclosures). In the peak picking routine, as described in the Research Disclosure Database Number 605005, the parameter “MinimumHeight” can be adjusted between 1% and 4%. Depending on the chemical structure and/or depending on the concentration of the measured compound it may be reasonable to set the parameter “MinimumHeight” <1%.

Intermediates

Intermediate 1

3-[(chloroacetyl)amino]-4-(trifluoromethoxy)benzoic acid

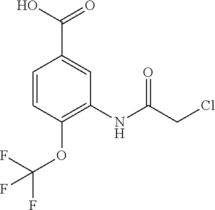 [SMILES: O=C(CCl)Nc1cc(C(=O)O)ccc1OC(F)(F)F]

To a solution of 3-amino-4-(trifluoromethoxy)benzoic acid (2.50 g, 11.3 mmol, known from WO2008/75064A1) and pyridine (1.92 mL, 23.7 mmol, 2.1 equiv) in DCM (50 mL) at 0° C. was added chloroacetyl chloride (0.95 mL, 11.9 mmol, 1.05 equiv) dropwise. The resulting mixture was allowed to warm to room temperature and was stirred at that temperature for 5 h. The resulting solution was treated with a DCM/isopropanol mixture (4:1, 50 mL). The resulting solution was washed with an aqueous 1N HCI solution (50 mL), dried (MgSO₄ anh), and concentrated under reduced pressure to give impure 3-[(chloroacetyl)amino]-4-(trifluoromethyl)benzoic acid (3.52 g). This material was used in subsequent reactions without further purification.

¹H-NMR (300 MHz, DMSO-d₆): δ [ppm]=4.35 (s, 2H), 7.52 (ddm, J=1.5, 8.7 Hz, 1H), 7.80 (dd, J=2.1, 8.7 Hz, 1H), 8.47 (d, J=2.1 Hz, 1H), 10.17 (s, 1H), 13.28 (br. s, 1H).

LC-MS (Method 3): Rₜ=0.95 min; MS (ESlpos): m/z=298 ([M+H]⁺, 100%); MS (ESlneg): m/z=296 ([M−H]⁻, 100%), 593 ([2M−H]⁻, 100%).

Intermediate 2

3-{[(4-methylpiperazin-1-yl)pacetyl]amino}-4-(trifluoromethoxy)benzoic acid hydrochloride (1:1)

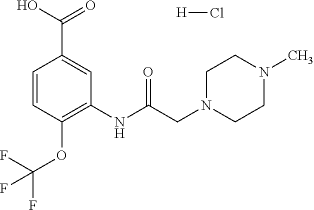 [SMILES: CN1CCN(CC(=O)Nc2cc(C(=O)O)ccc2OC(F)(F)F)CC1 ; Cl]

To a solution of intermediate 1 (1.50 g, 5.04 mmol) in DMF (45 mL) was added triethylamine (1.05 mL, 7.56 mmol), potassium iodide (126 mg, 0.76 mmol) and 1-methylpiperazine (0.84 mL, 7.56 mmol). The reaction mixture was stirred over night at room temperature. The mixture was concentrated. The remaining residue was triturated with water, and a 1M aqueous solution of hydrogen chloride was added until a pH of 4 was achieved. The mixture was saturated with sodium chloride and extracted three times with a mixture of DCM/isopropanol 4:1. The combined organic phases were dried over sodium sulfate and concentrated to yield the desired crude material (1.62 g, 69% of theory), which was used in the next step without further purification.

¹H-NMR (300 MHz, DMSO-d₆) δ [ppm]=2.60 (s, 3H), 2.70-2.85 (m, 4H), 2.90-3.03 (m, 4H), 3.31 (s, 2H), 7.50-7.60 (m, 1H), 7.81 (dd, 1H), 8.67 (d, 1H), 9.83 (s, 1H).

LC-MS (Method 4): Rₜ=0.58 min; MS (ESlpos): m/z=362 [M−HCI+H]⁺.

Intermediate 3

N-[5-bromo-2-(trifluoromethoxy)phenyl]-2-chloroacetamide

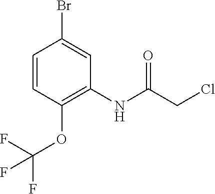 [SMILES: O=C(CCl)Nc1cc(Br)ccc1OC(F)(F)F]

240 g (0.937 mol) of 5-bromo-2-(trifluoromethoxy)aniline were dissolved in 2400 mL of anh toluene. 112 mL (1.406 mol) of chloroacetyl chloride were added. It was stirred for 2 h at 100° C. The reaction mixture was concentrated under vacuum. The residue was treated with 600 mL of cyclopentyl methyl ether and concentrated again. This procedure was performed twice yielding 324 g of the title compound.

¹H-NMR (400MHz, DMSO-d₆): δ [ppm]=4.39 (s, 2H), 7.40-7.44 (m, 1H), 7.49 (dd, 1H), 8.20 (d, 1H), 10.23 (s, 1H).

LC-MS (Method 4): Rₜ=1.27 min; MS (ESlpos): m/z=332 [M+H]⁺.

Intermediate 4

N-[5-bromo-2-(trifluoromethoxy)phenyl]-2-(4-methylpiperazin-1-yl)pacetamide

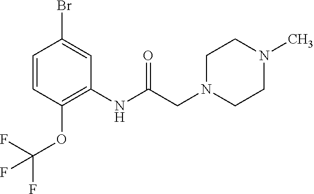 [SMILES: CN1CCN(CC(=O)Nc2cc(Br)ccc2OC(F)(F)F)CC1]

162 g (0.487 mol) of N-[5-bromo-2-(trifluoromethoxy)phenyl]-2-chloroacetamide (intermediate 3) were dissolved in 1620 mL of anh DMF. 136 mL (0.974 mol) of N,N-diethylethanamine and 16.2 g (97.44 mmol) of potassium iodide were added. It was stirred over night at rt. A second batch of the same size was prepared under analogous conditions. The two batches were combined. The reaction mixtures were concentrated and the residue was stirred with 3 L of water and 700 mL of ethanol for 1 h. The solid was filtered off under suction and dried at 50° C. under vacuum to afford 317 g (82% of theory) of the title compound.

¹H-NMR (400MHz, DMSO-d₆): δ [ppm]=2.18 (s, 3H), 2.21-2.48 (m, 4H), 2.52-2.64 (m, 4H), 3.19 (s, 2H), 7.39-7.47 (m, 2H), 8.54 (d, 1H), 9.92 (s, 1H).

LC-MS (Method 1): Rₜ=0.81 min; MS (ESlpos): m/z=396 [M+H]⁺.

Intermediate 5

ethyl 3-{[(4-methylpiperazin-1-yl )pacetyl]amino}-4-(trifluoromethoxy)benzoate

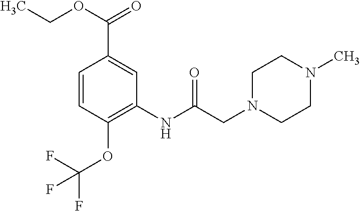 [SMILES: CCOC(=O)c1ccc(OC(F)(F)F)c(NC(=O)CN2CCN(C)CC2)c1]

60 g (0.151 mol) of N-[5-bromo-2-(trifluoromethoxy)phenyl]-2-(4-methylpiperazin-1-yl)pacetamide (intermediate 4) were dissolved in 600 mL of ethanol. 450 mg (0.76 mmol) of dichloropalladium—propane-1,3-diyIbis(diphenylphosphine) (1:1) and 53 mL (0.380 mol) of N,N-diethylethanamine were added. The 2000 mL autoclave was charged with 12.5 bar of carbon monoxide and was stirred for 16 h at 100° C. The reaction mixture was concentrated under vacuum and the residue was treated with dichloromethane. The insoluble material was filtered off and washed with dichloromethane. The filtrate was concentrated under vacuum and purified on silica gel (gradient dichloromethane/methanol) to yield 54 g (92% of theory) of the title compound, which contained approximately 0.5 mole of N,N-diethylethanamine. ¹H-NMR (400 MHz, DMSO-d₆): δ [ppm]=1.31 (t, 3H), 2.24 (s, 3H), 2.37-2.53 (m, 4H), 2.60 (br. s, 4H), 3.20 (s, 2H), 4.32 (q, 2H), 7.55-7.60 (m, 1H), 7.78 (dd, 1H), 8.86 (d, 1H), 9.89 (s, 1H).

LC-MS (Method 4): Rₜ=0.81 min; MS (ESIpos): m/z=390 [M+H]⁺.

Intermediate 6

lithium 3-{[(4-methylpiperazin-1-yl)pacetyl]amino}-4-(trifluoromethoxy)benzoate

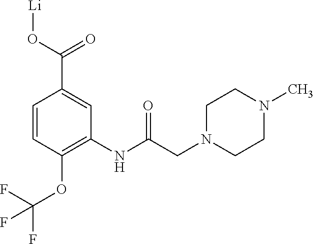

20 g (51.36 mmol) of ethyl 3-{[(4-methylpiperazin-1-yl)pacetyl]amino}-4-(trifluoromethoxy)benzoate (intermediate 5) were dissolved in 50 mL of dioxane and 2 mL of water. 3.23 g (77.05 mmol) of lithium hydroxide monohydrate were added and it was stirred for 24 h at rt. The precipitate was filtered off and washed with dioxane to yield 17.0 g (90% of theory) of the title compound, which was used without further treatment.

¹H-NMR (300MHz, DMSO-d₆): δ [ppm]=2.15 (s, 3H), 2.36 (br. s, 4H), 2.54 (br. s, 4H), 3.13 (s, 2H), 7.28 (dd, 1H), 7.67 (dd, 1H), 8.70 (s, 1H), 9.70 (br. s, 1H).

LC-MS (Method 1): Rₜ=0.61 min; MS (ESlpos): m/z=362 [M+2H−Li]⁺.

Intermediate 7

3-[(morpholin-4-ylacetyl)amino]-4-(trifluoromethoxy)benzoic acid

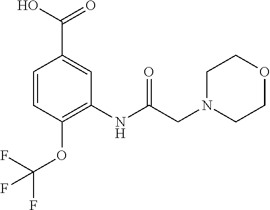 [SMILES: O=C(CN1CCOCC1)Nc1cc(C(=O)O)ccc1OC(F)(F)F]

To a solution of the compound of intermediate 1 (13.5 g, 45.2 mmol) in DMF (200 mL) was added morpholine (7.9 mL, 90.5 mmol, 2.0 equiv), triethylamine (12.6 mL, 90.5 mmol, 2.0 equiv) and potassium iodide (1.50 g, 9.05 mmol, 0.2 equiv). The reaction mixture was stirred at room temperature for 2 days. The resulting mixture was concentrated, the remaining material was treated with water and extracted with a DCM/isopropanol solution (4:1). The combined organic phases were washed with saturated brine, dried (Na₂SO₄ anh), and concentrated under reduced pressure to give 15.9 g (91% of theory) of the title compound.

LC-MS (Method 4): Rₜ=0.74 min; MS (ESlpos): m/z=349 [M+H]⁺.

Intermediate 8

5-(piperidin-1-yl)-1,3,4-thiadiazol-2-amine

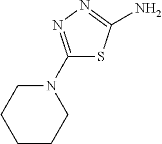 [SMILES: Nc1nnc(N2CCCCC2)s1]

1.00 g (5.39 mmol, 1.0 equiv.) of 5-bromo-1,3,4-thiadiazol-2-amine was provided in 12 mL of DMF, 0.69 mL (7.00 mmol, 1.3 equiv.) of piperidine and 1.49 g (10.78 mmol, 2.0 equiv.) of potassium carbonate were added, and the mixture was stirred at 80° C. over night. After filtration, the remaining solution was concentrated, 895 mg (87% of theory) of the title compound were obtained, which were used without further purification.

¹H-NMR (300 MHz, DMSO-d₆) δ [ppm]: 1.536 (10.21), 1.547 (16.00), 1.562 (6.55), 1.572 (3.54), 2.729 (1.50), 2.888 (1.45), 3.192 (5.41), 3.324 (15.26), 6.404 (8.34).

LC-MS (Method 4): Rₜ=0.54 min; MS (ESlpos): m/z=185 [M+H]⁺.

Intermediate 9

tert-butyl 4-(5-amino-1,3,4-thiadiazol-2-yl)piperazine-1-carboxylate

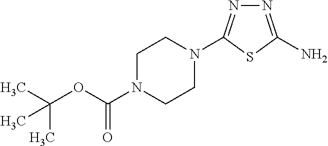 [SMILES: CC(C)(C)OC(=O)N1CCN(c2nnc(N)s2)CC1]

1.00 g (5.39 mmol, 1.0 equiv.) of 5-bromo-1,3,4-thiadiazol-2-amine was provided in 12 mL of DMF, 0.78 mL (7.00 mmol, 1.3 equiv.) of tert-butyl piperazine-1-carboxylate and 1.49 g (10.8 mmol, 2.0 equiv.) of potassium carbonate were added, and the mixture was stirred at 80° C. over night. After filtration, the remaining solution was concentrated, 2.10 g of the title compound were obtained, which were used without further purification.

¹H-NMR (400 MHz, DMSO-d₆) δ [ppm]: 1.384 (4.67), 1.408 (16.00), 2.729 (11.73), 2.889 (14.90), 2.900 (0.42), 3.182 (1.04), 3.195 (1.65), 3.200 (1.29), 3.208 (1.63), 3.395 (1.02), 3.403 (0.98), 3.409 (1.26), 3.421 (0.87), 6.519 (1.65), 7.950 (1.88).

LC-MS (Method 4): Rₜ=0.77 min; MS (ESlpos): m/z=286 [M+H]⁺.

Intermediate 10

methyl 4-(5-amino-1,3,4-thiadiazol-2-yl)piperazine-1-carboxylate

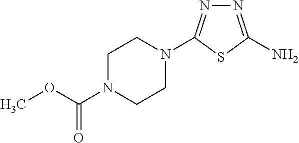 [SMILES: COC(=O)N1CCN(c2nnc(N)s2)CC1]

1.00 g (5.39 mmol, 1.0 equiv.) of 5-bromo-1,3,4-thiadiazol-2-amine was provided in 12 mL of DMF, 0.78 mL (7.00 mmol, 1.3 equiv.) of methyl piperazine-1-carboxylate and 1.49 g (10.8 mmol, 2.0 equiv.) of potassium carbonate were added, and the mixture was stirred at 80° C. over night. After filtration, the remaining solution was concentrated, 0.80 g (55% of theory) of the title compound were obtained, which were used without further purification.

¹H-NMR (400 MHz, DMSO-d₆) δ [ppm]: 2.729 (1.16), 2.889 (1.44), 3.207 (2.68), 3.219 (3.67), 3.224 (2.62), 3.233 (3.30), 3.349 (0.49), 3.444 (2.94), 3.452 (2.45), 3.458 (3.45), 3.470 (2.37), 3.580 (0.46), 3.604 (0.74), 3.615 (16.00), 6.523 (3.89).

LC-MS (Method 3): Rₜ=0.58 min; MS (ESlpos): m/z=244 [M+H]⁺.

Intermediate 11

5-(4-methylpiperidin-1-yl)-1,3,4-thiadiazol-2-amine

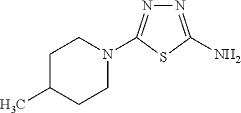

1.00 g (5.55 mmol, 1.0 equiv.) of 5-bromo-1,3,4-thiadiazol-2-amine was provided in 12 mL of DMF, 716 mg (7.22 mmol, 1.3 equiv.) of 4-methylpiperidine and 1.54 g (11.1 mmol, 2.0 equiv.) of potassium carbonate were added, and the mixture was stirred at 80° C. over night. After filtration, the remaining solution was concentrated, 1.20 g of the title compound were obtained, which were used without further purification.

¹H-NMR (400 MHz, DMSO-d₆) δ [ppm]: 0.854 (0.42), 0.871 (0.52), 0.878 (0.51), 0.902 (15.81), 0.918 (16.00), 1.116 (0.88), 1.126 (0.93), 1.144 (1.96), 1.146 (2.15), 1.156 (2.28), 1.175 (2.54), 1.186 (2.56), 1.207 (1.26), 1.218 (1.20), 1.479 (0.43), 1.488 (0.70), 1.497 (0.76), 1.505 (0.95), 1.516 (1.24), 1.525 (1.09), 1.533 (1.15), 1.542 (1.01), 1.553 (0.68), 1.559 (0.68), 1.569 (0.51), 1.585 (0.44), 1.603 (3.06), 1.610 (2.94), 1.615 (2.29), 1.636 (2.70), 1.639 (2.67), 1.642 (2.59), 1.646 (2.24), 2.522 (0.42), 2.729 (2.11), 2.843 (2.22), 2.850 (2.31), 2.874 (4.39), 2.881 (4.39), 2.888 (2.91), 2.905 (2.43), 2.912 (2.14), 3.541 (1.68), 3.551 (3.31), 3.559 (2.16), 3.572 (1.51), 3.583 (3.06), 3.593 (1.47), 6.400 (8.91), 7.950 (0.41).

LC-MS (Method 3): Rₜ=0.82 min; MS (ESlpos): m/z=199 [M+H]⁺.

Intermediate 12

1-(5-amino-1,3,4-thiadiazol-2-yl)-4-methylpiperidin-4-ol

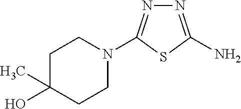

1.00 g (5.55 mmol, 1.0 equiv.) of 5-bromo-1,3,4-thiadiazol-2-amine was provided in 12 mL of DMF, 832 mg (7.22 mmol, 1.3 equiv.) of 4-methylpiperidin-4-ol and 1.54 g (11.1 mmol, 2.0 equiv.) of potassium carbonate were added, and the mixture was stirred at 80° C. over night. After filtration, the remaining solution was concentrated, 1.47 g of the title compound were obtained, which were used without further purification.

¹H-NMR (400 MHz, DMSO-d₆) δ [ppm]: 1.065 (1.32), 1.134 (16.00), 1.470 (0.52), 1.481 (0.56), 1.491 (2.71), 1.493 (3.07), 1.503 (4.66), 1.516 (2.56), 1.528 (1.73), 2.729 (1.38), 2.888 (1.64), 3.197 (0.59), 3.209 (0.58), 3.219 (0.65), 3.229 (1.74), 3.241 (1.35), 3.251 (1.75), 3.264 (2.19), 3.278 (2.85), 3.288 (1.56), 3.290 (1.30), 3.297 (0.76), 3.309 (1.28), 3.324 (7.26), 4.375 (2.48), 6.393 (5.28).

LC-MS (Method 3): Rₜ=0.53 min; MS (ESlpos): m/z=215 [M+H]⁺.

Intermediate 13

[4-(5-amino-1,3,4-thiadiazol-2-yl)piperazin-1-yl](cyclopropyl)methanone

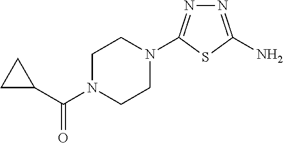 [SMILES: Nc1nnc(N2CCN(C(=O)C3CC3)CC2)s1]

100 g (5.24 mmol, 10 equiv.) of cyclopropyl(piperazin-1-yl)methanone hydrochloride (1:1) was provided in 11.7 mL of DMF, 1.27 g (6.82 mmol, 1.3 equiv.) of 5-bromo-1,3,4-thiadiazol-2-amine and 1.45 g (10.5 mmol, 2.0 equiv.) of potassium carbonate were added, and the mixture was stirred at 80° C. over night. After filtration, the remaining solution was concentrated, 1.36 g (89% of theory) of the title compound were obtained, which were used without further purification.

¹H-NMR (400 MHz, DMSO-d₆) δ [ppm]: 0.692 (0.49), 0.704 (1.60), 0.711 (4.03), 0.717 (2.17), 0.724 (1.79), 0.731 (5.65), 0.737 (5.13), 0.744 (3.64), 0.749 (4.56), 0.756 (1.79), 1.961 (0.44), 1.973 (0.91), 1.980 (0.93), 1.985 (0.66), 1.992 (1.67), 1.999 (0.68), 2.005 (0.92), 2.012 (0.85), 2.523 (0.53), 2.730 (12.54), 2.879 (0.54), 2.889 (16.00), 3.195 (1.18), 3.281 (1.22), 3.558 (1.06), 3.775 (1.05), 6.514 (6.80), 7.951 (1.94).

LC-MS (Method 3): Rₜ=0.57 min; MS (ESlpos): m/z=254 [M+H]⁺.

Intermediate 14

2-[1-(5-amino-1,3,4-thiadiazol-2-yl)piperidin-4-yl]propan-2-ol

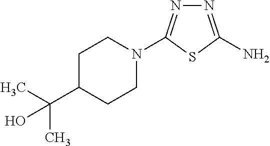 [SMILES: CC(C)(O)C1CCN(c2nnc(N)s2)CC1]

1.00 g (6.98 mmol, 1.0 equiv.) of 2-(piperidin-4-yl)propan-2-ol was provided in 15.6 mL of DMF, 1.68 g (9.08 mmol, 1.3 equiv.) of 5-bromo-1,3,4-thiadiazol-2-amine and 1.93 g (14.0 mmol, 2.0 equiv.) of potassium carbonate were added, and the mixture was stirred at 80° C. over night. After filtration, the remaining solution was concentrated, 0.60 g (36% of theory) of the title compound were obtained, which were used without further purification.

¹H-NMR (400 MHz, DMSO-d₆) δ [ppm]: 1.034 (16.00), 1.239 (0.40), 1.250 (0.44), 1.271 (0.59), 1.281 (0.61), 1.314 (0.75), 1.320 (0.40), 1.700 (0.63), 1.707 (0.73), 1.733 (0.68), 1.735 (0.72), 2.785 (0.52), 2.809 (0.73), 2.814 (0.89), 2.816 (0.89), 2.840 (0.43), 2.846 (0.43), 3.638 (0.77), 3.645 (0.53), 3.664 (0.58), 3.669 (0.70), 4.149 (3.41), 6.400 (2.70).

LC-MS (Method 3): Rₜ=0.62 min; MS (ESIpos): m/z=243 [M+H]⁺.

Intermediate 15

5-(4-methoxypiperidin-1-yl)-1,3,4-thiadiazol-2-amine

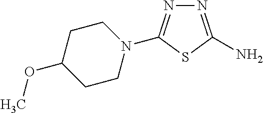

1.00 g (5.55 mmol, 1.0 equiv.) of 5-bromo-1,3,4-thiadiazol-2-amine was provided in 12 mL of DMF, 832 mg (7.22 mmol, 1.3 equiv.) of 4-methoxypiperidine and 1.54 g (11.1 mmol, 2.0 equiv.) of potassium carbonate were added, and the mixture was stirred at 80° C. over night. After filtration, the remaining solution was concentrated, 0.78 g (65% of theory) of the title compound were obtained, which were used without further purification.

¹H-NMR (400 MHz, DMSO-d₆) δ [ppm]: 1.461 (0.58), 1.472 (0.91), 1.482 (0.70), 1.494 (0.97), 1.504 (0.67), 1.516 (0.41), 1.853 (0.67), 1.859 (0.66), 1.865 (0.67), 1.869 (0.64), 1.877 (0.57), 1.882 (0.54), 1.885 (0.57), 1.892 (0.57), 1.898 (0.58), 2.729 (0.76), 2.889 (0.92), 3.019 (0.67), 3.028 (0.73), 3.042 (0.73), 3.051 (1.41), 3.060 (0.87), 3.074 (0.86), 3.083 (0.76), 3.254 (16.00), 3.354 (0.65), 3.363 (0.88), 3.373 (0.56), 3.384 (0.43), 3.417 (0.66), 3.427 (0.92), 3.432 (0.82), 3.441 (0.64), 3.445 (0.65), 3.449 (0.60), 3.462 (0.73), 3.464 (0.70), 3.474 (0.54), 6.419 (3.05).

LC-MS (Method 3): Rₜ=0.63 min; MS (ESlpos): m/z=215 [M+H]⁺.

Intermediate 16

5-(4,4-dimethylpiperidin-1-yl)-1,3,4-thiadiazol-2-amine

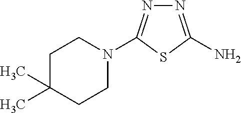

1.00 g (5.55 mmol, 1.0 equiv.) of 5-bromo-1,3,4-thiadiazol-2-amine was provided in 12 mL of DMF, 817 mg (7.22 mmol, 1.3 equiv.) of 4,4-dimethylpiperidine and 1.54 g (11.1 mmol, 2.0 equiv.) of potassium carbonate were added, and the mixture was stirred at 80° C. over night. After filtration, the remaining solution was concentrated, 1.27 g of the title compound were obtained, which were used without further purification.

¹H-NMR (400 MHz, DMSO-d₆) δ [ppm]: 0.940 (16.00), 1.358 (1.86), 1.368 (1.43), 1.373 (2.27), 1.378 (1.51), 1.387 (1.93), 3.209 (1.93), 3.218 (1.44), 3.223 (2.12), 3.228 (1.58), 3.238 (1.92), 6.395 (2.29).

LC-MS (Method 3): Rₜ=0.90 min; MS (ESIpos): m/z=213 [M+H]⁺.

### EXAMPLES

### Example 1

3-{[(4-methylpiperazin-1-yl)pacetyl]amino}-N-[5-(piperidin-1-yl)-1,3,4-thiadiazol-2-yl]-4-(trifluoromethoxy)benzamide

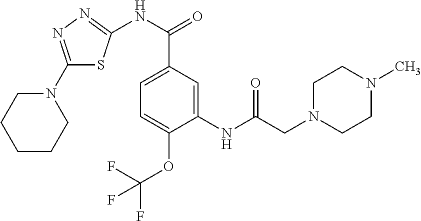 [SMILES: CN1CCN(CC(=O)Nc2cc(C(=O)Nc3nnc(N4CCCCC4)s3)ccc2OC(F)(F)F)CC1]

To a solution of the compound of intermediate 2 (150 mg, 0.32 mmol, 1.0 equiv.) and of the compound of intermediate 8 (118 mg, 0.64 mmol, 2.0 equiv.) in DMF (2 mL) was added (benzotriazol-1-yloxy)tripyrrolidinophosphonium hexafluorophosphate (PYBOP, 334 mg, 0.64 mmol, 2.0 equiv.) and diisopropylethylamine (0.28 mL, 1.60 mmol, 5.0 equiv.). The resulting mixture was stirred at room temperature over night. The precipitate was filtered off and dried. Purification by MPLC (Biotage Isolera; silica gel; dichloromethane/methanol gradient) yielded 35.4 mg (20% of theory) of the title compound.

¹H-NMR (400 MHz, DMSO-d₆) δ [ppm]: 1.036 (0.71), 1.054 (1.38), 1.071 (0.70), 1.599 (6.64), 1.607 (10.09), 1.729 (0.45), 1.907 (0.83), 2.187 (16.00), 2.318 (0.48), 2.322 (0.74), 2.327 (0.92), 2.331 (0.82), 2.336 (0.65), 2.350 (0.67), 2.411 (1.10), 2.523 (4.43), 2.539 (2.31), 2.585 (2.70), 2.659 (0.45), 2.664 (0.66), 2.669 (0.79), 2.673 (0.62), 2.729 (1.80), 2.889 (2.26), 3.205 (11.19), 3.404 (3.51), 3.417 (5.94), 3.426 (4.01), 5.755 (6.59), 7.578 (0.47), 7.583 (1.44), 7.587 (1.57), 7.592 (0.69), 7.600 (0.75), 7.604 (1.75), 7.608 (1.67), 7.908 (2.24), 7.914 (2.39), 7.930 (1.99), 7.935 (2.13), 7.950 (0.43), 8.930 (3.39), 8.936 (3.69), 9.914 (3.38).

LC-MS (Method 3): Rₜ=0.76 min; MS (ESlpos): m/z=528 [M+H]⁺.

### Example 2

tert-butyl 4-(54[3-{[(4-methylpiperazin-1-yl)pacetyl]amino}-4-(trifluoromethoxy)benzoyl]amino}-1,3,4-thiadiazol-2-yl)piperazine-1-carboxylate

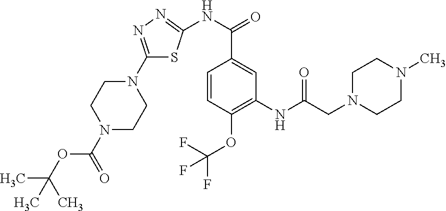 [SMILES: CN1CCN(CC(=O)Nc2cc(C(=O)Nc3nnc(N4CCN(C(=O)OC(C)(C)C)CC4)s3)ccc2OC(F)(F)F)CC1]

To a solution of the compound of intermediate 6 (150 mg, 0.41 mmol, 1.0 equiv.) and of the compound of intermediate 9 (238 mg, 0.74 mmol, 1.8 equiv.) in DMF (1.9 mL) was added (benzotriazol-1-yloxy)tripyrrolidinophosphonium hexafluorophosphate (PYBOP, 425 mg, 0.82 mmol, 2.0 equiv.) and diisopropylethylamine (0.36 mL, 2.04 mmol, 5.0 equiv.). The resulting mixture was stirred at room temperature over night. Water was added and the resulting mixture stood over night. The precipitate was filtered off and dried to yield 148 mg (55% of theory) of the title compound.

¹H-NMR (400 MHz, DMSO-d₆) δ [ppm]: 1.410 (0.85), 1.425 (16.00), 1.729 (0.61), 2.190 (4.16), 2.523 (1.76), 2.584 (1.05), 2.669 (0.46), 3.007 (0.46), 3.017 (0.47), 3.207 (2.89), 3.394 (0.67), 3.406 (1.38), 3.414 (1.29), 3.421 (1.46), 3.461 (1.25), 3.468 (1.13), 3.476 (1.23), 3.487 (0.69), 7.591 (0.40), 7.608 (0.46), 7.612 (0.42), 7.915 (0.63), 7.921 (0.62), 7.936 (0.51), 7.942 (0.55), 8.932 (0.92), 8.938 (0.92), 9.914 (0.93).

LC-MS (Method 3): Rₜ=0.81 min; MS (ESlpos): m/z=628 [M+H]⁺.

### Example 3

tert-butyl 4-[5-({3-[(morpholin-4-ylacetyl)amino]-4-(trifluoromethoxy)benzoyl}amino)-1,3,4-thiadiazol-2-yl]piperazine-1-carboxylate

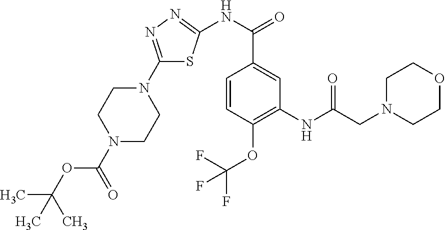 [SMILES: CC(C)(C)OC(=O)N1CCN(c2nnc(NC(=O)c3ccc(OC(F)(F)F)c(NC(=O)CN4CCOCC4)c3)s2)CC1]

To a solution of the compound of intermediate 7 (170 mg, 0.44 mmol, 1.0 equiv.) and of the compound of intermediate 9 (256 mg, 0.79 mmol, 1.8 equiv.) in DMF (2.0 mL) was added (benzotriazol-1-yloxy)tripyrrolidinophosphonium hexafluorophosphate (PYBOP, 457 mg, 0.88 mmol, 2.0 equiv.) and diisopropylethylamine (0.38 mL, 2.20 mmol, 5.0 equiv.). The resulting mixture was stirred at room temperature over night. After filtration, purification of half of the solution by HPLC (method 5) and MPLC (Biotage Isolera; silica gel; ethyl acetate/methanol gradient) yielded 52.0 mg (19% of theory) of the title compound.

¹H-NMR (400 MHz, DMSO-d₆) δ [ppm]: 1.425 (16.00), 2.523 (1.35), 2.561 (1.36), 2.573 (1.70), 2.584 (1.35), 3.223 (2.89), 3.400 (0.55), 3.411 (1.16), 3.419 (1.13), 3.426 (1.30), 3.462 (1.22), 3.469 (1.07), 3.478 (1.11), 3.489 (0.59), 3.634 (1.07), 3.645 (1.44), 3.657 (1.08), 7.931 (0.61), 7.936 (0.61), 7.952 (0.49), 7.958 (0.54), 8.874 (0.42), 9.903 (0.72).

LC-MS (Method 3): Rₜ=0.80 min; MS (ESlpos): m/z=616 [M+H]⁺.

### Example 4

3-{[(4-methylpiperazin-1-yl)pacetyl]amino}-N-[5-(pyrrolidin-1-yl)-1,3,4-thiadiazol-2-yl]-4-(trifluoromethoxy)benzamide

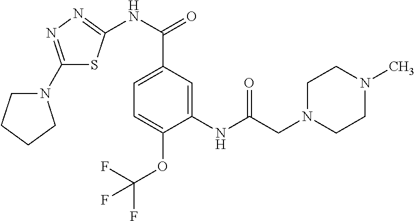 [SMILES: CN1CCN(CC(=O)Nc2cc(C(=O)Nc3nnc(N4CCCC4)s3)ccc2OC(F)(F)F)CC1]

To a solution of the compound of intermediate 6 (150 mg, 0.41 mmol, 1.0 equiv.) and of 5-(pyrrolidin-1-yl)-1,3,4-thiadiazol-2-amine (83.4 mg, 0.49 mmol, 1.2 equiv.) in DMF (2.5 mL) was added (benzotriazol-1-yloxy)tripyrrolidinophosphonium hexafluorophosphate (PYBOP, 638 mg, 1.23 mmol, 3.0 equiv.) and diisopropylethylamine (0.29 mL, 1.63 mmol, 4.0 equiv.). The resulting mixture was stirred at room temperature for 4 days. Water was added and the precipitate was filtered off, dried and purified by HPLC (mobile phase: acetonitrile/water+0.1% ammonia) to yield 56.8 mg (27% of theory) of the title compound.

¹H-NMR (400 MHz, DMSO-d₆) δ [ppm]: 1.232 (0.76), 1.907 (0.87), 1.963 (2.73), 1.971 (3.45), 1.980 (7.77), 1.988 (3.41), 1.996 (2.92), 2.177 (16.00), 2.317 (1.06), 2.322 (2.12), 2.326 (2.73), 2.331 (2.12), 2.336 (1.33), 2.381 (1.06), 2.523 (4.47), 2.583 (2.24), 2.659 (0.83), 2.664 (1.82), 2.669 (2.43), 2.673 (1.78), 2.678 (0.76), 3.202 (10.81), 3.388 (2.96), 3.404 (7.51), 3.421 (2.69), 7.574 (1.21), 7.576 (1.18), 7.594 (1.33), 7.595 (1.33), 7.907 (2.01), 7.913 (2.05), 7.928 (1.71), 7.934 (1.78), 8.936 (3.18), 8.942 (3.07), 9.907 (3.00).

LC-MS (Method 3): Rₜ=0.77 min; MS (ESlpos): m/z=514 [M+H]⁺.

### Example 5

N-(5-cyclohexyl-1,3,4-thiadiazol-2-yl)-3-{[(4-methylpiperazin-1-yl)pacetyl]amino}-4-(trifluoromethoxy)benzamide

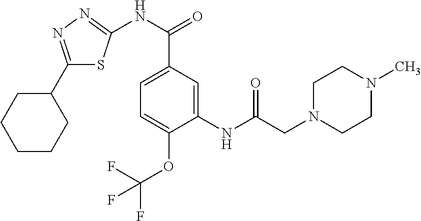 [SMILES: CN1CCN(CC(=O)Nc2cc(C(=O)Nc3nnc(C4CCCCC4)s3)ccc2OC(F)(F)F)CC1]

To a solution of the compound of intermediate 6 (150 mg, 0.41 mmol, 1.0 equiv.) and of 5-cyclohexyl-1,3,4-thiadiazol-2-amine (97.3 mg, 0.53 mmol, 1.3 equiv.) in DMF (2.5 mL) was added (benzotriazol-1-yloxy)tripyrrolidinophosphonium hexafluorophosphate (PYBOP, 637 mg, 1.23 mmol, 3.0 equiv.) and diisopropylethylamine (0.29 mL, 1.63 mmol, 4.0 equiv.). The resulting mixture was stirred at room temperature for 4 days. Water was added and the precipitate was filtered off and dried to yield 112 mg (47% of theory) of the title compound.

¹H-NMR (400 MHz, DMSO-d₆) δ [ppm]: 1.140 (0.51), 1.162 (0.85), 1.171 (0.61), 1.193 (1.10), 1.223 (0.61), 1.268 (0.72), 1.299 (1.75), 1.307 (1.26), 1.331 (1.75), 1.338 (1.13), 1.362 (0.85), 1.396 (0.82), 1.426 (1.69), 1.428 (1.59), 1.433 (1.64), 1.455 (1.51), 1.462 (1.52), 1.492 (0.51), 1.576 (0.93), 1.608 (0.85), 1.637 (0.77), 1.667 (1.33), 1.677 (1.90), 1.709 (1.55), 1.941 (1.85), 1.974 (1.51), 2.063 (1.83), 2.107 (16.00), 2.229 (0.88), 2.234 (1.18), 2.238 (0.92), 2.429 (3.37), 2.571 (0.80), 2.576 (1.08), 2.580 (0.75), 2.585 (0.47), 2.638 (2.04), 2.797 (2.50), 2.916 (0.54), 2.925 (0.54), 2.947 (0.83), 2.966 (0.88), 2.976 (1.55), 2.984 (0.82), 3.003 (0.74), 3.120 (11.68), 7.506 (0.59), 7.511 (1.47), 7.515 (1.62), 7.528 (0.75), 7.532 (1.78), 7.537 (1.67), 7.853 (2.50), 7.859 (2.67), 7.875 (2.03), 7.880 (2.13), 8.860 (3.70), 8.865 (3.71), 9.826 (3.44).

LC-MS (Method 3): Rₜ=0.80 min; MS (ESIpos): m/z=527 [M+H]⁺.

### Example 6

methyl 4-(5-{[3-{[(4-methylpiperazin-1-yl)pacetyl]amino}-4-(trifluoromethoxy)benzoyl]amino}-1,3,4-thiadiazol-2-yl)ppiperazine-1-carboxylate

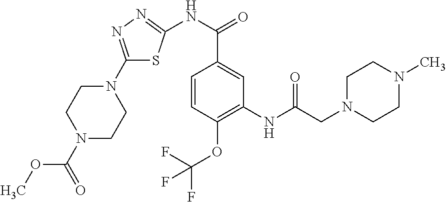

To a solution of the compound of intermediate 6 (150 mg, 0.41 mmol, 1.0 equiv.) and of the compound of intermediate 10 (144 mg, 0.53 mmol, 1.3 equiv.) in DMF (2.1 mL) was added (benzotriazol-1-yloxy)tripyrrolidinophosphonium hexafluorophosphate (PYBOP, 531 mg, 1.02 mmol, 2.5 equiv.) and diisopropylethylamine (0.32 mL, 1.84 mmol, 4.5 equiv.). The resulting mixture was stirred at room temperature over night. After filtration, purification of half of the solution by HPLC (mobile phase: acetonitrile/water+0.1% ammonia) yielded 55.6 mg (23% of theory) of the title compound.

¹H-NMR (400 MHz, DMSO-d₆) δ [ppm]: 2.192 (10.03), 2.323 (0.66), 2.327 (0.87), 2.332 (0.66), 2.337 (0.44), 2.406 (0.65), 2.523 (1.50), 2.586 (1.44), 2.665 (0.60), 2.669 (0.77), 2.674 (0.56), 3.207 (7.56), 3.423 (1.66), 3.434 (3.19), 3.442 (2.71), 3.449 (3.32), 3.461 (0.52), 3.500 (0.61), 3.511 (3.01), 3.518 (2.51), 3.526 (3.04), 3.537 (1.64), 3.635 (16.00), 7.588 (0.93), 7.592 (0.99), 7.605 (0.45), 7.609 (1.09), 7.614 (1.01), 7.915 (1.65), 7.921 (1.57), 7.936 (1.32), 7.942 (1.38), 8.931 (2.32), 8.936 (2.26), 9.914 (2.34).

LC-MS (Method 3): Rₜ=0.70 min; MS (ESlpos): m/z=587 [M+H]⁺.

### Example 7

3-{[(4-methylpiperazin-1-yl)pacetyl]amino}-N-[5-(4-methylpiperidin-1-yl-1,3,4-thiadiazol-2-yl]-4-(trifluoromethoxy)benzamide

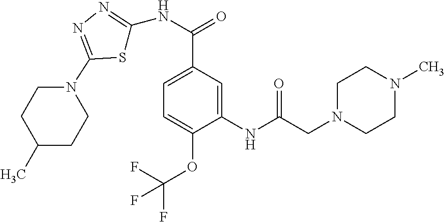 [SMILES: CC1CCN(c2nnc(NC(=O)c3ccc(OC(F)(F)F)c(NC(=O)CN4CCN(C)CC4)c3)s2)CC1]

To a solution of the compound of intermediate 6 (150 mg, 0.41 mmol, 1.0 equiv.) and of the compound of intermediate 11 (105 mg, 0.53 mmol, 1.3 equiv.) in DMF (3.0 mL) was added (benzotriazol-1-yloxy)tripyrrolidinophosphonium hexafluorophosphate (PYBOP, 425 mg, 0.82 mmol, 2.0 equiv.) and diisopropylethylamine (0.28 mL, 1.63 mmol, 4.0 equiv.). The resulting mixture was stirred at room temperature for 5 days. Water and ethanol were added and the resulting mixture was stirred for 20 minutes. The precipitate was filtered off and dried to yield 57.0 mg (26% of theory) of the title compound.

¹H-NMR (400 MHz, DMSO-d₆) δ [ppm]: 0.925 (7.72), 0.941 (7.74), 1.171 (0.44), 1.192 (1.06), 1.202 (1.08), 1.220 (1.18), 1.231 (1.26), 1.251 (0.58), 1.262 (0.51), 1.587 (0.46), 1.596 (0.56), 1.605 (0.52), 1.613 (0.52), 1.622 (0.45), 1.669 (1.55), 1.677 (1.45), 1.702 (1.39), 1.705 (1.36), 1.712 (1.32), 1.728 (0.44), 2.182 (16.00), 2.317 (0.45), 2.322 (0.71), 2.326 (0.88), 2.331 (0.75), 2.336 (0.57), 2.389 (0.93), 2.523 (1.52), 2.584 (2.12), 2.664 (0.59), 2.668 (0.72), 2.673 (0.54), 3.006 (1.30), 3.013 (1.33), 3.038 (2.18), 3.044 (2.14), 3.069 (1.24), 3.076 (1.08), 3.202 (10.89), 3.785 (1.00), 3.795 (1.76), 3.803 (1.15), 3.817 (0.97), 3.827 (1.63), 3.835 (0.96), 7.580 (1.39), 7.584 (1.34), 7.597 (0.73), 7.601 (1.66), 7.605 (1.40), 7.905 (2.24), 7.911 (2.19), 7.927 (1.79), 7.932 (1.88), 8.929 (3.41), 8.934 (3.32), 9.912 (3.04).

LC-MS (Method 3): Rₜ=0.85 min; MS (ESlpos): m/z=542 [M+H]⁺.

### Example 8

N-[5-(4-hydroxy-4-methylpiperidin-1-yl)-1,3,4-thiadiazol-2-yl]-3-{[(4-methylpiperazin-1-yl)pacetyl]amino}-4-(trifluoromethoxy)benzamide

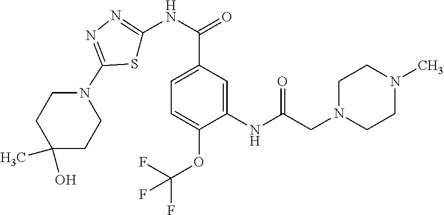

To a solution of the compound of intermediate 6 (150 mg, 0.41 mmol, 1.0 equiv.) and of the compound of intermediate 12 (114 mg, 0.53 mmol, 1.3 equiv.) in DMF (3.0 mL) was added (benzotriazol-1-yloxy)tripyrrolidinophosphonium hexafluorophosphate (PYBOP, 425 mg, 0.82 mmol, 2.0 equiv.) and diisopropylethylamine (0.28 mL, 1.63 mmol, 4.0 equiv.). The resulting mixture was stirred at room temperature over night. Water and ethanol were added and the resulting mixture was extracted with dichloromethane. The organic phase was dried over sodium sulfate, filtered and dried. Purification by HPLC (method 5) yielded 65.8 mg (27% of theory) of the title compound.

¹H-NMR (400 MHz, DMSO-d₆) δ [ppm]: 1.164 (15.33), 1.551 (3.08), 1.563 (4.70), 1.575 (2.92), 1.585 (1.70), 2.180 (16.00), 2.317 (0.54), 2.322 (0.91), 2.327 (1.21), 2.331 (0.99), 2.336 (0.67), 2.393 (1.01), 2.523 (2.53), 2.539 (0.75), 2.583 (2.23), 2.664 (0.73), 2.669 (0.96), 2.674 (0.69), 3.201 (10.93), 3.355 (2.15), 3.369 (1.60), 3.371 (1.40), 3.378 (1.48), 3.387 (1.88), 3.401 (1.34), 3.410 (1.61), 3.424 (1.17), 3.506 (0.77), 3.508 (1.04), 3.519 (2.23), 3.530 (1.21), 3.540 (0.86), 3.551 (1.55), 3.561 (0.72), 4.444 (5.55), 7.569 (0.47), 7.574 (1.30), 7.578 (1.37), 7.582 (0.51), 7.590 (0.60), 7.595 (1.58), 7.600 (1.37), 7.904 (2.20), 7.910 (2.20), 7.926 (1.86), 7.932 (1.94), 8.930 (3.43), 8.936 (3.47), 9.904 (2.95).

LC-MS (Method 3): Rₜ=0.66 min; MS (ESlpos): m/z=558 [M+H]⁺.

### Example 9

N-{5-[4-(cyclopropylcarbonyl)piperazin-1-yl]-1,3,4-thiadiazol-2-yl1-3-{[(4-methylpiperazin-1-yl)pacetyl]amino}-4-(trifluoromethoxy)benzamide

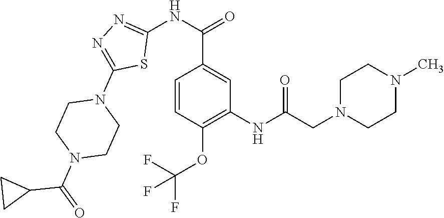 [SMILES: CN1CCN(CC(=O)Nc2cc(C(=O)Nc3nnc(N4CCN(C(=O)C5CC5)CC4)s3)ccc2OC(F)(F)F)CC1]

To a solution of the compound of intermediate 6 (150 mg, 0.41 mmol, 1.0 equiv.) and of the compound of intermediate 13 (150 mg, 0.53 mmol, 1.3 equiv.) in DMF (2.1 mL) was added (benzotriazol-1-yloxy)tripyrrolidinophosphonium hexafluorophosphate (PYBOP, 531 mg, 1.02 mmol, 2.5 equiv.) and diisopropylethylamine (0.32 mL, 1.84 mmol, 4.5 equiv.). The resulting mixture was stirred at room temperature over night. After filtration, purification of half of the solution by HPLC (mobile phase: acetonitrile/water+0.1% ammonia) yielded 85.0 mg (33% of theory) of the title compound.

¹H-NMR (400 MHz, DMSO-d6) δ [ppm]: 0.709 (0.44), 0.721 (1.36), 0.729 (3.18), 0.734 (1.95), 0.741 (1.52), 0.749 (4.20), 0.756 (3.81), 0.762 (2.98), 0.768 (3.58), 0.775 (1.53), 1.752 (1.68), 2.003 (0.77), 2.009 (0.82), 2.014 (0.63), 2.022 (1.38), 2.028 (0.60), 2.034 (0.76), 2.041 (0.69), 2.102 (1.49), 2.183 (16.00), 2.317 (0.42), 2.322 (0.65), 2.326 (0.82), 2.331 (0.71), 2.336 (0.55), 2.400 (0.97), 2.466 (0.48), 2.522 (1.57), 2.539 (0.78), 2.582 (2.18), 2.664 (0.52), 2.668 (0.64), 2.673 (0.49), 3.204 (10.73), 3.409 (1.38), 3.493 (1.27), 3.624 (1.12), 3.844 (1.06), 7.576 (0.44), 7.580 (1.31), 7.585 (1.40), 7.589 (0.54), 7.597 (0.65), 7.602 (1.57), 7.606 (1.43), 7.916 (2.21), 7.922 (2.19), 7.938 (1.78), 7.943 (1.93), 8.939 (3.39), 8.945 (3.42), 9.911 (3.26).

LC-MS (Method 3): Rₜ=0.68 min; MS (ESIpos): m/z=597 [M+H]⁺.

Example 10

N-{5-[4-(2-hydroxypropan-2-yl)piperidin-1-yl]-1,3,4-thiadiazol-2-yl1-3-[(morpholin-4-ylacetyl)amino]-4-(trifluoromethoxy)benzamide

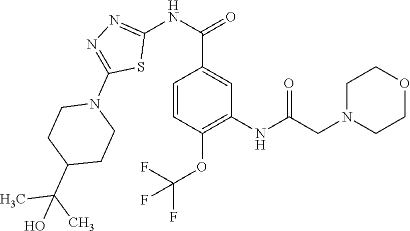 [SMILES: CC(C)(O)C1CCN(c2nnc(NC(=O)c3ccc(OC(F)(F)F)c(NC(=O)CN4CCOCC4)c3)s2)CC1]

To a solution of the compound of intermediate 7 (170 mg, 0.44 mmol, 1.0 equiv.) and of the compound of intermediate 14 (128 mg, 0.53 mmol, 1.2 equiv.) in DMF (2.2 mL) was added (benzotriazol-1-yloxy)tripyrrolidinophosphonium hexafluorophosphate (PYBOP, 457 mg, 0.88 mmol, 2.0 equiv.) and diisopropylethylamine (0.31 mL, 1.76 mmol, 4.0 equiv.). The resulting mixture was stirred at room temperature for 2 days. Water was added and the resulting precipitate was filtered off and dried to yield 193 mg (77% of theory) of the title compound.

¹H-NMR (400 MHz, DMSO-d₆) δ [ppm]: 1.052 (16.00), 1.287 (0.51), 1.298 (0.57), 1.319 (0.70), 1.329 (0.74), 1.394 (0.49), 1.424 (0.48), 1.778 (0.91), 1.805 (0.79), 1.809 (0.82), 2.522 (0.59), 2.560 (1.87), 2.572 (2.63), 2.584 (1.99), 2.943 (0.46), 2.950 (0.58), 2.974 (0.98), 2.979 (1.02), 2.981 (1.03), 3.006 (0.59), 3.013 (0.49), 3.221 (5.90), 3.633 (2.09), 3.645 (2.85), 3.656 (2.05), 3.886 (0.88), 3.918 (0.81), 4.186 (4.42), 7.588 (0.62), 7.593 (0.64), 7.610 (0.71), 7.614 (0.66), 7.923 (1.16), 7.929 (1.14), 7.945 (0.95), 7.950 (1.04), 8.869 (1.05), 8.871 (1.08), 8.874 (1.06), 9.898 (1.62).

LC-MS (Method 3): Rₜ=0.71 min; MS (ESlpos): m/z=573 [M+H]⁺.

### Example 11

N-{5-(4-methoxypiperidin-1-yl)-1,3,4-thiadiazol-2-yl]-3-{[(4-methylpiperazin-1-yl)acetyl]amino}-4-(trifluoromethoxy)benzamide

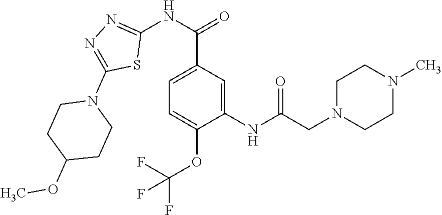

To a solution of the compound of intermediate 6 (150 mg, 0.41 mmol, 1.0 equiv.) and of intermediate 15 (114 mg, 0.53 mmol, 1.3 equiv.) in DMF (2.5 mL) was added (benzotriazol-1-yloxy) tripyrrolidinophosphonium hexafluorophosphate (PYBOP, 425 mg, 0.82 mmol, 2.0 equiv.) and diisopropylethylamine (0.29 mL, 1.63 mmol, 4.0 equiv.). The resulting mixture was stirred at room temperature for 4 hours. Water and ethanol were added, the resulting mixture was stirred for 30 minutes and the precipitate was filtered off and dried. Purification by HPLC (method 5) yielded 64.0 mg (27% of theory) of the title compound.

¹H-NMR (400 MHz, DMSO-d₆) δ [ppm]: 1.522 (0.48), 1.533 (0.74), 1.543 (0.60), 1.554 (0.79), 1.565 (0.55), 1.907 (0.84), 1.914 (0.63), 1.922 (0.62), 1.931 (0.57), 1.938 (0.52), 1.947 (0.53), 1.954 (0.51), 2.181 (8.12), 2.323 (0.47), 2.327 (0.60), 2.332 (0.50), 2.396 (0.51), 2.523 (1.32), 2.577 (1.15), 2.582 (1.17), 2.669 (0.51), 3.202 (5.57), 3.220 (0.73), 3.229 (0.81), 3.242 (0.86), 3.252 (1.43), 3.261 (1.15), 3.278 (16.00), 3.404 (0.66), 3.412 (0.73), 3.424 (0.74), 3.433 (0.86), 3.442 (0.63), 3.452 (0.51), 3.454 (0.49), 3.616 (0.58), 3.625 (0.76), 3.629 (0.82), 3.640 (0.63), 3.644 (0.62), 3.647 (0.61), 3.657 (0.60), 3.662 (0.65), 3.664 (0.63), 3.674 (0.45), 7.578 (0.71), 7.583 (0.73), 7.600 (0.84), 7.604 (0.75), 7.906 (1.13), 7.911 (1.12), 7.928 (0.94), 7.933 (0.97), 8.930 (1.75), 8.936 (1.77), 9.912 (1.55).

LC-MS (Method 3): Rₜ=0.70 min; MS (ESlpos): m/z=558 [M+H]⁺.

### Example 12

4-(2-{[5-{[5-(4,4-dimethylpiperidin-1-yl)-1,3,4-thiadiazol-2-yl]carbamoyl}-2-(trifluoromethoxy)phenyl]amino}-2-oxoethyl)-1-methylpiperazin-1-ium hexafluorophosphate

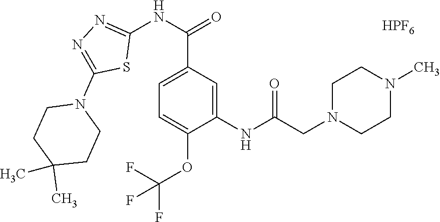

To a solution of the compound of intermediate 6 (150 mg, 0.41 mmol, 1.0 equiv.) and of the compound of intermediate 16 (113 mg, 0.53 mmol, 1.3 equiv.) in DMF (3.0 mL) was added (benzotriazol-1-yloxy)tripyrrolidinophosphonium hexafluorophosphate (PYBOP, 425 mg, 0.82 mmol, 2.0 equiv.) and diisopropylethylamine (0.28 mL, 1.63 mmol, 4.0 equiv.). The resulting mixture was stirred at room temperature for 4 days. Water and ethanol were added and the resulting precipitate was filtered off and dried. Purification by HPLC (mobile phase: acetonitrile/water+0.1% formic acid) yielded 63.1 mg (22% of theory) of the title compound.

¹H-NMR (400 MHz, DMSO-d₆) δ [ppm]: 0.978 (16.00), 1.417 (1.81), 1.427 (1.84), 1.432 (2.32), 1.437 (1.86), 1.446 (1.84), 1.713 (0.41), 1.721 (0.41), 1.729 (1.08), 1.738 (0.41), 1.746 (0.41), 2.327 (0.42), 2.523 (1.42), 2.659 (0.44), 2.665 (0.62), 2.669 (0.73), 2.674 (0.57), 2.804 (3.10), 2.991 (0.57), 3.001 (0.78), 3.007 (1.20), 3.012 (0.80), 3.017 (1.29), 3.024 (1.01), 3.034 (0.90), 3.377 (3.50), 3.415 (2.16), 3.424 (2.21), 3.429 (2.60), 3.434 (2.32), 3.444 (2.07), 7.581 (0.55), 7.585 (0.57), 7.602 (0.62), 7.607 (0.58), 7.977 (0.92), 7.983 (0.90), 7.999 (0.78), 8.004 (0.80), 8.618 (0.92), 8.622 (0.96), 8.625 (0.90), 9.796 (1.19).

LC-MS (Method 1): Rₜ=0.98 min; MS (ESlpos): m/z=556 [M+H]⁺.

The following examples were prepared in analogy to the described methods, supra.

| | | | Rt |
|---|---|---|---|
| Example | | | [min] |
| No | Structure | IUPAC Name | method |
| 13 | 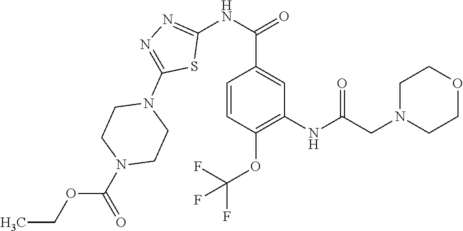 [SMILES: CCOC(=O)N1CCN(c2nnc(NC(=O)c3ccc(OC(F)(F)F)c(NC(=O)CN4CCOCC4)c3)s2)CC1] | ethyl 4-[5-({3-[(morpholin-4- yl-acetyl)amino]-4- (trifluoromethoxy)benzoyl}- amino)-1,3,4-thiadiazol-2- yl]piperazine-1-carboxylate | 0.713 |
| 14 | 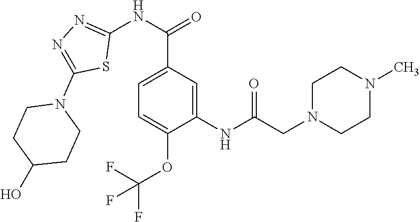 [SMILES: CN1CCN(CC(=O)Nc2cc(C(=O)Nc3nnc(N4CCC(O)CC4)s3)ccc2OC(F)(F)F)CC1] | N-[5-(4-hydroxypiperidin- 1-yl)-1,3,4-thiadiazol-2-yl]-3- {[(4-methylpiperazin-1- yl)acetyl]amino}-4- (trifluoromethoxy)benzamide | 0.643 |
| 15 | 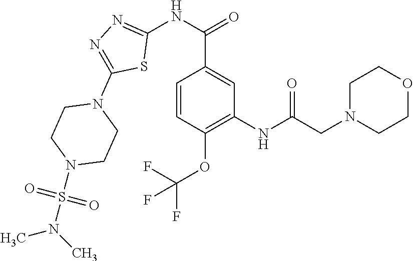 [SMILES: CN(C)S(=O)(=O)N1CCN(c2nnc(NC(=O)c3ccc(OC(F)(F)F)c(NC(=O)CN4CCOCC4)c3)s2)CC1] | N-{5-[4-(dimethylsulfamoyl)- piperazin-1-yl]-1,3,4-thiadiazol- 2-yl}-3-[(morpholin-4-ylacetyl)- amino]-4-(trifluoromethoxy) benzamide | 0.763 |
| 16 | 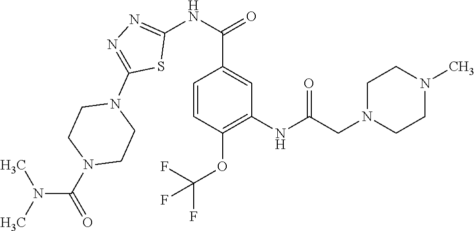 | N,N-dimethyl-4-(5-{[3-{[(4- methylpiperazin-1-yl)acetyl]- amino}-4-(trifluoromethoxy)- benzoyl]amino}-1,3,4- thiadiazol-2-yl)piperazine-1- carboxamide | 0.683 |
| 17 | 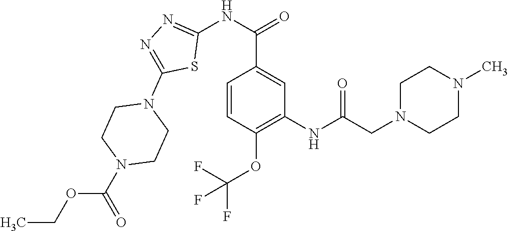 [SMILES: CCOC(=O)N1CCN(c2nnc(NC(=O)c3ccc(OC(F)(F)F)c(NC(=O)CN4CCN(C)CC4)c3)s2)CC1] | ethyl 4-(5-{[3-{[(4- methylpiperazin-1-yl)acetyl]- amino}-4-(trifluoromethoxy)- benzoyl]amino}-1,3,4- thiadiazol-2-yl)piperazine-1- carboxylate | 0.743 |
| 18 | 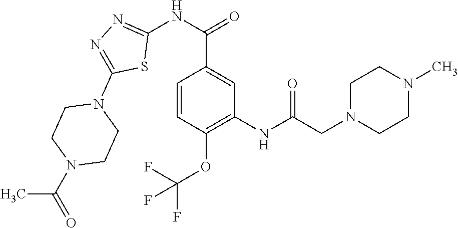 [SMILES: CC(=O)N1CCN(c2nnc(NC(=O)c3ccc(OC(F)(F)F)c(NC(=O)CN4CCN(C)CC4)c3)s2)CC1] | N-[5-(4-acetylpiperazin-1- yl)-1,3,4-thiadiazol-2-yl]-3- {[(4-methylpiperazin-1-yl)- acetyl]amino}-4- (trifluoromethoxy)benzamide | 0.643 |
| 19 | 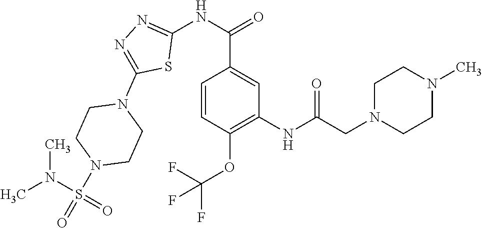 | N-{5-[4-(dimethylsulfamoyl)- piperazin-1-yl]-1,3,4- thiadiazol-2-yl}-3-{[(4- methylpiperazin-1-yl)acetyl]- amino}-4-(trifluoromethoxy)- benzamide | 0.713 |
| 20 | 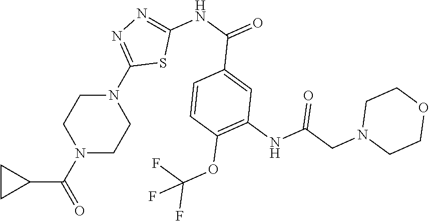 [SMILES: O=C(CN1CCOCC1)Nc1cc(C(=O)Nc2nnc(N3CCN(C(=O)C4CC4)CC3)s2)ccc1OC(F)(F)F] | N-{5-[4-(cyclopropylcarbonyl)- piperazin-1-yl]-1,3,4- thiadiazol-2-yl}-3-[(morpholin- 4-ylacetyl)amino]-4- (trifluoromethoxy)benzamide | 0.673 |
| 21 | 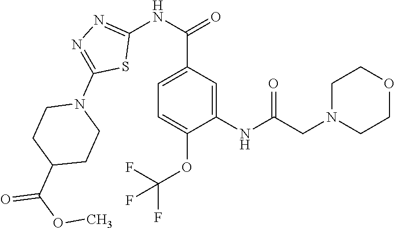 [SMILES: COC(=O)C1CCN(c2nnc(NC(=O)c3ccc(OC(F)(F)F)c(NC(=O)CN4CCOCC4)c3)s2)CC1] | methyl 1-[5-({3-[(morpholin- 4-ylacetyl)amino]-4- (trifluoromethoxy)benzoyl}- amino)-1,3,4-thiadiazol-2-yl]- piperidine-4-carboxylate | 0.693 |
| 22 | 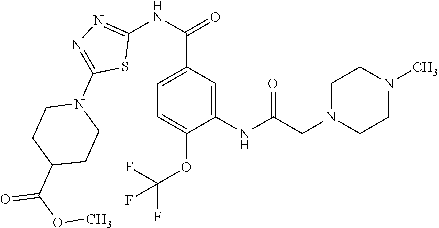 [SMILES: COC(=O)C1CCN(c2nnc(NC(=O)c3ccc(OC(F)(F)F)c(NC(=O)CN4CCN(C)CC4)c3)s2)CC1] | methyl 1-(5-{[3-{[(4-methyl- piperazin-1-yl)acetyl]amino}- 4-(trifluoromethoxy)benzoyl]- amino}-1,3,4-thiadiazol-2-yl)- piperidine-4-carboxylate | 0.723 |
| 23 | 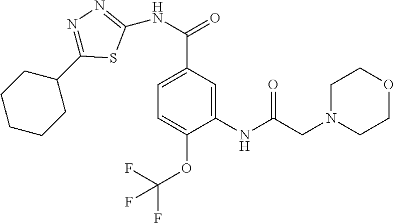 [SMILES: O=C(CN1CCOCC1)Nc1cc(C(=O)Nc2nnc(C3CCCCC3)s2)ccc1OC(F)(F)F] | N-(5-cyclohexyl-1,3,4- thiadiazol-2-yl)-3-[(morpholin- 4-ylacetyl)amino]-4- (trifluoromethoxy)benzamide | 1.234 |
| 24 | 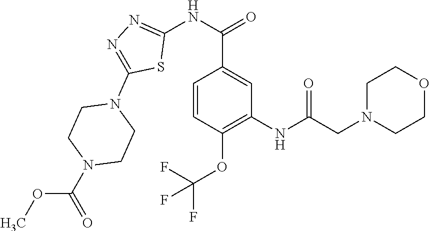 [SMILES: COC(=O)N1CCN(c2nnc(NC(=O)c3ccc(OC(F)(F)F)c(NC(=O)CN4CCOCC4)c3)s2)CC1] | methyl 4-[5-({3-[(morpholin- 4-ylacetyl)amino]-4- (trifluoromethoxy)benzoyl}- amino)-1,3,4-thiadiazol-2- yl]piperazine-1-carboxylate | 0.683 |
| 25 | 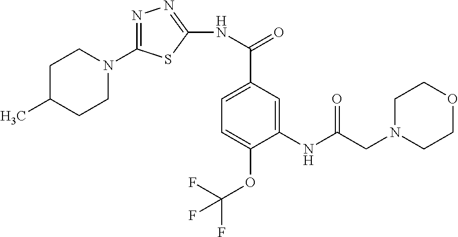 [SMILES: CC1CCN(c2nnc(NC(=O)c3ccc(OC(F)(F)F)c(NC(=O)CN4CCOCC4)c3)s2)CC1] | N-[5-(4-methylpiperidin-1- yl)-1,3,4-thiadiazol-2-yl]- 3-[(morpholin-4-ylacetyl)- amino]-4-(trifluoromethoxy)- benzamide | 1.104 |
| 26 | 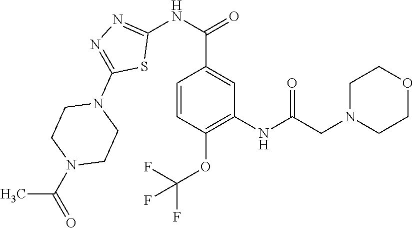 [SMILES: CC(=O)N1CCN(c2nnc(NC(=O)c3ccc(OC(F)(F)F)c(NC(=O)CN4CCOCC4)c3)s2)CC1] | N-[5-(4-acetylpiperazin-1-yl)- 1,3,4-thiadiazol-2-yl]-3- [(morpholin-4-ylacetyl)amino]- 4-(trifluoromethoxy)benzamide | 0.633 |
| 27 | 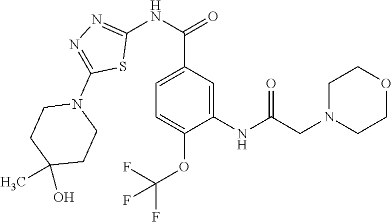 [SMILES: CC1(O)CCN(c2nnc(NC(=O)c3ccc(OC(F)(F)F)c(NC(=O)CN4CCOCC4)c3)s2)CC1] | N-[5-(4-hydroxy-4-methyl- piperidin-1-yl)-1,3,4- thiadiazol-2-yl]-3-[(morpholin- 4-ylacetyl)amino\|-4- (trifluoromethoxy)benzamide | 0.723 |
| 28 | 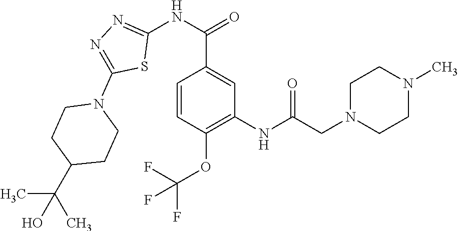 [SMILES: CN1CCN(CC(=O)Nc2cc(C(=O)Nc3nnc(N4CCC(C(C)(C)O)CC4)s3)ccc2OC(F)(F)F)CC1] | N-{5-[4-(2-hydroxypropan-2- yl)piperidin-1-yl]-1,3,4- thiadiazol-2-yI}-3- {[(4-methylpiperazin-1-yl)- acetyl]amino}-4- (trifluoromethoxy)benzamide | 0.733 |
| 29 | 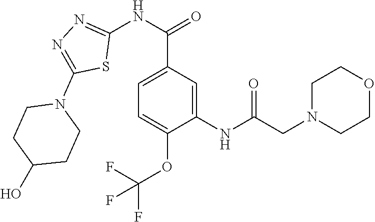 [SMILES: O=C(CN1CCOCC1)Nc1cc(C(=O)Nc2nnc(N3CCC(O)CC3)s2)ccc1OC(F)(F)F] | N-[5-(4-hydroxypiperidin- 1-yl)-1,3,4-thiadiazol-2-yl]- 3-[(morpholin-4-ylacetyl)- amino]-4-(trifluoromethoxy)- benzamide | 0.613 |
| 30 | 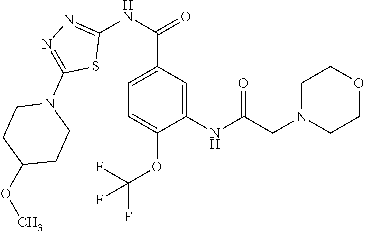 [SMILES: COC1CCN(c2nnc(NC(=O)c3ccc(OC(F)(F)F)c(NC(=O)CN4CCOCC4)c3)s2)CC1] | N-[5-(4-methoxypiperidin- 1-yl)-1,3,4-thiadiazol-2-yl]-3- ((morpholin-4-ylacetyl)- amino]-4-(trifluoromethoxy)- benzamide | 0.683 |
| 31 | 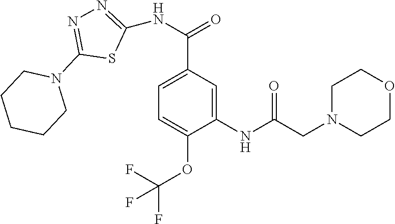 [SMILES: O=C(CN1CCOCC1)Nc1cc(C(=O)Nc2nnc(N3CCCCC3)s2)ccc1OC(F)(F)F] | 3-[(morpholin-4-ylacetyl)- amino]-N-[5-(piperidin-1-yl)- 1,3,4-thiadiazol-2-yl]-4- (trifluoromethoxy)benzamide | 1.024 |
| 32 | 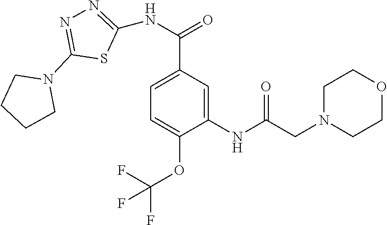 [SMILES: O=C(CN1CCOCC1)Nc1cc(C(=O)Nc2nnc(N3CCCC3)s2)ccc1OC(F)(F)F] | 3-[(morpholin-4-ylacetyl)- amino]-N-(5-(pyrrolidin-1-yl)- 1,3,4-thiadiazol-2-yl)-4- (trifluoromethoxy)benzamide | 0.934 |
| 33 | 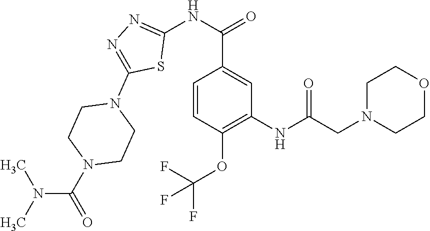 [SMILES: CN(C)C(=O)N1CCN(c2nnc(NC(=O)c3ccc(OC(F)(F)F)c(NC(=O)CN4CCOCC4)c3)s2)CC1] | N,N-dimethy1-4-[5-({3- [(morpholin-4-ylacetyl)amino- 4-(trifluoromethoxy)benzoyl}- amino)-1,3,4-thiadiazol-2-yl]- piperazine-1-carboxamide | 0.663 |
| 34 | 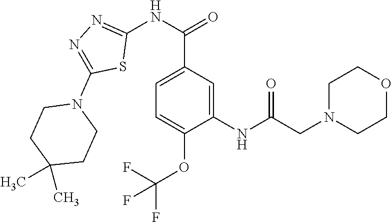 [SMILES: CC1(C)CCN(c2nnc(NC(=O)c3ccc(OC(F)(F)F)c(NC(=O)CN4CCOCC4)c3)s2)CC1] | N-[5-(4,4-dimethylpiperidin- 1-yl)-1,3,4-thiadiazol-2-yl]-3- [(morpholin-4-ylacetyl)- amino]-4-(trifluoromethoxy)- benzamide | 0.833 |

### Pharmaceutical Compositions of the Compounds of the Invention

This invention also relates to pharmaceutical compositions containing one or more compounds of the present invention. These compositions can be utilised to achieve the desired pharmacological effect by administration to a patient in need thereof. A patient, for the purpose of this invention, is a mammal, including a human, in need of treatment for the particular condition or disease. Therefore, the present invention includes pharmaceutical compositions that are comprised of a pharmaceutically acceptable carrier and a pharmaceutically effective amount of a compound, or salt thereof, of the present invention. A pharmaceutically acceptable carrier is preferably a carrier that is relatively non-toxic and innocuous to a patient at concentrations consistent with effective activity of the active ingredient so that any side effects ascribable to the carrier do not vitiate the beneficial effects of the active ingredient. A pharmaceutically effective amount of compound is preferably that amount which produces a result or exerts an influence on the particular condition being treated. The compounds of the present invention can be administered with pharmaceutically-acceptable carriers well known in the art using any effective conventional dosage unit forms, including immediate, slow and timed release preparations, orally, parenterally, topically, nasally, ophthalmically, optically, sublingually, rectally, vaginally, and the like.

For oral administration, the compounds can be formulated into solid or liquid preparations such as capsules, pills, tablets, troches, lozenges, melts, powders, solutions, suspensions, or emulsions, and may be prepared according to methods known to the art for the manufacture of pharmaceutical compositions. The solid unit dosage forms can be a capsule that can be of the ordinary hard- or soft-shelled gelatine type containing, for example, surfactants, lubricants, and inert fillers such as lactose, sucrose, calcium phosphate, and corn starch.

In another embodiment, the compounds of this invention may be tableted with conventional tablet bases such as lactose, sucrose and cornstarch in combination with binders such as acacia, corn starch or gelatine, disintegrating agents intended to assist the break-up and dissolution of the tablet following administration such as potato starch, alginic acid, corn starch, and guar gum, gum tragacanth, acacia, lubricants intended to improve the flow of tablet granulation and to prevent the adhesion of tablet material to the surfaces of the tablet dies and punches, for example talc, stearic acid, or magnesium, calcium or zinc stearate, dyes, colouring agents, and flavouring agents such as peppermint, oil of wintergreen, or cherry flavouring, intended to enhance the aesthetic qualities of the tablets and make them more acceptable to the patient. Suitable excipients for use in oral liquid dosage forms include dicalcium phosphate and diluents such as water and alcohols, for example, ethanol, benzyl alcohol, and polyethylene alcohols, either with or without the addition of a pharmaceutically acceptable surfactant, suspending agent or emulsifying agent. Various other materials may be present as coatings or to otherwise modify the physical form of the dosage unit. For instance tablets, pills or capsules may be coated with shellac, sugar or both.

Dispersible powders and granules are suitable for the preparation of an aqueous suspension. They provide the active ingredient in admixture with a dispersing or wetting agent, a suspending agent and one or more preservatives. Suitable dispersing or wetting agents and suspending agents are exemplified by those already mentioned above. Additional excipients, for example those sweetening, flavouring and colouring agents described above, may also be present.

The pharmaceutical compositions of this invention may also be in the form of oil-in-water emulsions. The oily phase may be a vegetable oil such as liquid paraffin or a mixture of vegetable oils. Suitable emulsifying agents may be (1) naturally occurring gums such as gum acacia and gum tragacanth, (2) naturally occurring phosphatides such as soy bean and lecithin, (3) esters or partial esters derived form fatty acids and hexitol anhydrides, for example, sorbitan monooleate, (4) condensation products of said partial esters with ethylene oxide, for example, polyoxyethylene sorbitan monooleate. The emulsions may also contain sweetening and flavouring agents.

Oily suspensions may be formulated by suspending the active ingredient in a vegetable oil such as, for example, arachis oil, olive oil, sesame oil or coconut oil, or in a mineral oil such as liquid paraffin. The oily suspensions may contain a thickening agent such as, for example, beeswax, hard paraffin, or cetyl alcohol. The suspensions may also contain one or more preservatives, for example, ethyl or n-propyl p-hydroxybenzoate ; one or more colouring agents ; one or more flavouring agents ; and one or more sweetening agents such as sucrose or saccharin.

Syrups and elixirs may be formulated with sweetening agents such as, for example, glycerol, propylene glycol, sorbitol or sucrose. Such formulations may also contain a demulcent, and preservative, such as methyl and propyl parabens and flavouring and colouring agents.

The compounds of this invention may also be administered parenterally, that is, subcutaneously, intravenously, intraocularly, intrasynovially, intramuscularly, or interperitoneally, as injectable dosages of the compound in preferably a physiologically acceptable diluent with a pharmaceutical carrier which can be a sterile liquid or mixture of liquids such as water, saline, aqueous dextrose and related sugar solutions, an alcohol such as ethanol, isopropanol, or hexadecyl alcohol, glycols such as propylene glycol or polyethylene glycol, glycerol ketals such as 2,2-dimethyl-1,1-dioxolane-4-methanol, ethers such as poly(ethylene glycol) 400, an oil, a fatty acid, a fatty acid ester or, a fatty acid glyceride, or an acetylated fatty acid glyceride, with or without the addition of a pharmaceutically acceptable surfactant such as a soap or a detergent, suspending agent such as pectin, carbomers, methylcellulose, hydroxypropylmethylcellulose, or carboxymethylcellulose, or emulsifying agent and other pharmaceutical adjuvants.

Illustrative of oils which can be used in the parenteral formulations of this invention are those of petroleum, animal, vegetable, or synthetic origin, for example, peanut oil, soybean oil, sesame oil, cottonseed oil, corn oil, olive oil, petrolatum and mineral oil. Suitable fatty acids include oleic acid, stearic acid, isostearic acid and myristic acid. Suitable fatty acid esters are, for example, ethyl oleate and isopropyl myristate. Suitable soaps include fatty acid alkali metal, ammonium, and triethanolamine salts and suitable detergents include cationic detergents, for example dimethyl dialkyl ammonium halides, alkyl pyridinium halides, and alkylamine acetates ; anionic detergents, for example, alkyl, aryl, and olefin sulfonates, alkyl, olefin, ether, and monoglyceride sulfates, and sulfosuccinates ; non-ionic detergents, for example, fatty amine oxides, fatty acid alkanolamides, and poly(oxyethylene-oxypropylene)s or ethylene oxide or propylene oxide copolymers ; and amphoteric detergents, for example, alkyl-beta-aminopropionates, and 2-alkylimidazoline quarternary ammonium salts, as well as mixtures.

The parenteral compositions of this invention will typically contain from about 0.5% to about 25% by weight of the active ingredient in solution. Preservatives and buffers may also be used advantageously. In order to minimise or eliminate irritation at the site of injection, such compositions may contain a non-ionic surfactant having a hydrophile-lipophile balance (HLB) preferably of from about 12 to about 17. The quantity of surfactant in such formulation preferably ranges from about 5% to about 15% by weight. The surfactant can be a single component having the above HLB or can be a mixture of two or more components having the desired HLB.

Illustrative of surfactants used in parenteral formulations are the class of polyethylene sorbitan fatty acid esters, for example, sorbitan monooleate and the high molecular weight adducts of ethylene oxide with a hydrophobic base, formed by the condensation of propylene oxide with propylene glycol.

The pharmaceutical compositions may be in the form of sterile injectable aqueous suspensions. Such suspensions may be formulated according to known methods using suitable dispersing or wetting agents and suspending agents such as, for example, sodium carboxymethylcellulose, methylcellulose, hydroxypropylmethyl-cellulose, sodium alginate, polyvinylpyrrolidone, gum tragacanth and gum acacia ; dispersing or wetting agents which may be a naturally occurring phosphatide such as lecithin, a condensation product of an alkylene oxide with a fatty acid, for example, polyoxyethylene stearate, a condensation product of ethylene oxide with a long chain aliphatic alcohol, for example, heptadeca-ethyleneoxycetanol, a condensation product of ethylene oxide with a partial ester derived form a fatty acid and a hexitol such as polyoxyethylene sorbitol monooleate, or a condensation product of an ethylene oxide with a partial ester derived from a fatty acid and a hexitol anhydride, for example polyoxyethylene sorbitan monooleate.

The sterile injectable preparation may also be a sterile injectable solution or suspension in a non-toxic parenterally acceptable diluent or solvent. Diluents and solvents that may be employed are, for example, water, Ringer's solution, isotonic sodium chloride solutions and isotonic glucose solutions. In addition, sterile fixed oils are conventionally employed as solvents or suspending media. For this purpose, any bland, fixed oil may be employed including synthetic mono- or diglycerides. In addition, fatty acids such as oleic acid can be used in the preparation of injectables.

A composition of the invention may also be administered in the form of suppositories for rectal administration of the drug. These compositions can be prepared by mixing the drug with a suitable non-irritation excipient which is solid at ordinary temperatures but liquid at the rectal temperature and will therefore melt in the rectum to release the drug. Such materials are, for example, cocoa butter and polyethylene glycol.

Another formulation employed in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art (see, e.g., U.S. Pat. No. 5,023,252, issued Jun. 11, 1991, incorporated herein by reference). Such patches may be constructed for continuous, pulsatile, or on demand delivery of pharmaceutical agents.

Controlled release formulations for parenteral administration include liposomal, polymeric microsphere and polymeric gel formulations that are known in the art.

It may be desirable or necessary to introduce the pharmaceutical composition to the patient via a mechanical delivery device. The construction and use of mechanical delivery devices for the delivery of pharmaceutical agents is well known in the art. Direct techniques for, for example, administering a drug directly to the brain usually involve placement of a drug delivery catheter into the patient's ventricular system to bypass the blood-brain barrier. One such implantable delivery system, used for the transport of agents to specific anatomical regions of the body, is described in U.S. Pat. No. 5,011,472, issued Apr. 30, 1991.

The compositions of the invention can also contain other conventional pharmaceutically acceptable compounding ingredients, generally referred to as carriers or diluents, as necessary or desired. Conventional procedures for preparing such compositions in appropriate dosage forms can be utilized.

Such ingredients and procedures include those described in the following references, each of which is incorporated herein by reference: Powell, M.F. et al., “Compendium of Excipients for Parenteral Formulations” PDA Journal of Pharmaceutical Science & Technology 1998, 52(5), 238-311 ; Strickley, R. G “Parenteral Formulations of Small Molecule Therapeutics Marketed in the United States (1999)-Part-1” PDA Journal of Pharmaceutical Science & Technology 1999, 53(6), 324-349 ; and Nema, S. et al., “Excipients and Their Use in Injectable Products” PDA Journal of Pharmaceutical Science & Technology 1997, 51(4), 166-171.

Commonly used pharmaceutical ingredients that can be used as appropriate to formulate the composition for its intended route of administration include:

acidifying agents (examples include but are not limited to acetic acid, citric acid, fumaric acid, hydrochloric acid, nitric acid);

alkalinizing agents (examples include but are not limited to ammonia solution, ammonium carbonate, diethanolamine, monoethanolamine, potassium hydroxide, sodium borate, sodium carbonate, sodium hydroxide, triethanolamine, trolamine);

adsorbents (examples include but are not limited to powdered cellulose and activated charcoal);

aerosol propellants (examples include but are not limited to carbon dioxide, CCI₂F₂, F₂CIC-CCIF₂ and CCIF₃)

air displacement agents (examples include but are not limited to nitrogen and argon);

antifungal preservatives (examples include but are not limited to benzoic acid, butylparaben, ethylparaben, methylparaben, propylparaben, sodium benzoate);

antimicrobial preservatives (examples include but are not limited to benzalkonium chloride, benzethonium chloride, benzyl alcohol, cetylpyridinium chloride, chlorobutanol, phenol, phenylethyl alcohol, phenylmercuric nitrate and thimerosal);

antioxidants (examples include but are not limited to ascorbic acid, ascorbyl palmitate, butylated hydroxyanisole, butylated hydroxytoluene, hypophosphorus acid, monothioglycerol, propyl gallate, sodium ascorbate, sodium bisulfite, sodium formaldehyde sulfoxylate, sodium metabisulfite);

binding materials (examples include but are not limited to block polymers, natural and synthetic rubber, polyacrylates, polyurethanes, silicones, polysiloxanes and styrene-butadiene copolymers);

buffering agents (examples include but are not limited to potassium metaphosphate, dipotassium phosphate, sodium acetate, sodium citrate anhydrous and sodium citrate dihydrate)

carrying agents (examples include but are not limited to acacia syrup, aromatic syrup, aromatic elixir, cherry syrup, cocoa syrup, orange syrup, syrup, corn oil, mineral oil, peanut oil, sesame oil, bacteriostatic sodium chloride injection and bacteriostatic water for injection)

chelating agents (examples include but are not limited to edetate disodium and edetic acid) colourants (examples include but are not limited to FD&C Red No. 3, FD&C Red No. 20, FD&C Yellow No. 6, FD&C Blue No. 2, D&C Green No. 5, D&C Orange No. 5, D&C Red No. 8, caramel and ferric oxide red);

clarifying agents (examples include but are not limited to bentonite);

emulsifying agents (examples include but are not limited to acacia, cetomacrogol, cetyl alcohol, glyceryl monostearate, lecithin, sorbitan monooleate, polyoxyethylene 50 monostearate);

encapsulating agents (examples include but are not limited to gelatin and cellulose acetate phthalate)

flavourants (examples include but are not limited to anise oil, cinnamon oil, cocoa, menthol, orange oil, peppermint oil and vanillin);

humectants (examples include but are not limited to glycerol, propylene glycol and sorbitol);

levigating agents (examples include but are not limited to mineral oil and glycerin);

oils (examples include but are not limited to arachis oil, mineral oil, olive oil, peanut oil, sesame oil and vegetable oil);

ointment bases (examples include but are not limited to lanolin, hydrophilic ointment, polyethylene glycol ointment, petrolatum, hydrophilic petrolatum, white ointment, yellow ointment, and rose water ointment);

penetration enhancers (transdermal delivery) (examples include but are not limited to monohydroxy or polyhydroxy alcohols, mono-or polyvalent alcohols, saturated or unsaturated fatty alcohols, saturated or unsaturated fatty esters, saturated or unsaturated dicarboxylic acids, essential oils, phosphatidyl derivatives, cephalin, terpenes, amides, ethers, ketones and ureas)

plasticizers (examples include but are not limited to diethyl phthalate and glycerol);

solvents (examples include but are not limited to ethanol, corn oil, cottonseed oil, glycerol, isopropanol, mineral oil, oleic acid, peanut oil, purified water, water for injection, sterile water for injection and sterile water for irrigation);

stiffening agents (examples include but are not limited to cetyl alcohol, cetyl esters wax, microcrystalline wax, paraffin, stearyl alcohol, white wax and yellow wax);

suppository bases (examples include but are not limited to cocoa butter and polyethylene glycols (mixtures));

surfactants (examples include but are not limited to benzalkonium chloride, nonoxynol 10, oxtoxynol 9, polysorbate 80, sodium lauryl sulfate and sorbitan mono-palmitate);

suspending agents (examples include but are not limited to agar, bentonite, carbomers, carboxymethylcellulose sodium, hydroxyethyl cellulose, hydroxypropyl cellulose, hydroxypropyl methylcellulose, kaolin, methylcellulose, tragacanth and veegum);

sweetening agents (examples include but are not limited to aspartame, dextrose, glycerol, mannitol, propylene glycol, saccharin sodium, sorbitol and sucrose);

tablet anti-adherents (examples include but are not limited to magnesium stearate and talc);

tablet binders (examples include but are not limited to acacia, alginic acid, carboxymethylcellulose sodium, compressible sugar, ethylcellulose, gelatin, liquid glucose, methylcellulose, non-crosslinked polyvinyl pyrrolidone, and pregelatinized starch);

tablet and capsule diluents (examples include but are not limited to dibasic calcium phosphate, kaolin, lactose, mannitol, microcrystalline cellulose, powdered cellulose, precipitated calcium carbonate, sodium carbonate, sodium phosphate, sorbitol and starch);

tablet coating agents (examples include but are not limited to liquid glucose, hydroxyethyl cellulose, hydroxypropyl cellulose, hydroxypropyl methylcellulose, methylcellulose, ethylcellulose, cellulose acetate phthalate and shellac);

tablet direct compression excipients (examples include but are not limited to dibasic calcium phosphate);

tablet disintegrants (examples include but are not limited to alginic acid, carboxymethylcellulose calcium, microcrystalline cellulose, polacrillin potassium, cross-linked polyvinylpyrrolidone, sodium alginate, sodium starch glycollate and starch);

tablet glidants (examples include but are not limited to colloidal silica, corn starch and talc);

tablet lubricants (examples include but are not limited to calcium stearate, magnesium stearate, mineral oil, stearic acid and zinc stearate);

tablet/capsule opaquants (examples include but are not limited to titanium dioxide);

tablet polishing agents (examples include but are not limited to carnuba wax and white wax);

thickening agents (examples include but are not limited to beeswax, cetyl alcohol and paraffin);

tonicity agents (examples include but are not limited to dextrose and sodium chloride);

viscosity increasing agents (examples include but are not limited to alginic acid, bentonite, carbomers, carboxymethylcellulose sodium, methylcellulose, polyvinyl pyrrolidone, sodium alginate and tragacanth); and

wetting agents (examples include but are not limited to heptadecaethylene oxycetanol, lecithins, sorbitol monooleate, polyoxyethylene sorbitol monooleate, and polyoxyethylene stearate).

Pharmaceutical compositions according to the present invention can be illustrated as follows:

Sterile IV Solution: A 5 mg/ml solution of the desired compound of this invention can be made using sterile, injectable water, and the pH is adjusted if necessary. The solution is diluted for administration to 1-2 mg/ml with sterile 5% dextrose and is administered as an IV infusion over about 60 minutes.

Lyophilised powder for IV administration: A sterile preparation can be prepared with (i) 100-1000 mg of the desired compound of this invention as a lyophilised powder, (ii) 32-327 mg/ml sodium citrate, and (iii) 300-3000 mg Dextran 40. The formulation is reconstituted with sterile, injectable saline or dextrose 5% to a concentration of 10 to 20 mg/ml, which is further diluted with saline or dextrose 5% to 0.2-0.4 mg/ml, and is administered either IV bolus or by IV infusion over 15-60 minutes.

Intramuscular suspension: The following solution or suspension can be prepared, for intramuscular injection:

50 mg/ml of the desired, water-insoluble compound of this invention

5 mg/ml sodium carboxymethylcellulose

4 mg/ml TWEEN 80

9 mg/ml sodium chloride

9 mg/ml benzyl alcohol

Hard Shell Capsules: A large number of unit capsules are prepared by filling standard two-piece hard galantine capsules each with 100 mg of powdered active ingredient, 150 mg of lactose, 50 mg of cellulose and 6 mg of magnesium stearate.

Soft Gelatin Capsules: A mixture of active ingredient in a digestible oil such as soybean oil, cottonseed oil or olive oil is prepared and injected by means of a positive displacement pump into molten gelatin to form soft gelatin capsules containing 100 mg of the active ingredient. The capsules are washed and dried. The active ingredient can be dissolved in a mixture of polyethylene glycol, glycerin and sorbitol to prepare a water miscible medicine mix.

Tablets: A large number of tablets are prepared by conventional procedures so that the dosage unit is 100 mg of active ingredient, 0.2 mg. of colloidal silicon dioxide, 5 mg of magnesium stearate, 275 mg of microcrystalline cellulose, 11 mg. of starch, and 98.8 mg of lactose. Appropriate aqueous and non-aqueous coatings may be applied to increase palatability, improve elegance and stability or delay absorption.

Immediate Release Tablets/Capsules: These are solid oral dosage forms made by conventional and novel processes. These units are taken orally without water for immediate dissolution and delivery of the medication. The active ingredient is mixed in a liquid containing ingredient such as sugar, gelatin, pectin and sweeteners. These liquids are solidified into solid tablets or caplets by freeze drying and solid state extraction techniques. The drug compounds may be compressed with viscoelastic and thermoelastic sugars and polymers or effervescent components to produce porous matrices intended for immediate release, without the need of water.

Methods of Treatment

The compounds and compositions provided herein can be used as inhibitors of one or more members of the Wnt pathway, including one or more Wnt proteins, and thus can be used to treat a variety of disorders and diseases in which aberrant Wnt signaling is implicated, such as cancer and other diseases associated with abnormal angiogenesis, cellular proliferation, and cell cycling. Accordingly, the compounds and compositions provided herein can be used to treat cancer, to reduce or inhibit angiogenesis, to reduce or inhibit cellular proliferation and correct a genetic disorder due to mutations in Wnt signaling components. Non-limiting examples of diseases which can be treated with the compounds and compositions provided herein include a variety of cancers, diabetic retinopathy, neovascular glaucoma, rheumatoid arthritis, psoriasis, mycotic and viral infections, osteochondrodysplasia, Alzheimer's disease, osteoarthritis, polyposis coli, osteoporosis-pseudoglioma syndrome, familial exudative vitreoretinopathy, retinal angiogenesis, early coronary disease, tetra-amelia syndrome, Müllerian-duct regression and virilization, SERKAL syndrome, diabetes mellitus type 2, Fuhrmann syndrome, Al-Awadi/Raas-Rothschild/Schinzel phocomelia syndrome, odonto-onycho-dermal dysplasia, obesity, split-hand/foot malformation, caudal duplication syndrome, tooth agenesis, Wilms tumor, skeletal dysplasia, focal dermal hypoplasia, autosomal recessive anonychia, neural tube defects, alpha-thalassemia (ATRX) syndrome, fragile X syndrome, ICF syndrome, Angelman syndrome, Prader-Willi syndrome, Beckwith-Wiedemarm Syndrome and Rett syndrome.

In accordance with another aspect therefore, the present invention covers a compound of general formula (I), or a stereoisomer, a tautomer, an N-oxide, a hydrate, a solvate, or a salt thereof, particularly a pharmaceutically acceptable salt thereof, or a mixture of same, as described and defined herein, for use in the treatment or prophylaxis of a disease, as mentioned supra.

Another particular aspect of the present invention is therefore the use of a compound of general formula (I), described supra, or a stereoisomer, a tautomer, an N-oxide, a hydrate, a solvate, or a salt thereof, particularly a pharmaceutically acceptable salt thereof, or a mixture of same, for the prophylaxis or treatment of a disease.

Another particular aspect of the present invention is therefore the use of a compound of general formula (I) described supra for manufacturing a pharmaceutical composition for the treatment or prophylaxis of a disease.

The term “pharmaceutically acceptable salt” refers to a relatively non-toxic, inorganic or organic acid addition salt of a compound of the present invention. For example, see S. M. Berge, et al. “Pharmaceutical Salts,” J. Pharm. Sci. 1977, 66, 1-19.

A suitable pharmaceutically acceptable salt of the compounds of the present invention may be, for example, an acid-addition salt of a compound of the present invention bearing a nitrogen atom, in a chain or in a ring, for example, which is sufficiently basic, such as an acid-addition salt with an inorganic acid, such as hydrochloric, hydrobromic, hydroiodic, sulfuric, bisulfuric, phosphoric, or nitric acid, for example, or with an organic acid, such as formic, acetic, acetoacetic, pyruvic, trifluoroacetic, propionic, butyric, hexanoic, heptanoic, undecanoic, lauric, benzoic, salicylic, 2-(4-hydroxybenzoyl)-benzoic, camphoric, cinnamic, cyclopentanepropionic, digluconic, 3-hydroxy-2-naphthoic, nicotinic, pamoic, pectinic, persulfuric, 3-phenylpropionic, picric, pivalic, 2-hydroxyethanesulfonate, itaconic, sulfamic, trifluoromethanesulfonic, dodecylsulfuric, ethansulfonic, benzenesulfonic, para-toluenesulfonic, methansulfonic, 2-naphthalenesulfonic, naphthalinedisulfonic, camphorsulfonic acid, citric, tartaric, stearic, lactic, oxalic, malonic, succinic, malic, adipic, alginic, maleic, fumaric, D-gluconic, mandelic, ascorbic, glucoheptanoic, glycerophosphoric, aspartic, sulfosalicylic, hemisulfuric, or thiocyanic acid, for example.

Further, another suitably pharmaceutically acceptable salt of a compound of the present invention which is sufficiently acidic, is an alkali metal salt, for example a sodium or potassium salt, an alkaline earth metal salt, for example a calcium or magnesium salt, an ammonium salt or a salt with an organic base which affords a physiologically acceptable cation, for example a salt with N-methyl-glucamine, dimethyl-glucamine, ethyl-glucamine, lysine, dicyclohexylamine, 1,6-hexadiamine, ethanolamine, glucosamine, sarcosine, serinol, tris-hydroxy-methyl-aminomethane, aminopropandiol, sovak-base, 1-amino-2,3,4-butantriol. Additionally, basic nitrogen containing groups may be quaternised with such agents as lower alkyl halides such as methyl, ethyl, propyl, and butyl chlorides, bromides and iodides; dialkyl sulfates like dimethyl, diethyl, and dibutyl sulfate; and diamyl sulfates, long chain halides such as decyl, lauryl, myristyl and strearyl chlorides, bromides and iodides, aralkyl halides like benzyl and phenethyl bromides and others.

Those skilled in the art will further recognise that acid addition salts of the claimed compounds may be prepared by reaction of the compounds with the appropriate inorganic or organic acid via any of a number of known methods. Alternatively, alkali and alkaline earth metal salts of acidic compounds of the invention are prepared by reacting the compounds of the invention with the appropriate base via a variety of known methods.

Method of Treating Hyper-proliferative Disorders

The present invention relates to a method for using the compounds of the present invention and compositions thereof, to treat mammalian hyper-proliferative disorders. Compounds can be utilized to inhibit, block, reduce, decrease, etc., cell proliferation and/or cell division, and/or produce apoptosis. This method comprises administering to a mammal in need thereof, including a human, an amount of a compound of this invention, or a pharmaceutically acceptable salt, isomer, polymorph, metabolite, hydrate, solvate or ester thereof ; etc. which is effective to treat the disorder.

Hyper-proliferative disorders include but are not limited, e.g., psoriasis, keloids, and other hyperplasias affecting the skin, benign prostate hyperplasia (BPH), solid tumours, such as cancers of the breast, respiratory tract, brain, reproductive organs, digestive tract, urinary tract, eye, liver, skin, head and neck, thyroid, parathyroid and their distant metastases. Those disorders also include lymphomas, sarcomas, and leukaemias.

Examples of breast cancer include, but are not limited to invasive ductal carcinoma, invasive lobular carcinoma, ductal carcinoma in situ, and lobular carcinoma in situ.

Examples of cancers of the respiratory tract include, but are not limited to small-cell and non-small-cell lung carcinoma, as well as bronchial adenoma and pleuropulmonary blastoma.

Examples of brain cancers include, but are not limited to brain stem and hypophtalmic glioma, cerebellar and cerebral astrocytoma, medulloblastoma, ependymoma, as well as neuroectodermal and pineal tumour.

Tumours of the male reproductive organs include, but are not limited to prostate and testicular cancer. Tumours of the female reproductive organs include, but are not limited to endometrial, cervical, ovarian, vaginal, and vulvar cancer, as well as sarcoma of the uterus.

Tumours of the digestive tract include, but are not limited to anal, colon, colorectal, oesophageal, gallbladder, gastric, pancreatic, rectal, small-intestine, and salivary gland cancers.

Tumours of the urinary tract include, but are not limited to bladder, penile, kidney, renal pelvis, ureter, urethral and human papillary renal cancers.

Eye cancers include, but are not limited to intraocular melanoma and retinoblastoma.

Examples of liver cancers include, but are not limited to hepatocellular carcinoma (liver cell carcinomas with or without fibrolamellar variant), cholangiocarcinoma (intrahepatic bile duct carcinoma), and mixed hepatocellular cholangiocarcinoma.

Skin cancers include, but are not limited to squamous cell carcinoma, Kaposi's sarcoma, malignant melanoma, Merkel cell skin cancer, and non-melanoma skin cancer.

Head-and-neck cancers include, but are not limited to laryngeal, hypopharyngeal, nasopharyngeal, oropharyngeal cancer, lip and oral cavity cancer and squamous cell. Lymphomas include, but are not limited to AIDS-related lymphoma, non-Hodgkin's lymphoma, cutaneous T-cell lymphoma, Burkitt lymphoma, Hodgkin's disease, and lymphoma of the central nervous system.

Sarcomas include, but are not limited to sarcoma of the soft tissue, osteosarcoma, malignant fibrous histiocytoma, lymphosarcoma, and rhabdomyosarcoma.

Leukemias include, but are not limited to acute myeloid leukemia, acute lymphoblastic leukemia, chronic lymphocytic leukemia, chronic myelogenous leukemia, and hairy cell leukemia.

These disorders have been well characterized in humans, but also exist with a similar etiology in other mammals, and can be treated by administering pharmaceutical compositions of the present invention.

The term “treating” or “treatment” as stated throughout this document is used conventionally, e.g., the management or care of a subject for the purpose of combating, alleviating, reducing, relieving, improving the condition of, etc., of a disease or disorder, such as a carcinoma.

Dose and Administration

Based upon standard laboratory techniques known to evaluate compounds useful for the treatment of hyper-proliferative disorders and angiogenic disorders, by standard toxicity tests and by standard pharmacological assays for the determination of treatment of the conditions identified above in mammals, and by comparison of these results with the results of known medicaments that are used to treat these conditions, the effective dosage of the compounds of this invention can readily be determined for treatment of each desired indication. The amount of the active ingredient to be administered in the treatment of one of these conditions can vary widely according to such considerations as the particular compound and dosage unit employed, the mode of administration, the period of treatment, the age and sex of the patient treated, and the nature and extent of the condition treated.

The total amount of the active ingredient to be administered will generally range from about 0.001 mg/kg to about 200 mg/kg body weight per day, and preferably from about 0.01 mg/kg to about 20 mg/kg body weight per day. Clinically useful dosing schedules will range from one to three times a day dosing to once every four weeks dosing. In addition, “drug holidays” in which a patient is not dosed with a drug for a certain period of time, may be beneficial to the overall balance between pharmacological effect and tolerability. A unit dosage may contain from about 0.5 mg to about 1500 mg of active ingredient, and can be administered one or more times per day or less than once a day. The average daily dosage for administration by injection, including intravenous, intramuscular, subcutaneous and parenteral injections, and use of infusion techniques will preferably be from 0.01 to 200 mg/kg of total body weight. The average daily rectal dosage regimen will preferably be from 0.01 to 200 mg/kg of total body weight. The average daily vaginal dosage regimen will preferably be from 0.01 to 200 mg/kg of total body weight. The average daily topical dosage regimen will preferably be from 0.1 to 200 mg administered between one to four times daily. The transdermal concentration will preferably be that required to maintain a daily dose of from 0.01 to 200 mg/kg. The average daily inhalation dosage regimen will preferably be from 0.01 to 100 mg/kg of total body weight.

Of course the specific initial and continuing dosage regimen for each patient will vary according to the nature and severity of the condition as determined by the attending diagnostician, the activity of the specific compound employed, the age and general condition of the patient, time of administration, route of administration, rate of excretion of the drug, drug combinations, and the like. The desired mode of treatment and number of doses of a compound of the present invention or a pharmaceutically acceptable salt or ester or composition thereof can be ascertained by those skilled in the art using conventional treatment tests.

Preferably, the diseases of said method are haematological tumours, solid tumour and/or metastases thereof.

The compounds of the present invention can be used in particular in therapy and prevention, i.e. prophylaxis, of tumour growth and metastases, especially in solid tumours of all indications and stages with or without pre-treatment of the tumour growth.

Methods of testing for a particular pharmacological or pharmaceutical property are well known to persons skilled in the art.

The example testing experiments described herein serve to illustrate the present invention and the invention is not limited to the examples given.

Combination Therapies

The term “combination” in the present invention is used as known to persons skilled in the art and may be present as a fixed combination, a non-fixed combination or kit-of-parts.

A “fixed combination” in the present invention is used as known to persons skilled in the art and is defined as a combination wherein the said first active ingredient and the said second active ingredient are present together in one unit dosage or in a single entity. One example of a “fixed combination” is a pharmaceutical composition wherein the said first active ingredient and the said second active ingredient are present in admixture for simultaneous administration, such as in a formulation. Another example of a “fixed combination” is a pharmaceutical combination wherein the said first active ingredient and the said second active ingredient are present in one unit without being in admixture.

A non-fixed combination or “kit-of-parts” in the present invention is used as known to persons skilled in the art and is defined as a combination wherein the said first active ingredient and the said second active ingredient are present in more than one unit. One example of a non-fixed combination or kit-of-parts is a combination wherein the said first active ingredient and the said second active ingredient are present separately. The components of the non-fixed combination or kit-of-parts may be administered separately, sequentially, simultaneously, concurrently or chronologically staggered.

The compounds of this invention can be administered as the sole pharmaceutical agent or in combination with one or more other pharmaceutical agents where the combination causes no unacceptable adverse effects. The present invention relates also to such combinations. For example, the compounds of this invention can be combined with known chemotherapeutic agents or anti-cancer agents, e.g. anti-hyper-proliferative or other indication agents, and the like, as well as with admixtures and combinations thereof. Other indication agents include, but are not limited to, anti-angiogenic agents, mitotic inhibitors, alkylating agents, anti-metabolites, DNA-intercalating antibiotics, growth factor inhibitors, cell cycle inhibitors, enzyme inhibitors, toposisomerase inhibitors, biological response modifiers, or anti-hormones.

The term “(chemotherapeutic) anti-cancer agents”, includes but is not limited to 1311-chTNT, abarelix, abiraterone, aclarubicin, aldesleukin, alemtuzumab, alitretinoin, altretamine, aminoglutethimide, amrubicin, amsacrine, anastrozole, arglabin, arsenic trioxide, asparaginase, azacitidine, basiliximab, BAY 80-6946, BAY 1000394, belotecan, bendamustine, bevacizumab, bexarotene, bicalutamide, bisantrene, bleomycin, bortezomib, buserelin, busulfan, cabazitaxel, calcium folinate, calcium levofolinate, capecitabine, carboplatin, carmofur, carmustine, catumaxomab, celecoxib, celmoleukin, cetuximab, chlorambucil, chlormadinone, chlormethine, cisplatin, cladribine, clodronic acid, clofarabine, crisantaspase, cyclophosphamide, cyproterone, cytarabine, dacarbazine, dactinomycin, darbepoetin alfa, dasatinib, daunorubicin, decitabine, degarelix, denileukin diftitox, denosumab, deslorelin, dibrospidium chloride, docetaxel, doxifluridine, doxorubicin, doxorubicin+estrone, eculizumab, edrecolomab, elliptinium acetate, eltrombopag, endostatin, enocitabine, epirubicin, epitiostanol, epoetin alfa, epoetin beta, eptaplatin, eribulin, erlotinib, estradiol, estramustine, etoposide, everolimus, exemestane, fadrozole, filgrastim, fludarabine, fluorouracil, flutamide, formestane, fotemustine, fulvestrant, gallium nitrate, ganirelix, gefitinib, gemcitabine, gemtuzumab, glutoxim, goserelin, histamine dihydrochloride, histrelin, hydroxycarbamide, 1-125 seeds, ibandronic acid, ibritumomab tiuxetan, idarubicin, ifosfamide, imatinib, imiquimod, improsulfan, interferon alfa, interferon beta, interferon gamma, ipilimumab, irinotecan, ixabepilone, lanreotide, lapatinib, lenalidomide, lenograstim, lentinan, letrozole, leuprorelin, levamisole, lisuride, lobaplatin, lomustine, lonidamine, masoprocol, medroxyprogesterone, megestrol, melphalan, mepitiostane, mercaptopurine, methotrexate, methoxsalen, Methyl aminolevulinate, methyltestosterone, mifamurtide, miltefosine, miriplatin, mitobronitol, mitoguazone, mitolactol, mitomycin, mitotane, mitoxantrone, nedaplatin, nelarabine, nilotinib, nilutamide, nimotuzumab, nimustine, nitracrine, ofatumumab, omeprazole, oprelvekin, oxaliplatin, p53 gene therapy, paclitaxel, palifermin, palladium-103 seed, pamidronic acid, panitumumab, pazopanib, pegaspargase, PEG-epoetin beta (methoxy PEG-epoetin beta), pegfilgrastim, peginterferon alfa-2b, pemetrexed, pentazocine, pentostatin, peplomycin, perfosfamide, picibanil, pirarubicin, plerixafor, plicamycin, poliglusam, polyestradiol phosphate, polysaccharide-K, porfimer sodium, pralatrexate, prednimustine, procarbazine, quinagolide, radium-223 chloride, raloxifene, raltitrexed, ranimustine, razoxane, refametinib, regorafenib, risedronic acid, rituximab, romidepsin, romiplostim, sargramostim, sipuleucel-T, sizofiran, sobuzoxane, sodium glycididazole, sorafenib, streptozocin, sunitinib, talaporfin, tamibarotene, tamoxifen, tasonermin, teceleukin, tegafur, tegafur+gimeracil+oteracil, temoporfin, temozolomide, temsirolimus, teniposide, testosterone, tetrofosmin, thalidomide, thiotepa, thymalfasin, tioguanine, tocilizumab, topotecan, toremifene, tositumomab, trabectedin, trastuzumab, treosulfan, tretinoin, trilostane, triptorelin, trofosfamide, tryptophan, ubenimex, valrubicin, vandetanib, vapreotide, vemurafenib, vinblastine, vincristine, vindesine, vinflunine, vinorelbine, vorinostat, vorozole, yttrium-90 glass microspheres, zinostatin, zinostatin stimalamer, zoledronic acid, zorubicin.

Generally, the use of cytotoxic and/or cytostatic agents in combination with a compound or composition of the present invention will serve to:
(1) yield better efficacy in reducing the growth of a tumor or even eliminate the tumor as compared to administration of either agent alone,
(2) provide for the administration of lesser amounts of the administered chemotherapeutic agents,
(3) provide for a chemotherapeutic treatment that is well tolerated in the patient with fewer deleterious pharmacological complications than observed with single agent chemotherapies and certain other combined therapies,
(4) provide for treating a broader spectrum of different cancer types in mammals, especially humans,
(5) provide for a higher response rate among treated patients,
(6) provide for a longer survival time among treated patients compared to standard chemotherapy treatments,
(7) provide a longer time for tumor progression, and/or
(8) yield efficacy and tolerability results at least as good as those of the agents used alone, compared to known instances where other cancer agent combinations produce antagonistic effects.

Biological Assays

Examples were tested in selected biological assays one or more times. When tested more than once, data are reported as either average values or as median values, wherein
the average value, also referred to as the arithmetic mean value, represents the sum of the values obtained divided by the number of times tested, and
the median value represents the middle number of the group of values when ranked in ascending or descending order. If the number of values in the data set is odd, the median is the middle value. If the number of values in the data set is even, the median is the arithmetic mean of the two middle values.

Examples were synthesized one or more times. When synthesized more than once, data from biological assays represent average values or median values calculated utilizing data sets obtained from testing of one or more synthetic batch.

Some of the compounds of general formula (I) show low solubility in aqueous media and organic solvents. This can affect the possibility to assess the activity of such compounds with the described assays. Therefore, the high ICₛo value of some compound might be a result of the low solubility.

Measurement of the Inhibitory Activity of Selected Compounds on the Wnt Signaling Cascade

In order to discover and characterize small molecules which inhibit the constitutive active colorectal cancer cell (CRC) Wnt pathway, a cellular reporter assay was employed. The corresponding assay cell was generated by transfection of the colorectal cancer cell line HCT116 (ATCC, #CCL-247) with the Super TopFlash vector (Morin, Science 275, 1997, 1787-1790; Molenaar et al., Cell 86 (3), 1996, 391-399). The HCT116 cell line is cultivated at 37° C. and 5% CO₂ in DMEM/F-12 (Life Technologies, #11320-074), supplemented with 2 mM glutamine, 20 mM HEPES, 1.4 mM pyruvate, 0.15% Na-bicarbonate and 10% foetal bovine serum (GIBCO, #10270), this cancer cell line is pathophysiological relevant since it carries a deletion of position S45 in the β-catenin gene, leading to constitutive active Wnt signaling. Stable transfectants were generated by cotransfection with pcDNA3 and selection of stable transfected cells with 1 mg/ml G418.

In a parallel approach, HCT116 cells were cotransfected with the FOP control vector and pcDNA3. The FOP vector is identical to the TOP construct, but it contains instead of functional TCF elements a randomized, non-functional sequence. For this transfection a stable transfected cell line was generated as well.

In preparation of the assay, the two cell lines were plated 24 hours before at 10000 cells per well of a 384 micro titre plate (MTP) in 30 μL growth medium. Selective inhibitory activity for small molecules on the mutated Wnt pathway was determined after parallel incubation of both (TOP and FOP) HCT116 reporter cell lines with a compound dilution series from 50 μM to 15 nM in steps of 3.16-fold dilutions in CAFTY buffer (130 mM NaCI, 5 mM KCI, 20 mM HEPES, 1 mM MgCl₂, 5 mM NaHCO₃, pH 7.4) containing 2 mM Ca²⁺ and 0.01% BSA. The compounds were thereby serially prediluted in 100% DMSO and thereafter in addition 50 fold into the CAFTY compound dilution buffer (described above). From this dilution 10 μL were added to the cells in 30 μL growth medium and incubated for 36 hours at 37° C. and 5% CO₂. Thereafter luciferase assay buffer (1:1 mixture of luciferase substrate buffer (20 mM Tricine, 2.67 mM MgSO₄, 0.1 mM EDTA, 4 mM DTT, 270 μM Coenzyme A, 470 μM Luciferin, 530 μM ATP, ph adjusted to pH 7.8 with a sufficient volume of 5M NaOH) and Triton buffer (30 mL Triton X-100, 115 mL glycerol, 308 mg Dithiothreitol, 4.45 g Na₂HPO₄. 2 H₂O, 3.03 g TRIS HCl, ad 1I H₂O, pH 7.8) was added as equal volume to the compound solution on the cells to determine luciferase expression as a measure of Wnt signaling activity in a luminometer.

In order to determine the inhibitory activity of compounds for the WT Wnt signaling pathway, the Super TopFlash vector respectively FOP vector were cotransfected with pcDNA3 into HEK293 and stable transfected HEK293 cells were isolated by antibiotic selection. In preparation of compound testing, a dose response curve for the Wnt dependent luciferase expression was recorded by stimulating the assay cells with human recombinant Wnt-3a (R&D, #5036-WN-010) at different concentrations for 16 hours at 37° C. and 5% CO₂ followed by subsequent luciferase measurement as described above to determine the Wnt-3a EC50 for the HEK293 TOP cell line on the day of testing.

The recombinant human Wnt-3a was thereby used between 2500 and 5 ng/ml in two-fold dilution steps. To determine the inhibitory activity of compounds on the WT Wnt pathway they were prepared and diluted as described above for the constitutive active Wnt pathway and coincubated with the ECₛo concentration of Wnt-3a for 16 hours at 37° C. and 5% CO₂ on the HEK293 TOP respectively control HEK293 FOP cells. Measurement of luciferase expression was done as described for the constitutive active Wnt assay.

TABLE 2

| | HCT116-TOP | HCT116-FOP |
|---|---|---|
| | IC₅₀ [M] | IC₅₀ [M] |
| Example No | [mol/L] | mol/L] |
| 1 | 3.60E−7 | ≧5.00E−5 |
| 2 | 4.15E−8 | ≧5.00E−5 |
| 3 | 8.60E−8 | ≧5.00E−5 |
| 4 | 4.92E−7 | ≧5.00E−5 |
| 5 | 2.70E−7 | ≧5.00E−5 |
| 6 | 6.55E−8 | 4.65E−5 |
| 7 | 1.10E−7 | ≧5.00E−5 |
| 8 | 6.55E−7 | ≧5.00E−5 |
| 9 | 8.30E−7 | 1.80E−5 |
| 10 | 8.40E−7 | ≧5.00E−5 |
| 11 | 2.20E−7 | ≧5.00E−5 |
| 12 | 2.60E−7 | 1.50E−5 |
| 13 | 1.40E−6 | ≧5.00E−5 |
| 14 | 1.45E−6 | ≧5.00E−5 |
| 15 | 1.99E−6 | ≧5.00E−5 |
| 16 | 2.10E−6 | ≧5.00E−5 |
| 17 | 2.85E−6 | 4.10E−5 |
| 18 | 3.15E−6 | ≧5.00E−5 |
| 19 | 3.80E−6 | ≧5.00E−5 |
| 20 | 3.91E−6 | ≧5.00E−5 |
| 21 | 4.10E−6 | ≧5.00E−5 |
| 22 | 4.30E−6 | ≧5.00E−5 |
| 23 | 4.59E−6 | ≧5.00E−5 |
| 24 | 4.60E−6 | ≧5.00E−5 |
| 25 | 1.30E−5 | ≧5.00E−5 |
| 26 | 1.50E−5 | ≧5.00E−5 |
| 27 | 2.00E−5 | ≧5.00E−5 |
| 28 | 2.00E−5 | 2.69E−5 |
| 29 | 2.68E−5 | ≧5.00E−5 |
| 30 | 3.35E−5 | ≧5.00E−5 |
| 31 | 3.41E−5 | ≧5.00E−5 |
| 32 | 5.00E−5 | ≧5.00E−5 |
| 33 | 5.00E−5 | ≧5.00E−5 |
| 34 | 5.00E−5 | ≧5.00E−5 |

Measurement of the Inhibitory Activity of Selected Compounds on the Wildtype Wnt Signaling Cascade

In order to discover and characterize small molecules which inhibit the wildtype Wnt pathway, a cellular reporter assay was employed. The corresponding assay cell was generated by transfection of the mammalian cell line HEK293 (ATCC, #CRL-1573) with the Super TopFlash vector (Morin, Science 275, 1997, 1787-1790; Molenaar et al., Cell 86 (3), 1996, 391-399). The HEK293 cell line is cultivated at 37° C. and 5% CO₂ in DMEM (Life Technologies, #41965-039), supplemented with 2 mM glutamine, 20 mM HEPES, 1.4 mM pyruvate, 0.15% Na-bicarbonate and 10% foetal bovine serum (GIBCO, #10270). Stable transfectants were generated by selection with 300 μg/ml Hygromycin.

In a parallel approach, HEK293 cells were cotransfected with the FOP control vector and pcDNA3. The FOP vector is identical to the TOP construct, but it contains instead of functional TCF elements a randomized, non-functional sequence. For this transfection a stable transfected cell line was generated as well, based on selection with Geneticin (1 mg/ml). In preparation of the assay, the two cell lines were plated 24 hours before beginning the test at 10000 cells per well in a 384 micro titre plate (MTP) in 30 μl growth medium. Before compound testing a dose response curve for the Wnt dependent luciferase expression was recorded by stimulating the assay cell line with human recombinant Wnt-3a (R&D, #5036-WN-010) at different concentrations for 16 hours at 37° C. and 5% CO₂ followed by subsequent luciferase measurement, to determine the Wnt-3a ECₛo for the HEK293 TOP cell line on the day of testing. The recombinant human Wnt-3a was thereby applied between 2500 and 5 ng/ml in two-fold dilution steps. Selective inhibitory activity for small molecules on the wildtype Wnt pathway was determined after parallel incubation of both (TOP and FOP) HEK293 reporter cell lines with a compound dilution series from 50 μM to 15 nM in steps of 3.16-fold dilutions in CAFTY buffer (130 mM NaCI, 5 mM KCI, 20 mM HEPES, 1 mM MgCl₂, 5 mM NaHCO₃, pH 7.4) containing 2 mM Ca²⁺ and 0.01% BSA.

The compounds were thereby serially prediluted in 100% DMSO and thereafter 50 fold into the CAFTY compound dilution buffer (described above). From this dilution 10 μl were added in combination with the EC₅₀ concentration of recombinant Wnt3a to the cells in 30 μl growth medium and incubated for 16 hours at 37° C. and 5% CO₂. Thereafter luciferase assay buffer (1:1 mixture of luciferase substrate buffer (20 mM Tricine, 2.67 mM MgSO₄, 0.1 mM EDTA, 4 mM DTT, 270 μM Coenzyme A, 470 μM Luciferin, 530 μM ATP, ph adjusted to pH 7.8 with a sufficient volume of 5M NaOH) and Triton buffer (30 ml Triton X-100, 115 ml glycerol, 308 mg Dithiothreitol, 4.45 g Na₂HPO₄ 2 H₂O, 3.03 g TRIS HCI (CAS Number 1185-53-1), ad 11 H₂O, pH 7.8) was added in an equal volume to determine luciferase expression as a measure of Wnt signaling activity in a luminometer. The Wnt inhibitory activity was determined as ICₛo of resulting dose response curves.

QPCR Protocol

Real-time RT-PCR using a TaqMan fluorogenic detection system is a simple and sensitive assay for quantitative analysis of gene transcription. The TaqMan fluorogenic detection system can monitor PCR in real time using a dual-labeled fluorogenic hybridization probe (TaqMan probe) and a polymerase with 5′-3′ exonuclease activity.

Cells from different cancer cell lines (as HCT116, but not limited to) were grown at 500-1000 cells/well in 384 well cell culture plates. For cell lysis the cell medium was carefully removed. The cells were washed carefully once with 50 μL/well PBS. Then 9.75 μL/well cell lysis buffer (50 mM TRIS HCI pH 8,0, 40 mM NaCI, 1,5 mM MgCl₂, 0,5% IGEPAL CA 630, 50 mM Guanidium thiocyanate) and 0.25 μL RNASeOUT (40 U/ul, Invitrogen, 10777-019)) per well were added. The plate was incubated for 5 min at room temperature. Then 30 μL DNAse/RNAse-free water per well added and the lysates were mixed. For the One-Step RT-PCR 2 μL lysate (each) was transferred to a 384 well PCR plate. The PCR reaction was composed by 5 μL 2× One Step RT qPCR MasterMix Plus, 0.05 μL Euroscript RT/RNAse Inhibitor (50 U/μl, 20 U/μl) and 200 nM of the appropriate Primer/Hydrolysis Probe mix (primer sequences of forward, reverse and probe are given below for each analysed gene of interest or house keeping gene). 10 μL water were added per well. Seal the plate with an adhesive optical film. The RT-PCR protocol was setup with 30 min 48° C., then 10 min 95° C. followed by 50 cycles of 15 sec 95° C./1 min 60° C. and a cooling step of 40° C. for 30 sec using a Lightcycler L5440 from Roche. Relative expression was calculated using CP values from the gene of interest (e.g. AXIN2, but not limited to) and a house keeping gene (L32).

Used Primers

L32
(forward primer: AAGTTCATCCGGCACCAGTC;
reverse primer: TGGCCCTTGAATCTTCTACGA;
probe: CCCAGAGGCATTGACAACAGGG)
AXIN2
(forward primer: AGGCCAGTGAGTTGGTTGTC;
reverse primer: AGCTCTGAGCCTTCAGCATC;
probe: TCTGTGGGGAAGAAATTCCATACCG)

Sequence Listings

SEQ ID NO

| | |
|---|---|
| 1 | AAGTTCATCCGGCACCAGTC |
| 2 | TGGCCCTTGAATCTTCTACGA |
| 3 | CCCAGAGGCATTGACAACAGGG |
| 4 | AGGCCAGTGAGTTGGTTGTC |
| 5 | AGCTCTGAGCCTTCAGCATC |
| 6 | TCTGTGGGGAAGAAATTCCATACCG |

1. A compound of formula (I): 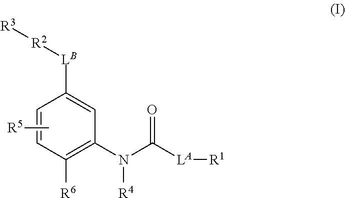
wherein:
L^{A} represents is
*CH₂**;
wherein * indicates the point of attachment to the carbonyl group, and ** indicates the point of attachment to R¹;
L^{B} is *N(H)—C(═O)**;
wherein * indicates the point of attachment to R², and ** indicates the point of attachment to the phenyl group;
R¹ is a group selected from: 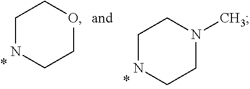
wherein * indicates the point of attachment to L^{A},
R² 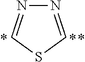
wherein * indicates the point of attachment to R³, and ** indicates the point of attachment to L^{B};
R³ is a group selected from: 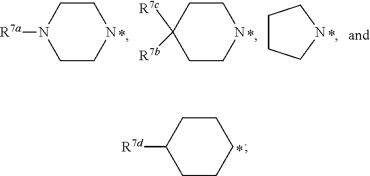
wherein * indicates the point of attachment to R²;
R⁴ is a hydrogen atom;
R⁵ is a hydrogen atom;
R⁶ is a —O—CF₃ group;
R^{7a} is a group selected from:
—C(═O)—R⁸, —C(═O)—O—R⁸, —C(═O)—N(R⁸)(R⁹), and —S(═O)₂—N(R⁸)(R⁹),
R^{7b} represents is a hydrogen atom or a methyl-group;
R^{7c} is a hydrogen atom or a group selected from:
methyl-, —OH, HO—(C₁-C₃-alkyl)-, methoxy-, and —C(═O)—O—R⁸;
R^{7d} is a hydrogen atom;
R⁸ is a group selected from:
—CH₃, —CH₂-CH₃, —C(H)(CH₃)₂, —C(CH₃)₃, and -cyclopropyl;
R⁹ is a -CH₃ group;
or a tautomer, an N-oxide, a hydrate, a solvate, or a salt thereof, or a mixture of any of the foregoing.

2. The compound according to claim 1, or a tautomer, an N-oxide, a hydrate, a solvate, or a salt thereof, or a mixture of any of the foregoing, wherein:
R¹ is

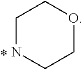

3. The compound according to claim 1, or a tautomer, an N-oxide, a hydrate, a solvate, or a salt thereof, or a mixture of any of the foregoing, wherein :
R¹ is

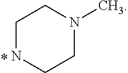

4. The compound according to claim 1, or a tautomer, an N-oxide, a hydrate, a solvate, or a salt thereof, or a mixture of any of the foregoing, wherein:
R³ is selected from: 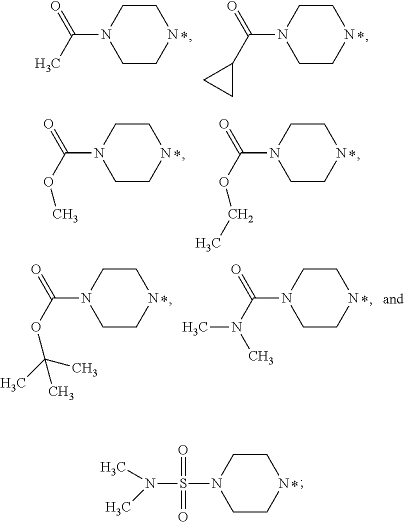
wherein * indicates the point of attachment to R².

5. The compound according to claim 1, or a tautomer, an N-oxide, a hydrate, a solvate, or a salt thereof, or a mixture of any of the foregoing, wherein:
R³ is selected from: 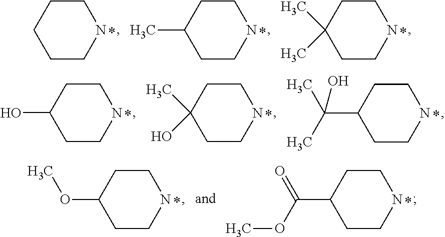
wherein * indicates the point of attachment to R².

6. (canceled)

7. The compound according to claim 1, or a tautomer, an N-oxide, a hydrate, a solvate, or a salt thereof, or a mixture of any of the foregoing, wherein:
R³ is 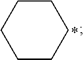
wherein * indicates the point of attachment to R².

8. The compound according to claim 1, which is selected from the group consisting of:
3-{[(4-methylpiperazin-1-yl)acetyl]amino}-N-[5-(piperidin-1-yl)-1,3,4-thiadiazol-2-yl]-4-(trifluoromethoxy)benzamide,
tert-butyl 4-(5-{[3-{[(4-methylpiperazin-1-yl)acetyl]amino}-4-(trifluoromethoxy)benzoyl]amino}-1,3,4-thiadiazol-2-yl)piperazine-1-carboxylate,
tert-butyl 4-[5-({3-[(morpholin-4-ylacetyl)amino]-4-(trifluoromethoxy)benzoyl}amino)-1,3,4-thiadiazol-2-yl]piperazine-1-carboxylate, p1 3-{[(4-methylpiperazin-1-yl)acetyl]amino}-N-[5-(pyrrolidin-1-yl)-1,3,4-thiadiazol-2-yl]-4-(trifluoromethoxy)benzamide,
N-(5-cyclohexyl-1,3,4-thiadiazol-2-yl)-3-{[(4-methylpiperazin-1-yl)acetyl]amino}-4-(trifluoromethoxy)benzamide,
methyl 4-(5-{[3-{[(4-methylpiperazin-1-yl)acetyl]amino}-4-(trifluoromethoxy)benzoyl]amino}-1,3,4-thiadiazol-2-yl)piperazine-1-carboxylate,
3-{[(4-methylpiperazin-1-yl)acetyl]amino}-N-[5-(4-methylpiperidin-1-yl)-1,3,4-thiadiazol-2-yl]-4-(trifluoromethoxy)benzamide,
N-[5-(4-hydroxy-4-methylpiperidin-1-yl)-1,3,4-thiadiazol-2-yl]-3-{[(4-methylpiperazin-1-yl)acetyl]amino}-4-(trifluoromethoxy)benzamide,
N-{5-[4-(cyclopropylcarbonyl)piperazin-1-yl]-1,3,4-thiadiazol-2-yl}-3-{[(4-methylpiperazin-1-yl)acetyl]amino}-4-(trifluoromethoxy)benzamide,
N-{5-[4-(2-hydroxypropan-2-yl)piperidin-1-yl]-1,3,4-thiadiazol-2-yl}-3-[(morpholin-4-ylacetyl)amino]-4-(trifluoromethoxy)benzamide,
N-[5-(4-methoxypiperidin-1-yl)-1,3,4-thiadiazol-2-yl]-3-{[(4-methylpiperazin-1-yl)acetyl]amino}-4-(trifluoromethoxy)benzamide,
4-(2-{[5-(4,4-dimethylpiperidin-1-yl)-1,3,4-thiadiazol-2-yl]carbamoyl}-2-(trifluoromethoxy)phenyl]amino}-2-oxoethyl)-1-methylpiperazin-1ium hexafluorophosphate,
ethyl 4-[5-({3-[(morpholin-4-ylacetyl)amino]-4-(trifluoromethoxy)benzoyl}amino)-1,3,4-thiadiazol-2-yl]piperazine-1-carboxylate,
N-[5-(4-hydroxypiperidin-1-yl)-1,3,4-thiadiazol-2-yl]-3-{[(4-methylpiperazin-1-yl)acetyl]amino}-4-(trifluoromethoxy)benzamide,
N-{5-[4-(dimethylsulfamoyl)piperazin-1-yl]-1,3,4-thiadiazol-2-yl}-3-[(morpholin-4-ylacetyl)amino]-4-(trifluoromethoxy)benzamide,
N,N-dimethyl-4-(5-{[3-{[(4-methylpiperazin-1-yl)acetyl]amino}-4-(trifluoromethoxy)benzoyl]amino}-1,3,4-thiadiazol-2-yl)piperazine-1-carboxamide,
ethyl 4-(5-{[3-{[(4-methylpiperazin-1-yl)acetyl]amino}-4-(trifluoromethoxy)benzoyl]amino }-1,3,4-thiadiazol-2-yl)piperazine-1-carboxylate,
N-[5-(4-acetylpiperazin-1-yl)-1,3,4-thiadiazol-2-yl]-3-{[(4-methylpiperazin-1-yl)acetyl]amino }-4-(trifluoromethoxy)benzamide,
N-{5-[4-(dimethylsulfamoyl)piperazin-1-yl]-1,3,4-thiadiazol-2-yl}-3-{[(4-methylpiperazin-1-yl)acetyl]amino}-4-(trifluoromethoxy)benzamide,
N-{5-[4-(cyclopropylcarbonyl)piperazin-1-yl]-1,3,4-thiadiazol-2-yl}-3 -[(morpholin-4-ylacetyl)amino]-4-(trifluoromethoxy)benzamide,
methyl 1-[5({3-[(morpholin-4-ylacetyl)amino]-4-(trifluoromethoxy)benzoyl}amino)-1,3,4-thiadiazol-2-yl]piperidine-4-carboxylate,
methyl 1-(5-{[3-{[(4-methylpiperazin-1-yl)acetyl]amino}-4-(trifluoromethoxy)benzoyl]amino}-1,3,4-thiadiazol-2-yl)piperidine-4-carboxylate,
N-(5-cyclohexyl-1,3,4-thiadiazol-2-yl)-3-[(morpholin-4-ylacetyl)amino]-4-(trifluoromethoxy)benzamide, and
methyl 4-[5-({3-[(morpholin-4-ylacetyl)amino]-4-(trifluoromethoxy)benzoyl }amino)-1,3,4-thiadiazol-2-yl]piperazine-1-carboxylate,
or a tautomer, an N-oxide, a hydrate, a solvate, or a salt thereof, or a mixture of any of the foregoing.

9. (canceled)

10. A pharmaceutical composition comprising a compound of formula (I), or a stereoisomer, a tautomer, an N oxide, a hydrate, a solvate, a salt, or a pharmaceutically acceptable salt thereof, or a mixture of any of the foregoing, according to claim 1, and a pharmaceutically acceptable diluent or carrier.

11. A pharmaceutical combination comprising:
one or more first active ingredients selected from a compound of formula (I), or a tautomer, an N-oxide, a hydrate, a solvate, or a salt thereof, or a mixture of any of the foregoing, according to claim 1, and
one or more second active ingredients selected from chemotherapeutic anti cancer agents.

12-13. (canceled)

14. A method for prophylaxis or treatment of a disease comprising administering to a patient in need thereof a therapeutically effective amount of the compound of formula (I) according to claim 1, or a tautomer, an N-oxide, a hydrate, a solvate, or a salt thereof, or a mixture of any of the foregoing, wherein said disease is a disease in which aberrant Wnt signalling is implicated in a patient.

15. The method according to claim 14, wherein the disease is a genetic disease caused by mutations in Wnt signaling components.

16. The method according to claim 14, wherein the disease is a disease of uncontrolled cell growth, proliferation and/or survival, an inappropriate cellular immune response, or an inappropriate cellular inflammatory response.

17. The method of claim 16, wherein the disease of uncontrolled cell growth, proliferation and/or survival, an inappropriate cellular immune response, or an inappropriate cellular inflammatory response is mediated by the Wnt pathway.

18. The method according to claim 17, wherein the disease of uncontrolled cell growth, proliferation and/or survival, inappropriate cellular immune response, or inappropriate cellular inflammatory response is a haematological tumour, a solid tumour and/or metastases thereof.

19. The method according to claim 18, wherein the haematological tumour, a solid tumour and/or metastases thereof is selected from the group consisting of leukaemias and myelodysplastic syndrome, malignant lymphomas, head and neck tumours including brain tumours and brain metastases, tumours of the thorax including non small cell and small cell lung tumours, gastrointestinal tumours, endocrine tumours, mammary and other gynaecological tumours, urological tumours including renal, bladder and prostate tumours, skin tumours, and sarcomas, and/or metastases thereof.

20. The method according to claim 15, wherein the genetic disease is selected from the group consisting of: polyposis coli, osteoporosispseudoglioma syndrome, familial exudative vitreoretinopathy, retinal angiogenesis, early coronary disease, tetra-amelia syndrome, Müllerian-duct regression and virilization, SERKAL syndrome, diabetes mellitus type 2, Fuhrmann syndrome, Al-Awadi/Raas-Rothschild/Schinzel phocomelia syndrome, odonto-onycho-dermal dysplasia, obesity, splithand/foot malformation, caudal duplication syndrome, tooth agenesis, Wilms tumor, skeletal dysplasia, focal dermal hypoplasia, autosomal recessive anonychia, neural tube defects, alpha-thalassemia (ATRX) syndrome, fragile X syndrome, ICF syndrome, Angelman syndrome, Prader-Willi syndrome, Beckwith-Wiedemarm Syndrome and Rett syndrome.
